(19) United States
(12) Patent Application Publication
(10) Pub. No.: US 2026/0305973 A1
(43) Pub. Date: Oct. 8, 2026

(54) INTRAORAL SENSORS FOR HEALTH MONITORING

(71) Applicant: Align Technology, Inc., San Jose, CA (US)

(72) Inventors: Yixin Xu, San Jose, CA (US); Sheyda Nazarian, San Jose, CA (US); Zijie Zhu, San Jose, CA (US); Byong-Ho Park, San Jose, CA (US); Vincent Yeh, Fremont, CA (US); Sangshik Park, San Jose, CA (US); Mitchell Bond, San Jose, CA (US)

(21) Appl. No.: 19/636,673

(22) Filed: Apr. 1, 2026

Related U.S. Application Data
(60) Provisional application No. 63/782,372, filed on Apr. 2, 2025.

(51) Int. Cl. A61B 5/08; A61B 5/00; A61B 90/00

(52) U.S. Cl. CPC A61B 5/0803; A61B 5/0816; A61B 5/4809; A61B 5/4815; A61B 5/4818; A61B 5/682; A61B 5/7264; A61B 5/742; A61B 90/06; A61B 2090/064; A61B 2562/16

(57) ABSTRACT

Systems and devices for monitoring a subject's respiration are provided. In some embodiments, a device includes a polymeric shell including a plurality of tooth-receiving cavities, where the polymeric shell is configured to be worn on the subject's teeth. The device can also include a force sensor coupled to the polymeric shell. The force sensor can be configured to measure force data associated with a deformation of the polymeric shell, where the deformation corresponds to respiration of the subject. The device can also include one or more processors and a memory having instructions that, when executed by the one or more processors, cause the one or more processors to perform operations including receiving the force data, and determining a respiratory parameter of the respiration based on the force data.

### CROSS-REFERENCE TO RELATED APPLICATION(S)

The present application claims the benefit of priority to U.S. Provisional Application No. 63/782,372, filed Apr. 2, 2025, the disclosure of which is incorporated by reference herein in its entirety.

### TECHNICAL FIELD

The present technology generally relates to medical devices, and in particular, to intraoral monitoring systems and devices.

### BACKGROUND

Many physiological processes relevant to health, such as respiration and blood circulation, occur in and/or around the oral cavity. Thus, the oral cavity provides a unique vantage point for monitoring such processes to infer an individual's current health status. However, the oral cavity is a humid and warm environment, which may present challenges in ensuring sensor functionality over time. Moreover, the dynamic nature of the oral cavity (e.g., due to movements of the jaws and tongue during sleeping, speaking, eating, etc.) may interfere with measurement accuracy and consistency. Accordingly, there remains a need for improved systems and methods for intraoral monitoring.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a partially schematic illustration providing a general overview of an intraoral monitoring system, in accordance with embodiments of the present technology.

FIGS. 2A-2E illustrate a representative example of an intraoral monitoring device, in accordance with embodiments of the present technology.

FIG. 3A illustrates another representative example of an intraoral monitoring device, in accordance with embodiments of the present technology.

FIG. 3B illustrates another representative example of an intraoral monitoring device, in accordance with embodiments of the present technology.

FIG. 3C illustrates another representative example of an intraoral monitoring device, in accordance with embodiments of the present technology.

FIGS. 4A and 4B are schematic illustrations of an intraoral monitoring device for monitoring a subject's respiration based on temperature-induced deformation, in accordance with embodiments of the present technology.

FIGS. 5A and 5B are graphs illustrating force data (FIG. 5A) and temperature data (FIG. 5B) measured by an intraoral monitoring device during mouth breathing and nasal breathing, in accordance with embodiments of the present technology.

FIGS. 6A and 6B are graphs illustrating force data as measured by intraoral monitoring devices fabricated using additive manufacturing (FIG. 6A) and thermoforming (FIG. 6B), in accordance with embodiments of the present technology.

FIGS. 7A and 7B are graphs illustrating force and temperature data measured by intraoral monitoring devices having a thermosensitive material (FIG. 7A) and a non-thermosensitive material (FIG. 7B), in accordance with embodiments of the present technology.

FIG. 8A is a side cross-sectional view of a pressure sensor, in accordance with embodiments of the present technology.

FIG. 8B is a side cross-sectional view of a pressure sensor including a gel, in accordance with embodiments of the present technology.

FIG. 8C is a side cross-sectional view of a pressure sensor including a gel and a biocompatible coating, in accordance with embodiments of the present technology.

FIGS. 8D and 8E illustrate a process for encapsulating a pressure sensor, in accordance with embodiments of the present technology.

FIG. 9 is a graph illustrating validation results for the encapsulated pressure sensor of FIGS. 8D and 8E, in accordance with embodiments of the present technology.

FIG. 10 is a graph illustrating acceleration data as measured by an inertial measurement unit (IMU) of an intraoral monitoring device, in accordance with embodiments of the present technology.

FIG. 11 is a graph illustrating force data measured by a force sensor of an intraoral monitoring device during simulated bruxism events, in accordance with embodiments of the present technology.

FIGS. 12A and 12B are graphs illustrating acceleration data (FIG. 12A) and angular velocity data (FIG. 12B) measured by an IMU of an intraoral monitoring device during simulated bruxism events, in accordance with embodiments of the present technology.

FIG. 13 illustrates monitoring of a subject's cardiac activity via head ballistocardiography (BCG), in accordance with embodiments of the present technology.

FIG. 14 is a graph illustrating head BCG data measured by a motion sensor of an intraoral monitoring device, in accordance with embodiments of the present technology.

FIGS. 15A and 15B are graphs illustrating heart rate data estimated from head BCG determined by an intraoral monitoring device during rest (FIG. 15A) and after exercise (FIG. 15B), in accordance with embodiments of the present technology.

FIG. 16 is a flow diagram illustrating a method for intraoral monitoring, in accordance with embodiments of the present technology.

FIG. 17 illustrates an example intraoral monitoring device including a monitoring unit encapsulated using double-layer thermoforming, in accordance with embodiments of the present technology.

FIGS. 18A-18C illustrate an example encapsulation process for forming an intraoral monitoring device, in accordance with embodiments of the present technology.

FIGS. 19A and 19B are graphs illustrating example pressure sensitivity measurements for an encapsulated pressure sensor, in accordance with embodiments of the present technology.

FIGS. 20A-20D are graphs illustrating example pressure and force data obtained using encapsulated pressure and force sensors, in accordance with embodiments of the present technology.

FIG. 21 shows graphs illustrating pressure data (top) and temperature data (bottom) obtained using encapsulated pressure and temperature sensors, in accordance with embodiments of the present technology.

FIGS. 22A and 22B illustrate a process for forming an intraoral monitoring device with a preformed pocket via thermoforming, in accordance with embodiments of the present technology.

FIGS. 22C and 22D illustrate a process for forming an intraoral monitoring device with a preformed pocket via direct fabrication, in accordance with embodiments of the present technology.

FIGS. 23A and 23B illustrate a thermoformed intraoral monitoring device with a transparent epoxy (FIG. 23A) and a colored epoxy (FIG. 23B), in accordance with embodiments of the present technology.

FIGS. 23C and 23D illustrate a directly fabricated intraoral monitoring device with a transparent epoxy (FIG. 23C) and a colored epoxy (FIG. 23D), in accordance with embodiments of the present technology.

FIGS. 24A-24D are graphs illustrating example intraoral test results for an intraoral monitoring device formed using direct encapsulation processes, in accordance with embodiments of the present technology.

FIGS. 25A-25D illustrate an intraoral monitoring device with a pre-encapsulated monitoring unit, in accordance with embodiments of the present technology.

FIG. 26 illustrates an intraoral monitoring device having an encapsulated monitoring unit with a stacked configuration, in accordance with embodiments of the present technology.

FIGS. 27A and 27B illustrate top and bottom views, respectively, of an example monitoring unit of an intraoral monitoring device having a charging coil, in accordance with embodiments of the present technology.

FIGS. 28A and 28B illustrate top and bottom views, respectively, of an intraoral monitoring device including a preformed pocket for housing a monitoring unit, in accordance with embodiments of the present technology.

FIGS. 29A and 29B illustrate a charging device including an integrated teeth mold for aligning and charging an intraoral monitoring device, in accordance with embodiments of the present technology.

FIGS. 30A and 30B illustrate a charging device with a slide-in design for aligning and charging an intraoral monitoring device, in accordance with embodiments of the present technology.

FIGS. 31A and 31B illustrate another charging device with a slide-in design for aligning and charging an intraoral monitoring device, in accordance with embodiments of the present technology.

FIGS. 32A and 32B illustrate a charging device with a clipping design for aligning and charging an intraoral monitoring device, in accordance with embodiments of the present technology.

FIG. 33 illustrates a charging device with a hook design for aligning and charging an intraoral monitoring device, in accordance with embodiments of the present technology.

FIGS. 34A and 34B illustrate another charging device with a hook design for aligning and charging an intraoral monitoring device, in accordance with embodiments of the present technology.

FIG. 35 illustrates a charging device with an alignment-free design for charging an intraoral monitoring device, in accordance with embodiments of the present technology.

FIGS. 36A and 36B illustrate another charging device with an alignment-free design for charging an intraoral monitoring device, in accordance with embodiments of the present technology.

FIGS. 37A and 37B illustrate another charging device with a clipping design for aligning and charging an intraoral monitoring device, in accordance with embodiments of the present technology.

FIG. 38A illustrates a representative example of a tooth repositioning appliance configured in accordance with embodiments of the present technology.

FIG. 38B illustrates a tooth repositioning system including a plurality of appliances, in accordance with embodiments of the present technology.

FIG. 38C illustrates a method of orthodontic treatment using a plurality of appliances, in accordance with embodiments of the present technology.

FIG. 39 illustrates a method for designing an orthodontic appliance, in accordance with embodiments of the present technology.

FIG. 40 illustrates a method for digitally planning an orthodontic treatment and/or design or fabrication of an appliance, in accordance with embodiments of the present technology.

FIG. 41 is a partially schematic diagram providing a general overview of an additive manufacturing process, in accordance with embodiments of the present technology.

### DETAILED DESCRIPTION

The present technology relates to systems, devices, and methods for monitoring physiological parameters of a subject from within the subject's oral cavity. In some embodiments, for example, a system includes an intraoral monitoring device configured to be positioned within a subject's mouth (e.g., a dental appliance, such as an aligner, a retainer, a palatal expander, etc.). The intraoral monitoring device can include one or more sensors (e.g., a force sensor, a motion sensor, a temperature sensor, and/or a pressure sensor). The one or more sensors can be configured to detect physiological changes in and/or around the oral cavity, and these physiological changes may be indicative of characteristics of physiological parameters associated with respiration, cardiac activity, sleep, bruxism, and more.

In some embodiments, for example, a device for monitoring a subject's respiration includes a polymeric shell having a plurality of teeth-receiving cavities, where the polymeric shell is configured to be worn on the subject's teeth. The device can further include a force sensor coupled to the polymeric shell, where the force sensor is configured to measure force data associated with a deformation of the polymeric shell. In some embodiments, the deformation corresponds to respiration (e.g., inhalation and/or exhalation) of the subject. For instance, respiration of the subject can result in thermal expansion and/or contraction of the polymeric shell, resulting in deformations over time that may be measured by the force sensor. The device can further include one or more processors, and a memory having instructions that, when executed by the one or more processors, cause the one or more processors to perform operations including receiving the force data, and determining a respiratory parameter of the respiration based on the force data. For instance, it may be determined from the force data whether the subject is primarily breathing through their mouth (“mouth breathing”) as opposed to breathing through their nose (“nasal breathing”) during sleep.

As another example, in some embodiments, a device for monitoring a subject's cardiac activity includes a polymeric shell having a plurality of teeth-receiving cavities, where the polymeric shell is configured to be worn on the subject's teeth. The device can further include a motion sensor coupled to the polymeric shell, the motion sensor configured to measure motion data associated with blood flow in the subject's head (e.g., head ballistocardiogram data). The device can further include one or more processors, and a memory having instructions that, when executed by the one or more processors, cause the one or more processors to perform operations including receiving the motion data, and determining the cardiac parameter based on the motion data. For instance, a heart rate of the subject may be determined based on the motion data.

The present technology can provide numerous advantages compared to conventional approaches for monitoring physiological processes. For instance, the intraoral monitoring devices described herein can be used to measure many different types of physiological parameters in a minimally invasive manner from a relatively stable position within the intraoral cavity, whereas other types of monitoring devices (e.g., wearable devices such as fitness watches) may experience significant perturbations from external sources. This may be particularly advantageous during sleep, where the oral cavity and head may maintain a relatively stable position whereas the rest of the body may not. Further, the oral cavity may provide access to physiological parameters that are difficult or impossible to measure accurately using monitoring devices located elsewhere in the body (e.g., respiration, bruxism). Moreover, the intraoral monitoring devices may serve dual monitoring and treatment purposes, e.g., an intraoral monitoring device may be able to both determine intraoral physiological parameters (e.g., respiration rate) and treat the subject's teeth (e.g., reposition the teeth according to an orthodontic treatment plan, maintain the teeth in a desired position after orthodontic treatment).

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” “lower,” “left,” “right,” etc., can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

The headings provided herein are for convenience only and do not interpret the scope or meaning of the claimed present technology. Embodiments under any one heading may be used in conjunction with embodiments under any other heading. For example, any of the embodiments described in Sections I.A-I.E below may be combined with each other, any of the embodiments described in Sections I-V below may be combined with each other, etc.

### I. Intraoral Monitoring Devices

The present technology relates to systems and devices for intraoral monitoring. In some embodiments, an intraoral monitoring system includes an intraoral monitoring device configured to be worn on a subject's teeth. For instance, the intraoral monitoring device may be a dental appliance, such as an aligner, a retainer, a palatal expander, etc., that includes a shell having a plurality of teeth-receiving cavities for receiving the subject's teeth. The intraoral monitoring device may further include one or more sensors coupled to the shell. The sensors may be configured to generate sensor data (e.g., force data, motion data, temperature data, pressure data) from within the subject's oral cavity. The sensor data may be analyzed to determine one or more physiological parameters of the subject (e.g., respiratory parameters, cardiac parameters), which in turn may be analyzed to determine a subject's disease, condition, and/or state, such as the subject's sleep quality, current activity level, cardiac health, risk of a cardiac event, etc. In some embodiments, the intraoral monitoring device is configured to be worn substantially continuously by the subject (e.g., the subject may temporarily remove the device when eating, drinking, or brushing their teeth but otherwise wears the device at all times). Alternatively, the intraoral monitoring device may be worn by the subject only for prescribed periods of time, such as while the subject is sleeping, engaging in physical activity, etc.

FIG. 1 is a partially schematic illustration providing a general overview of an intraoral monitoring system 100, in accordance with embodiments of the present technology. In some embodiments, the system 100 can be used to determine one or more physiological parameters related to a subject's health, condition, behavior, and/or activity. For example, the system 100 can be used to monitor the subject's sleep parameters (e.g., sleep duration, sleep depth, sleep consistency, sleep quality, sleep stages, apnea events, snoring, sleep bruxism), cardiac parameters (e.g., cardiac output, heart rate, heart rate variability), respiratory parameters (e.g., respiration rate, respiration rhythm), etc.

The system 100 can include an intraoral monitoring device 102 (“intraoral device 102”) and optionally, an external device 104. In some embodiments, the intraoral device 102 is a dental appliance configured to be worn in the subject's mouth. For instance, the intraoral device 102 can be an aligner, retainer, palatal expander, mouthguard, nightguard, oral sleep apnea device, splint, etc. Additional examples and details of dental appliances that may be used for the intraoral device 102 are provided in Section III below. In the illustrated embodiment, the intraoral device 102 includes a shell 106 having a plurality of teeth-receiving cavities for receiving some or all of the teeth of a dental arch of the subject (e.g., an upper dental arch or a lower dental arch). The shell 106 can be a polymeric shell that is fabricated via thermoforming and/or additive manufacturing, as will be described further in Section II below.

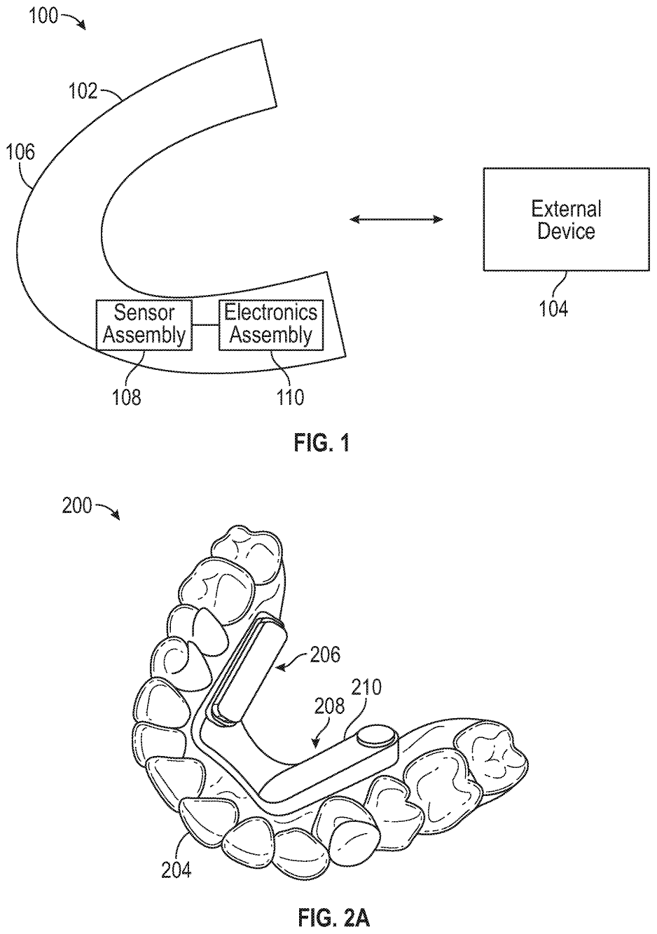

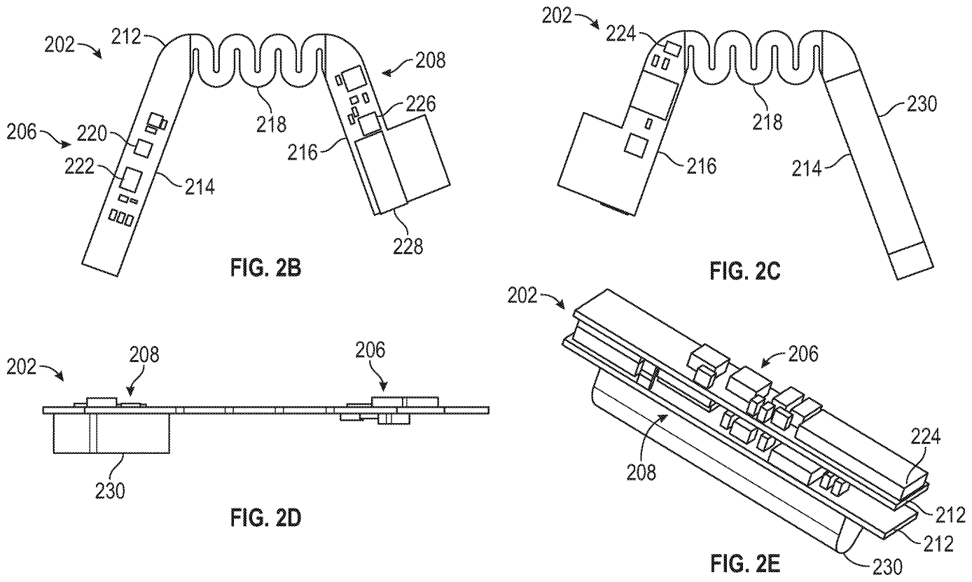

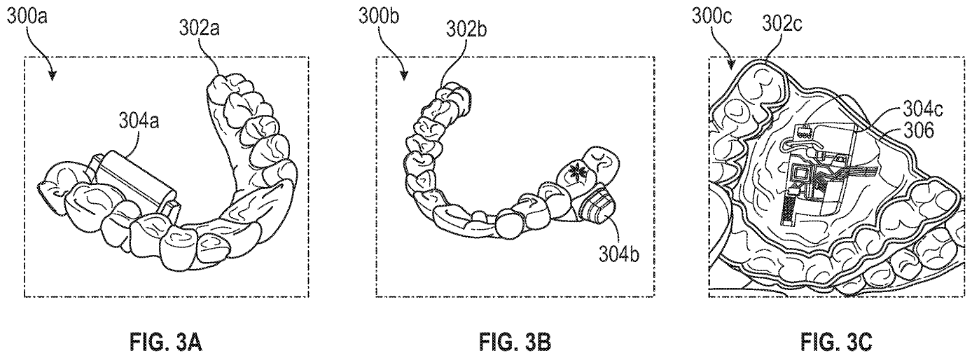

In some embodiments, the shell 106 is coupled to a monitoring unit that includes a sensor assembly 108 and an electronics assembly 110. For instance, the shell 106 can optionally include a preformed pocket (or other cavity, receptacle, recess, etc., formed in the shell 106), and the sensor assembly 108 and the electronics assembly 110 can be disposed in the preformed pocket. However, the sensor assembly 108 and the electronics assembly 110 may be disposed in a variety of other locations with respect to the shell 106, e.g., these components may each independently be coupled to an external surface of the shell 106 (a surface facing away from the received teeth), coupled to an internal surface of the shell 106 (a surface facing toward the received teeth), embedded within a sidewall of the shell 106, etc. The sensor assembly 108 and/or electronic assembly 110 may each independently be located at a buccal portion of the shell 106, a lingual portion of the shell 106, an occlusal portion of the shell 106, a gingival portion of the shell 106, an anterior portion of the shell 106, a posterior portion of the shell 106, a palatal portion of the shell 106, or any other suitable location. Example configurations that may be used for the shell 106, sensor assembly 108, and electronics assembly 110 are discussed further below, e.g., in connection with FIGS. 2A-3C.

The sensor assembly 108 can include one or more sensors configured to generate sensor data indicative of physiological parameters corresponding to physiological processes that occur within and/or proximate to the oral cavity. For example, the sensors can be configured to measure force, pressure, temperature, motion, humidity, air flow, etc., which may be correlated to physiological parameters such as respiration rate and heart rate. In some embodiments, the sensor assembly 108 includes one or more of the following: a force sensor (e.g., a load cell, strain gauge, piezoelectric sensor), a pressure sensor (e.g., a microelectromechanical systems (MEMS)-based sensor, strain gauge, piezoelectric sensor, capacitive sensor), a temperature sensor (e.g., a thermistor, a thermocouple), a motion sensor (e.g., an accelerometer, a gyroscope, an inertial measurement unit (IMU)), a humidity sensor, a proximity sensor, an optical sensor, a contact sensor, or any other suitable sensor. For instance, the sensor assembly 108 may include a force sensor configured to measure force data associated with a deformation of the shell, where the force data may be correlated to respiration of the subject as discussed in Section I.A below. Alternatively or in combination, the sensor assembly 108 may include a motion sensor configured to measure motion data associated with movements of the subject's head, where the motion data may be correlated to cardiac activity of the subject as discussed in Section I.B below.

The electronics assembly 110 can be operably coupled to the sensor assembly 108 and can include electronic components configured to support the operations of the sensor assembly 108. For instance, the electronics assembly 110 can include communication circuitry, processing circuitry, memory, and/or power management circuitry. In some embodiments, the electronics assembly 110 is configured to store, process, and/or analyze the sensor data produced by the sensor assembly 108. Further, the electronics assembly 110 can control the sensor assembly 108, e.g., the electronics assembly 110 may direct one or more sensors of the sensor assembly 108 to begin measuring, to cease measuring, etc.

Moreover, the electronics assembly 110 may be configured to power the sensor assembly 108. For instance, the electronics assembly 110 can include a power source configured to supply power to the sensor assembly 108 and/or the rest of the intraoral device 102. Further, in some embodiments, the electronics assembly 110 includes charging circuitry that is configured to be in electrical communication with an external charging device for recharging the power source. The charging circuitry may be configured to wirelessly receive power from the external charging device. For instance, the charging circuitry may include a first coil, and the external charging device may include a second coil that can be aligned with the first coil to transfer power thereto. In some embodiments, this alignment is at least partially facilitated by a coupling mechanism, e.g., as described below in Section III.

In some embodiments, the electronics assembly 110 is configured to communicate with the external device 104. For instance, the electronics assembly 110 can be configured to transmit data (e.g., sensor data) received from the sensor assembly 108 to the external device 104. As will be described further herein, the external device 104 may be configured to receive and process the sensor data to determine physiological parameters for the subject. Alternatively or in combination, the electronics assembly 110 can be configured to receive data (e.g., control instructions) from the external device 104, and to transmit the received data to the sensor assembly 108.

The external device 104 can be any computing device, such as a mobile device (e.g., smartphone, smartwatch, tablet), laptop, personal computer, workstation, remote server, or smart appliance case. The computing device may be part of a virtual dental care system as described in, e.g., U.S. Patent Application Publication No. 2022/0023003, the disclosure of which is incorporated by reference herein in its entirety. In some embodiments, the external device 104 is configured to communicate information from the intraoral device 102 to a user (e.g., the subject, a clinician). Alternatively or in combination, the external device 104 may be configured to communicate information from the user to the intraoral device 102 (e.g., instructions for controlling one or more operations of the intraoral device 102). Optionally, the external device 104 may be configured to communicate information from the intraoral device 102 and/or from the user to a remote server or other computing device.

While the external device 104 is shown in FIG. 1, in some embodiments, the external device 104 is omitted from the intraoral monitoring system 100. For instance, processing, transmitting, and/or storing of data may occur directly in the intraoral device 102, such as via the electronics assembly 110.

FIGS. 2A-2E illustrate a representative example of an intraoral monitoring device 200, in accordance with embodiments of the present technology. Specifically, FIG. 2A is a perspective view of the device 200, FIG. 2B is a top view of a monitoring unit 202 of the device 200, FIG. 2C is a bottom view of the monitoring unit 202, FIG. 2D is a side view of the monitoring unit 202, and FIG. 2E is a close-up view of a stacked configuration that may be used for the monitoring unit 202.

Referring to FIGS. 2A-2E collectively, the device 200 is an example of the intraoral device 102 of the intraoral monitoring system 100 of FIG. 1, and can be the same or generally similar to the intraoral device 102. For instance, the device 200 can include a shell 204, and a monitoring unit 202 coupled to the shell, where the monitoring unit 202 includes a sensor assembly 206 and an electronics assembly 208. Further, while not depicted in FIGS. 2A-2E, the device 200 can be configured to communicate with an external device, such as the external device 104 of the system 100 of FIG. 1.

Referring now to FIG. 2A, the shell 204 can include a preformed pocket 210, and the sensor assembly 206 and the electronics assembly 208 (obscured in FIG. 2A) can be disposed in the preformed pocket 210. The preformed pocket 210 may be located at a lingual portion of the shell 204. As shown, the preformed pocket 210 can be U-shaped, e.g., to conform to the shape of the lingual portion of the shell 204. Alternatively, however, the preformed pocket 210 can be any suitable geometry, such as linear, planar, L-shaped, spherical, ellipsoidal, cylindrical, prismatic, etc. Moreover, the preformed pocket 210 can be located at a different portion of the shell 204, such as a buccal or occlusal portion of the shell 204. In some embodiments, the preformed pocket 210 is positioned on only one side of the shell 204 (e.g., only on a left side, a right side, or an anterior side of the shell 204). The preformed pocket 210 can be fabricated in many ways, as will be described further in Section II.

In some embodiments, the preformed pocket 210 at least partially or fully encloses a monitoring unit 202. Referring now to FIGS. 2B-2D together, the monitoring unit 202 can include a substrate 212, and the sensor assembly 206 and the electronics assembly 208 can be disposed on the substrate 212. In some embodiments, the substrate 212 is a printed circuit board (PCB), a flexible circuit board, or a combination thereof. The substrate 212 may include circuit traces that electrically connect one or more components of the sensor assembly 206 and/or the electronics assembly 208 to each other. The substrate 212 can conform to the shape of the shell 204 and/or the preformed pocket 210, e.g., the substrate 212 may be substantially U-shaped, similar to the preformed pocket 210. For instance, as shown, the substrate 212 may include a first end segment 214, a second end segment 216, and a flexible segment 218 positioned between the first end segment 214 and the second end segment 216. The flexible segment 218 may be configured to bend and/or twist to accommodate deformation of the shell 204, e.g., during placement of the shell 204 on the subject's teeth and/or removal of the shell 204 from the subject's teeth.

In some embodiments, the sensor assembly 206 and the electronics assembly 208 are positioned on opposite sides of the substrate 212, e.g., the sensor assembly 206 is located on the first end segment 214 and the electronics assembly 208 is located on the second end segment 216. In other embodiments, the sensor assembly 206 and electronics assembly 208 can be located at the same portion of the substrate (e.g., both assemblies 206, 208 are on the first end segment 214 or the second end segment 216), or individual components of the sensor assembly 206 and the electronics assembly 208 may have an intermixed arrangement. For instance, some components of the sensor assembly 206 and the electronics assembly 208 can be located on the first end segment 214 while other components of the sensor assembly 206 and the electronics assembly 208 can be located on the second end segment 216.

The sensor assembly 206 may include any of the features of the sensor assembly 108 of FIG. 1. For example, the sensor assembly 206 can include one or more sensors configured to measure physiological parameters within and/or proximate to the oral cavity. In some embodiments, the sensor assembly 206 includes a force sensor 220 (FIG. 2B). The force sensor can be configured to measure force data associated with a deformation of the shell 204. The deformation of the shell can be caused by many physiological processes, such as respiration, bruxism, speaking, snoring, swallowing, etc. The force sensor 220 may be positioned at any suitable location with respect to the shell 204. For instance, the force sensor 220 can be positioned proximate to an occlusal surface of the shell 204, a lingual surface of the shell 204, a buccal surface of the shell 204, or a combination thereof. Moreover, the force sensor 220 may be encapsulated by a biocompatible and/or thermosensitive material, as will be described further herein. In some embodiments, the force sensor 220 abuts a surface of the shell 204, and the force sensor 220 is configured to detect forces applied directly onto the force sensor 220 by the surface of the shell 204. Alternatively or in combination, the force sensor 220 can be configured to measure forces applied to the substrate 212 by the shell 204.

Alternatively or in combination, the sensor assembly 206 can include a pressure sensor 222 (FIG. 2B). The pressure sensor 222 can be configured to measure pressure data associated with air flow dynamics within the oral cavity. Changes in pressure within the oral cavity can be caused by many physiological processes, such as respiration, bruxism, speaking, snoring, swallowing, etc. The pressure sensor 222 can be positioned at any suitable location with respect to the shell 204, such as any of the locations described above in connection with the force sensor 220. Moreover, the pressure sensor 222 may be encapsulated by a biocompatible and/or water-resistant material, as will be described further herein.

Alternatively or in combination, the sensor assembly 206 can include a temperature sensor 224 (FIG. 2C). The temperature sensor 224 can be configured to measure temperature data associated with temperature changes within the oral cavity. Changes in temperature within the oral cavity can be caused by many physiological processes, such as respiration, speaking, snoring, swallowing, etc. The temperature sensor 224 can be positioned at any suitable location with respect to the shell 204, such as any of the locations described above in connection with the force sensor 220.

Alternatively or in combination, the sensor assembly 206 can include a motion sensor 226 (FIG. 2B). The motion sensor 226 can be configured to measure motion data associated with movement within and/or around the oral cavity. Movement within and/or around the oral cavity can be caused by many physiological processes, such as respiration, bruxism, speaking, snoring, swallowing, cardiac activity, etc. The motion sensor 226 can be positioned anywhere with respect to the shell 204, such as any of the locations described above in connection with the force sensor 220.

In some embodiments, components of the sensor assembly 206 are positioned in locations that are configured to amplify, reduce, and/or isolate signals of interest. For instance, the force sensor 220 may be positioned at a location along the monitoring unit 202 that is expected to experience the largest amount of deformation, the temperature sensor 224 may be positioned at a location along the monitoring unit 202 that is expected to experience the most change in temperature, etc. Optionally, sensors may be positioned in locations that target certain physiological processes, e.g., changes in force, pressure, temperature, motion, etc., that are associated with certain physiological processes of interest (e.g., snoring, bruxism, sleep apnea, heart failure) may be detected with greater sensitivity and/or accuracy in certain locations of the oral cavity, and thus the appropriate sensors may be positioned at or proximate to those locations. Many variations in sensor position (e.g., lingual, buccal, palatal) and/or orientation (e.g., inward, outward, upward, downward) are possible, as previously discussed.

The electronics assembly 208 is operably coupled to the sensor assembly 206 and includes electronic components configured to support the operations of the sensor assembly 206. The electronics assembly 208 may include any of the features of the electronics assembly 110 of FIG. 1. For example, the electronics assembly 208 may include one or more of communication circuitry, processing circuitry, memory, and/or power management circuitry. For instance, the electronics assembly 208 can include a microprocessor 228 (FIG. 2B). The microprocessor 228 can be configured to receive sensor data from the sensor assembly 206, and determine one or more physiological parameters related to the subject's health, condition, behavior, and/or activity based on the sensor data.

Optionally, the electronics assembly 208 can include a power source 230 (FIGS. 2C and 2D) configured to supply energy to the device 200 (e.g., to the sensor assembly 206). In some embodiments, the power source 230 is positioned on a bottom surface of the substrate 212, as depicted in FIGS. 2C and 2D. The power source 230 can be a rechargeable power source including a coil for inductive charging, e.g., as will be described further in Section III.

Although FIGS. 2B-2D depict the monitoring unit as having a U-shaped configuration, other configurations may be used. For instance, as shown in FIG. 2E, the monitoring unit 202 can instead be arranged in a stacked configuration in which at least some components of the sensor assembly 206 and/or the electronics assembly 208 are vertically above or below one another. For instance, in some embodiments, the sensor assembly 206 and/or the electronics assembly 208 can be disposed on multiple substrates 212, and the substrates 212 may be stacked on one another. Optionally, the sensor assembly 206 can be disposed on a top surface of a first substrate 212, and the electronics assembly 208 can be disposed on a bottom surface of a second substrate 212 directly below the sensor assembly 206.

FIG. 3A illustrates another representative example of an intraoral monitoring device 300a, in accordance with embodiments of the present technology. The intraoral monitoring device 300a can be generally similar to the intraoral monitoring device 102 of FIG. 1 and the intraoral monitoring device 200 of FIGS. 2A-2E, except as described below. For instance, the intraoral monitoring device 300a can include a shell 302a and a preformed pocket 304a housing a monitoring unit including a sensor assembly and/or an electronics assembly. In some embodiments, the preformed pocket 304a is positioned on only one side of a lingual surface of the shell 302a (e.g., as opposed to spanning multiple lingual surfaces, as shown in FIG. 2A). Alternatively, the preformed pocket 304a may be positioned on a buccal and/or occlusal surface. The preformed pocket 304a can have a generally cylindrical shape. In some embodiments, the sensor assembly and/or the electronics assembly are stacked within the preformed pocket 304a, e.g., similar to the configuration shown in FIG. 2E.

FIG. 3B illustrates another representative example of an intraoral monitoring device 300b, in accordance with embodiments of the present technology. The intraoral monitoring device 300b can be generally similar to the intraoral monitoring device 102 of FIG. 1 and the intraoral monitoring device 200 of FIGS. 2A-2E, except as described below. For instance, the intraoral monitoring device 300b can include a shell 302b and a preformed pocket 304b housing a monitoring unit including a sensor assembly and/or an electronics assembly. In some embodiments, the preformed pocket 304b is positioned on a buccal surface of the shell 302b. Alternatively or in combination, the preformed pocket 304b may be positioned on a lingual and/or occlusal surface of the shell 302b. The preformed pocket 304b may have a compact, generally prismatic shape. In some embodiments, the sensor assembly and/or the electronics assembly are stacked within the preformed pocket 304b, e.g., similar to the configuration shown in FIG. 2E.

FIG. 3C illustrates another representative example of an intraoral monitoring device 300c, in accordance with embodiments of the present technology. The intraoral monitoring device 300c can be generally similar to the intraoral monitoring device 102 of FIG. 1 and the intraoral monitoring device 200 of FIGS. 2A-2E, except as described below. For instance, the intraoral monitoring device 300c can include a shell 302c and a preformed pocket 304c housing a monitoring unit including a sensor assembly and/or an electronics assembly. In some embodiments, the shell 302c includes a preformed pocket 304c housing a sensor assembly and/or an electronics assembly. In some embodiments, the preformed pocket 304c is positioned in a palatal portion 306 of the shell 302c. The preformed pocket 304c may have a generally planar and/or flattened shape. For instance, the sensor assembly and/or the electronics assembly may be arranged in a flattened configuration within the preformed pocket 304c.

Additional examples and details of configurations for intraoral monitoring devices that are applicable to the present technology are provided in U.S. Patent Application Publication No. 2024/0335149, the disclosure of which is incorporated by reference herein in its entirety.

### A. Respiration Monitoring

In some embodiments, the intraoral monitoring devices and systems described herein (e.g., the systems and devices described above in connection with FIGS. 1-3C) can be used to monitor a subject's respiration. Respiratory patterns and behaviors can be an important indicator of a subject's health, sleep quality, and/or activity levels. Examples of respiratory parameters that can be determined using the present technology include respiration rate, respiration rhythm, respiration depth, apnea events, hypopnea events, snoring, mouth breathing, nasal breathing, and more.

In some embodiments, the systems and devices herein may monitor a subject's respiration indirectly (e.g., without measuring air flow and/or pressure associated with respiration) rather than directly. For example, respiration may cause deformation of a portion of an intraoral monitoring device, such as a polymeric shell of the device. As the subject breathes, the shell can be cooled or warmed due to exposure to air of different temperatures (e.g., cooler air from the external environment versus warmer air from the lungs), thereby leading to deformation (e.g., thermal expansion and/or contraction) of the shell. This deformation may be measured and used to assess one or more respiratory parameters.

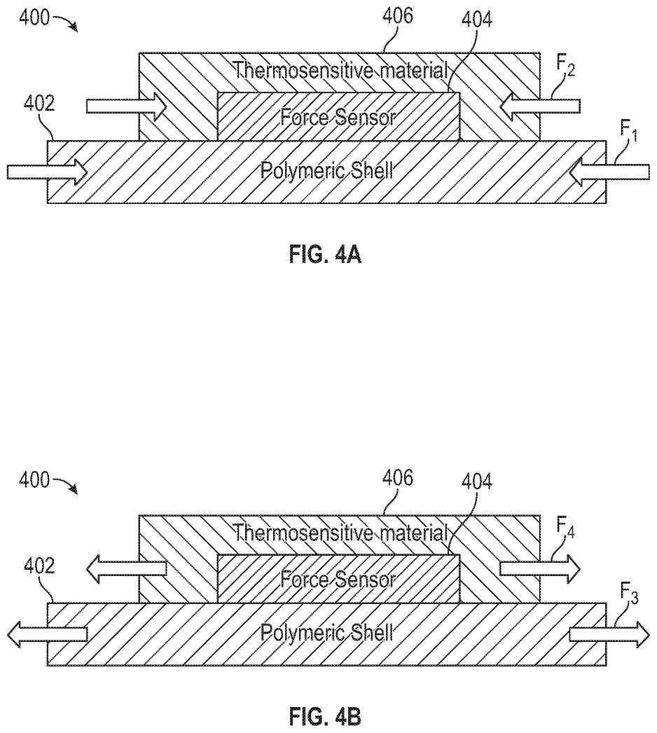

FIGS. 4A and 4B are schematic illustrations of an intraoral monitoring device 400 for monitoring a subject's respiration based on temperature-induced deformation, in accordance with embodiments of the present technology. Specifically, FIG. 4A illustrates a temperature-induced contraction of the device 400, and FIG. 4B illustrates a temperature-induced expansion of the device 400. Referring to FIGS. 4A and 4B together, the device 400 can be the same or generally similar to the systems and devices described in connection with FIGS. 1-3C. For instance, the device 400 can include a polymeric shell 402 and a sensor assembly including a force sensor 404 coupled to the polymeric shell 402. The force sensor 404 may optionally be encapsulated in a thermosensitive material 406 such as a thermosensitive polymer, e.g., polyurethane (e.g., thermoplastic polyurethane), polycarbonate, polyurethane, terephthalate glycol, ethylene vinyl acetate, polyethylene, polypropylene, polymethyl methacrylate, silicone rubber, polyurethane foams, nylon, thermoplastic elastomers, and/or a combination thereof. Further examples of thermosensitive materials include thermosensitive hydrogels, silicon-based materials, UV-curable potting materials (e.g., resin, Dymax, Loctite), epoxies (e.g., EP40TCND, EP40TCMED, EP40MED, EP40 White, MED-T7110, EP21AOLV-2MED, EP42HT-4AOMED), etc.

In some embodiments, the polymeric shell 402 and the thermosensitive material 406 (if present) contract and/or expand in response to temperature changes within the oral cavity. The polymeric shell 402 and/or the thermosensitive material 406 can be selected to have a coefficient of thermal expansion (CTE) that is sufficiently large to produce a measurable deformation over the range of temperatures within the oral cavity. For instance, the CTE of the polymeric shell 402 and/or the thermosensitive material 406 can each independently be at least 1.0 K−1×10−5, such as within a range from 1.0 K⁻¹×10⁻⁵ to 5.0 K⁻¹×10⁻⁵, 1.0 K⁻¹×10⁻⁵ to 7.0 K⁻¹×10⁻⁵, 1.0 K⁻¹×10⁻⁵ to 10 K⁻¹×10⁻⁵, 4 K⁻¹×10⁻⁵ to 10 K⁻¹×10⁻⁵, etc. In some embodiments, the CTE of the polymeric shell 402 can be 7.0 K⁻¹×10⁻⁵, and the CTE of the thermosensitive material 406 can be 4.0 K⁻¹×10⁻⁵.

Referring now to FIG. 4A, as the subject breathes in through their mouth (e.g., inhales), cool air from the ambient environment enters the oral cavity, thereby cooling the polymeric shell 402 and/or the thermosensitive material 406. In response to the cooling, the polymeric shell 402 and/or the thermosensitive material 406 can contract (e.g., decrease in length, width, height, surface area, volume, etc.). This contraction can cause the polymeric shell 402 to apply one or more inwardly directed forces F₁ on the force sensor 404, and/or cause the thermosensitive material 406 to apply one or more inwardly directed forces F₂ on the force sensor 404. The magnitude of the cooling can correlate to the magnitude of the inwardly directed forces F₁ and F₂, e.g., the larger the magnitude of the cooling, the larger the magnitude of the contraction and thus the larger the magnitude of the inwardly directed forces F₁ and F₂.

Referring next to FIG. 4B, as the subject breathes out through their mouth (e.g., exhales), warm air from within the subject's lungs exits the oral cavity, thereby warming the polymeric shell 402 and/or the thermosensitive material 406. In response to the warming, the polymeric shell 402 and/or the thermosensitive material 406 can expand (e.g., increase in length, width, height, surface area, volume, etc.). This expansion can cause the polymeric shell 402 to apply one or more outwardly directed forces F₃ on the force sensor 404, and/or cause the thermosensitive material 406 to apply one or more outwardly directed forces F₄ on the force sensor 404. The magnitude of the cooling can correlate to the magnitude of the outwardly directed forces F₃ and F₄, e.g., the larger the magnitude of the warming, the larger the magnitude of the expansion and thus the larger the magnitude of the outwardly directed forces F₃ and F₄.

In some embodiments, force data measured by the force sensor 404 may be correlated to one or more respiratory parameters. For instance, the forces applied to the force sensor 404 may change according to the changes in oral temperature, which correlate to the respiratory pattern of the subject as discussed above, such that the pattern (e.g., waveform) of the force data measured by the force sensor 404 corresponds to the subject's respiration pattern. Accordingly, the intraoral monitoring device 400 may use the force data to determine the subject's respiration rate, respiration depth, respiration rhythm, and/or other respiration characteristics. For instance, the period between two neighboring peaks in the force waveform may be used to determine respiration rate. As another example, the amplitude of the force waveform may be correlated to respiration depth (e.g., the volume of air that is inhaled and/or exhaled in a respiration cycle). In a further example, the overall pattern of the force waveform may be correlated to the respiration pattern. In yet another example, irregularities in the force waveform (e.g., periods where there are few or no changes in force) may be correlated to a decrease in or cessation of respiration (e.g., apnea or hypopnea events).

In some embodiments, the force data measured by the force sensor 404 can be used to determine whether a subject is mouth breathing or nasal breathing. For instance, during nasal breathing, temperature changes within the oral cavity may be relatively small (e.g., due to less direct exchange of air with the ambient environment), leading to smaller deformations of the shell 402 and/or the thermosensitive material 406. Thus, the magnitude of the forces applied to the force sensor 404 may be relatively small during nasal breathing. In contrast, during mouth breathing, temperature changes within the oral cavity are relatively large (e.g., the opening and closing of the mouth leads to direct exchange with the ambient environment), leading to larger deformations of the shell 402 and/or the thermosensitive material 406. Thus, the magnitude of forces applied to the force sensor 404 may be relatively large during mouth breathing.

In some embodiments, the intraoral monitoring device 400 may be configured to differentiate between mouth breathing and nasal breathing at least in part based on an amplitude of the force data, e.g., larger amplitudes may be correlated to mouth breathing and smaller amplitudes may be correlated to nasal breathing. In some embodiments, the subject is determined to be mouth breathing if the amplitude exceeds a force threshold value, and the subject is determined to be nasal breathing if the amplitude is below a force threshold value. The force threshold value may be customized to the subject (e.g., based on historical respiratory data of the subject) or may be a generic value that is used across multiple subjects (e.g., based on historical respiratory data for a larger population). For instance, the historical respiratory data of the subject and/or population may include first force data for a first period of time corresponding to mouth breathing and second force data for a second period of time corresponding to nasal breathing, where the first force data and the second force data may be used to determine a force threshold value to distinguish between mouth breathing and nasal breathing.

In some embodiments, the systems and devices herein can use a temperature sensor for monitoring a subject's respiration, additionally or alternatively to using a force sensor. For instance, a temperature sensor may be part of a sensor assembly of an intraoral monitoring device, and the temperature sensor can be configured to measure temperature data within the subject's oral cavity. The temperature data can correspond to the subject's respiration. For instance, changes in oral temperature may correlate to the respiratory pattern of the subject as discussed above, such that the pattern (e.g., waveform) of the temperature data measured by the temperature sensor corresponds to the subject's respiration pattern. Accordingly, the temperature data can be used to determine the subject's respiration rate, respiration depth, respiration rhythm, and/or other respiration characteristics. Further, the temperature data may be used to differentiate between mouth breathing and nasal breathing (e.g., higher oral temperatures may be correlated to periods of nasal breathing and lower oral temperatures may be correlated to periods of mouth breathing). For instance, the subject may be determined to be nasal breathing if an average temperature of the temperature data exceeds a temperature threshold value, and the subject may be determined to be mouth breathing if the average temperature is below a temperature threshold value. The temperature threshold value may be customized to the subject (e.g., based on historical respiratory data of the subject) or may be a generic value that is used across multiple subjects (e.g., based on historical respiratory data for a larger population). For instance, the historical respiratory data of the subject and/or population may include first temperature data for a first period of time corresponding to mouth breathing and second temperature data for a second period of time corresponding to nasal breathing, where the first temperature data and the second temperature data may be used to determine a temperature threshold value to distinguish between mouth breathing and nasal breathing.

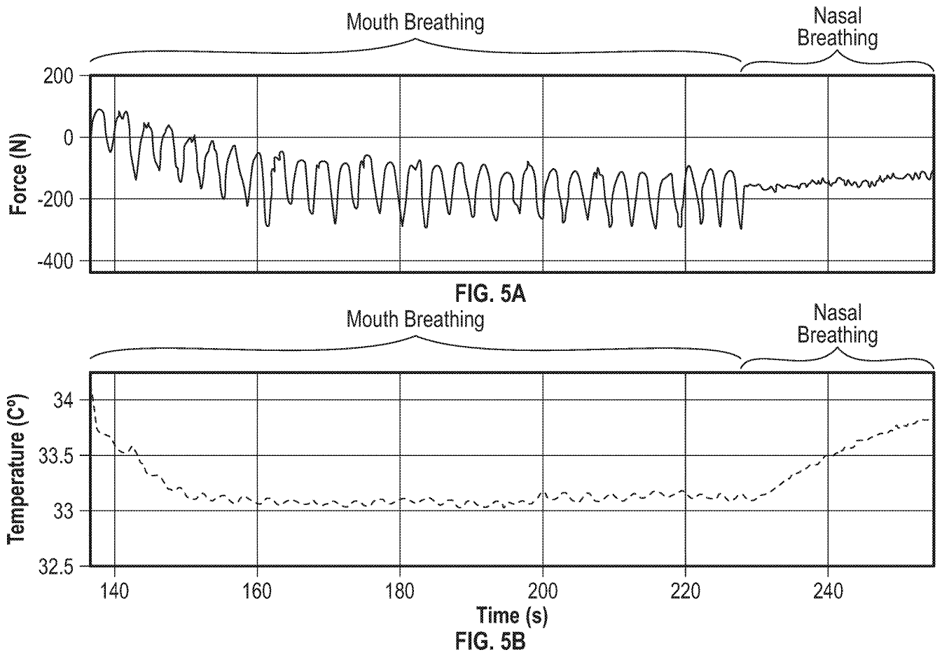

FIGS. 5A and 5B are graphs illustrating force data (FIG. 5A) and temperature data (FIG. 5B) measured by an intraoral monitoring device during mouth breathing and nasal breathing, in accordance with embodiments of the present technology. Referring first to FIG. 5A, the force data exhibits larger oscillations during mouth breathing (due to larger temperature changes) and smaller oscillations during nasal breathing (due to smaller temperature changes), thus allowing the two different types of breathing to be differentiated from each other. Moreover, during mouth breathing, the force oscillations for individual breaths can be distinguished, thus allowing for monitoring of mouth breathing patterns such as respiratory rate, depth, rhythm, etc. As shown in FIG. 5B, the temperature data exhibits a lower average temperature during mouth breathing (due to more air exchange with the cooler ambient environment) and a higher average temperature during nasal breathing (due to less air exchange with the ambient environment), which can also be used to distinguish between mouth and nasal breathing.

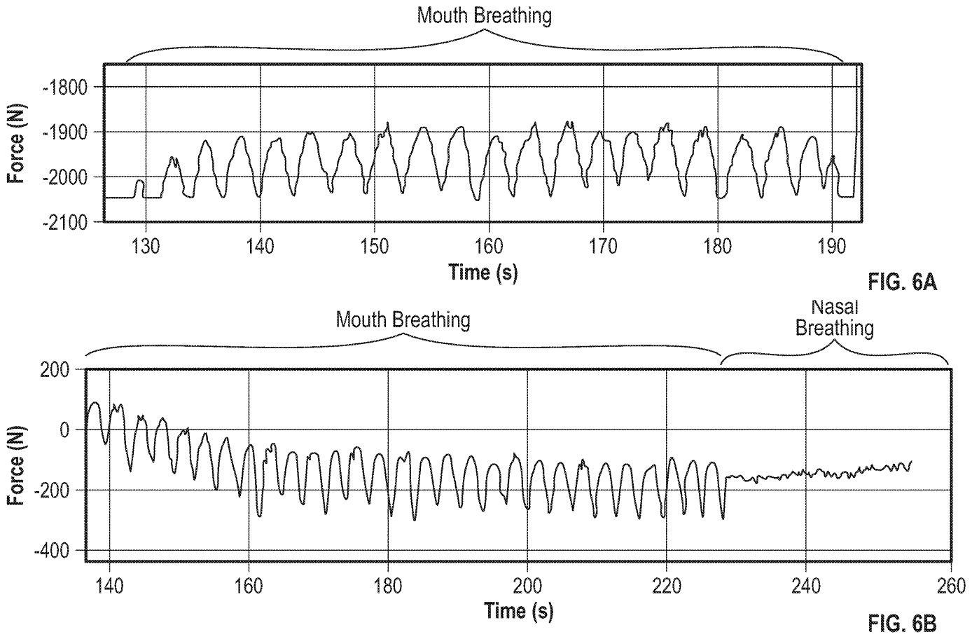

FIGS. 6A and 6B are graphs illustrating force data as measured by intraoral monitoring devices fabricated using additive manufacturing (FIG. 6A) and thermoforming (FIG. 6B), in accordance with embodiments of the present technology. As shown, both intraoral monitoring devices are sensitive to changes in force associated with respiration, thus indicating that this approach is applicable to devices produced using different manufacturing techniques.

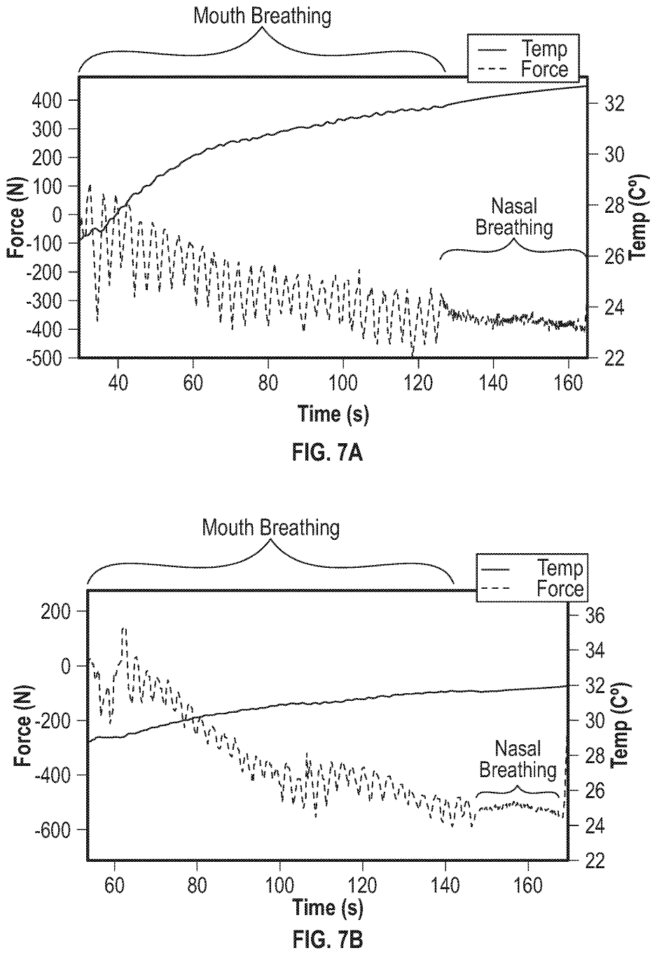

FIGS. 7A and 7B are graphs illustrating force and temperature data measured by intraoral monitoring devices having a thermosensitive material (e.g., a thermally conductive epoxy-FIG. 7A) and a non-thermosensitive material (e.g., a non-thermally conductive epoxy—FIG. 7B), in accordance with embodiments of the present technology. Referring first to FIG. 7A, the thermosensitive material may encapsulate a force sensor of the intraoral monitoring device, e.g., as described in connection with FIGS. 4A and 4B. The thermosensitive material may enhance the sensitivity of the device to temperature changes within the oral cavity. For instance, the thermosensitive material may exhibit greater deformation in response to temperature changes, thereby providing for larger, more distinguishable changes in force with respiration, as shown in FIG. 7A. In contrast, the non-thermosensitive material may exhibit less deformation in response to temperature changes, thereby providing smaller, less distinguishable changes in force with respiration, as shown in FIG. 7B. As such, the material selected for encapsulating the sensor may be selected based on its thermal conductivity. Generally, higher thermal conductivity may result in greater and/or faster deformation due to temperature changes. A material with an optimal level of thermal conductivity (among other properties) may be selected to have a thermal conductivity high enough such that the material deforms sufficiently and quickly in response to temperature changes, but not so high that the material deforms beyond an upper threshold (e.g., to prevent damage to the sensor and/or prevent disengagement of the sensor from encapsulation, to not be overly sensitive so as to make it difficult to accurately monitor and interpret gradual temperature variations, to prevent compromise to structural integrity of the material and/or the sensor module as a whole, etc.).

In some embodiments, the systems and devices herein can use a pressure sensor for monitoring a subject's respiration, additionally or alternatively to using a force sensor and/or a temperature sensor. The pressure sensor may be configured to directly measure air flow dynamics caused by a subject's breathing. The use of a pressure sensor may be beneficial, for example, in providing a more direct measurement of respiration. Moreover, a pressure sensor may be advantageous for monitoring nasal breathing patterns, which may be challenging to detect with sufficient sensitivity for other sensor types. However, in some instances, the pressure sensor may benefit from being encapsulated, e.g., to maintain or improve functionality in the humid and warm conditions of the oral cavity. This approach may provide more reliable operation of the pressure sensor in an in vivo environment and/or can improve biocompatibility, thereby enhancing the suitability of such systems for long-term intraoral health monitoring applications.

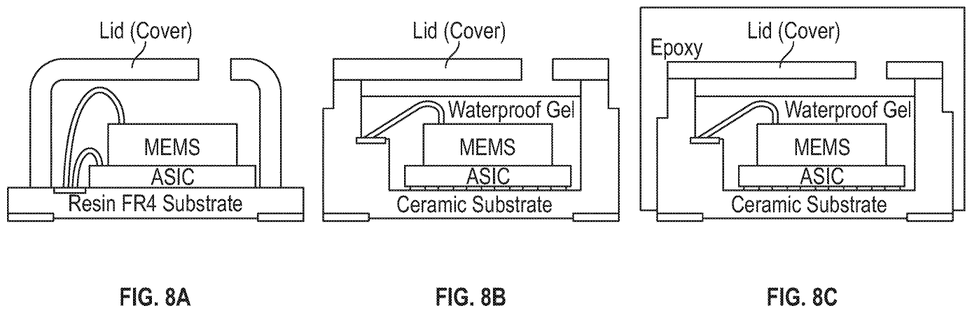

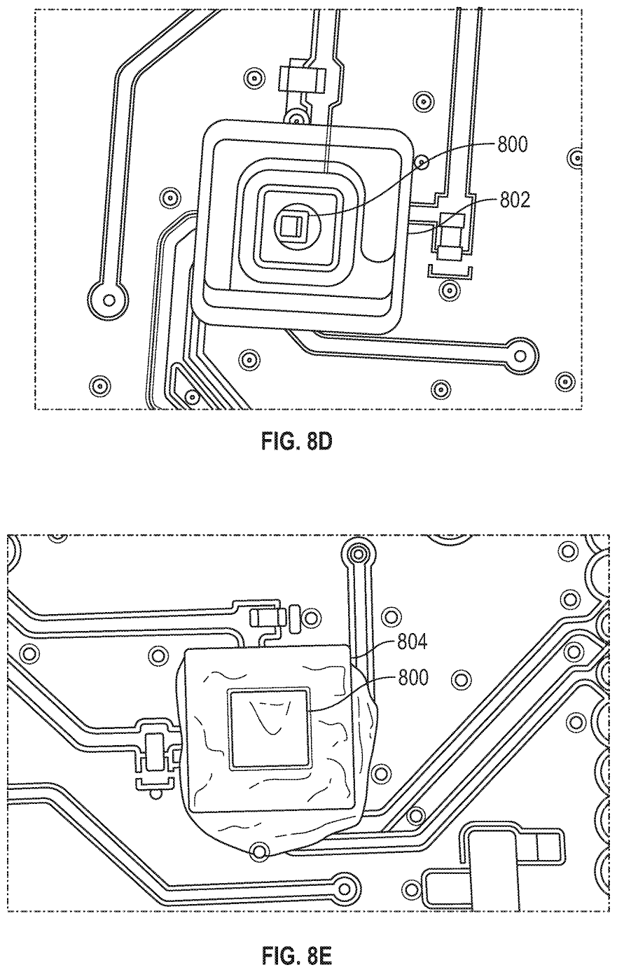

FIG. 8A is a side cross-sectional view of a pressure sensor, in accordance with embodiments of the present technology. The pressure sensor can include a MEMS-based sensing unit, which may be coupled to electrical circuitry (e.g., an application-specific integrated circuit (ASIC)) and a substrate (e.g., a resin FR4 substrate). The pressure sensor can be protected by a rigid lid or cover with a vent. The lid or cover may provide structural robustness, while the vent can allow ambient pressure to reach the MEMS-based sensing unit for accurate pressure measurements. However, these types of pressure sensors may benefit from improved water resistance, e.g., saliva and other fluids in the oral cavity may penetrate into the sensing unit, which may damage or disable the pressure sensor.

FIG. 8B is a side cross-sectional view of a pressure sensor including a gel, in accordance with embodiments of the present technology. The pressure sensor may be generally similar to the pressure sensor of FIG. 8A, e.g., the pressure sensor includes a MEMS-based sensing unit, electrical circuitry (e.g., an ASIC), and a substrate (e.g., a ceramic substrate). In some embodiments, the gel may be used to prevent unwanted exposure to fluids, thereby rendering the pressure sensor water resistant or waterproof. For instance, the gel may be disposed around the MEMS-based sensing unit and ASIC. The gel can shield the MEMS-based sensing unit from moisture, while also maintaining sensitivity and measurement accuracy due to factory calibration.

FIG. 8C is a side cross-sectional view of a pressure sensor including a gel and a biocompatible coating, in accordance with embodiments of the present technology. The pressure sensor may be generally similar to the pressure sensor of FIG. 8B, except that the pressure sensor is encapsulated in the biocompatible coating. In some embodiments, the biocompatible coating includes an epoxy (e.g., a medical grade epoxy) or silicone. This may be advantageous, e.g., for improved biocompatibility and/or water resistance.

FIGS. 8D and 8E illustrate a process for encapsulating a pressure sensor 800, in accordance with embodiments of the present technology. For instance, FIG. 8D illustrates the pressure sensor 800 surrounded by a mold 802. The mold 802 can be a customized mold that is placed around the pressure sensor 800, e.g., to control the thickness and uniformity of the coating layer. Then, as shown in FIG. 8E, a biocompatible material 804 can be disposed into the mold 802 to at least partially or fully cover the pressure sensor 800. To improve adhesion and/or reduce air gaps, the biocompatible material 804 can be disposed into the mold 802 under low-pressure and/or vacuum conditions, which may increase contact between the biocompatible material 804 and a gel filling (e.g., the waterproof gel of FIG. 8B) inside the pressure sensor 800.

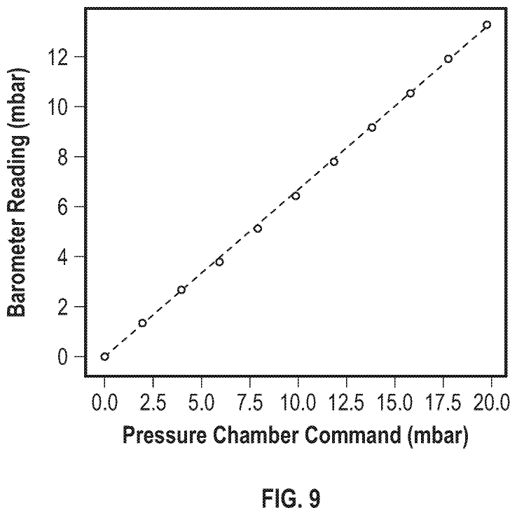

FIG. 9 is a graph illustrating validation results for the encapsulated pressure sensor 800 of FIGS. 8D and 8E, in accordance with embodiments of the present technology. As depicted in FIG. 9, when the encapsulated pressure sensor 800 was placed in a controlled pressure chamber, the pressure measurements of the pressure sensor 800 exhibited a strong correlation with pressure changes applied by the pressure chamber, demonstrating both sensitivity and accuracy. These results confirm the feasibility of the encapsulated pressure sensors described herein for reliable intraoral pressure measurements.

The encapsulated pressure sensors described herein can be incorporated into any of the intraoral monitoring systems and devices described herein (e.g., the systems and devices described above in connection with FIGS. 1-3C). When incorporated, the pressure sensor can capture subtle intraoral pressure changes associated with both mouth and nasal breathing, enabling the measurement of respiratory metrics such as respiratory rate, depth, and/or rhythm. In some embodiments, the pressure sensor may work in tandem with the force sensors and/or temperature sensors described herein, thereby forming a multi-sensor device that provides a comprehensive and detailed analysis of the subject's respiratory patterns. This combined sensing approach enhances the capability for continuous, non-invasive respiratory monitoring, making it well-suited for applications in health monitoring and sleep analysis, for example.

In some embodiments, the systems and devices herein can use a motion sensor for monitoring a subject's respiration, additionally or alternatively to using a force sensor, temperature sensor, and/or pressure sensor. For instance, a motion sensor may be part of a sensor assembly of an intraoral monitoring device, and the motion sensor can be configured to measure motion data within the subject's oral cavity. The motion data may correspond to the subject's respiration, e.g., the pattern (e.g., waveform) of the motion data may correlate to the respiration pattern; periods of increased motion (e.g., greater average amplitude) may correlate to breathing, while periods of decreased motion (e.g., lower average amplitude) may correlate to pauses between breaths; etc.

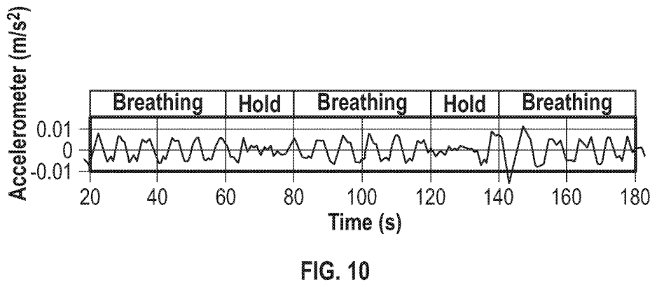

FIG. 10 is a graph illustrating acceleration data as measured by an IMU of an intraoral monitoring device, in accordance with embodiments of the present technology. As shown, the IMU effectively captured subtle acceleration signals generated by respiratory movements, thus enabling the reconstruction of respiration waveforms. Moreover, the IMU captured the reduction in the acceleration amplitude during times when the subject was holding their breath, thus indicating the sensitivity of the IMU to respiratory activity. Accordingly, the motion data can be used to determine breathing patterns (e.g., detection of irregular breathing patterns), respiratory rate, and/or other respiratory parameters of the subject. Optionally, techniques such as advanced signal processing and/or machine learning models may be used to further analyze the motion data for estimating respiratory parameters.

In some embodiments, the intraoral monitoring devices and systems described herein can include a motion sensor for measuring head ballistocardiography (BCG) data, as will be described further in Section I.C below. The head BCG data may include a component that is indicative of one or more respiratory parameters. For instance, the subject's head may exhibit periodic movements (e.g., head bobbing) as the subject breathes, which may manifest as a low frequency oscillatory component in the BCG data. Based on these periodic movements, one or more respiratory parameters may be determined, such as respiratory rate, depth, rhythm, etc.

In some embodiments, the intraoral monitoring devices described herein can monitor and/or determine one or more respiratory parameters based on orofacial activity, as will be described further in Section I.D below. For example, orofacial movements such as movements of the tongue, lips, mouth, jaws, cheeks, etc., may be detected using various types of sensors, such as force sensors, pressure sensors, motion sensors (e.g., IMU), contact sensors, proximity sensors, mechanical sensors (e.g., strain gauges), optical sensors, capacitive sensors, etc. The orofacial movements can then be analyzed to detect patterns, trends, changes, etc., which in turn can be correlated to respiratory parameters such as respiratory rate, depth, rhythm, etc.

### B. Bruxism Monitoring

In some embodiments, the intraoral monitoring devices and systems described herein (e.g., the systems and devices described above in connection with FIGS. 1-3C) can be used to monitor a subject's teeth, such as for bruxism. Bruxism, involving teeth grinding and/or jaw clenching, can result in tooth damage, jaw pain, headaches, temporomandibular disorders, and more. Unfortunately, a subject may have difficulty identifying and/or detecting bruxism because it can occur while the subject is sleeping and/or unaware.

In some embodiments, the systems and devices herein monitor for bruxism using one or more sensors. For instance, bruxism may be detected based on force data, pressure data, motion data, and/or any other suitable sensor data. In some embodiments, bruxism events are detected when force and/or motion data exceed a predetermined threshold value. The predetermined threshold value may be based on historical bruxism data of the subject, and/or may be based on historical bruxism data from a plurality of subjects. Further, types of bruxism events (e.g., teeth grinding vs. jaw clenching) may be differentiated based on patterns (e.g., waveforms), amplitudes, durations, etc., of the force and/or motion data.

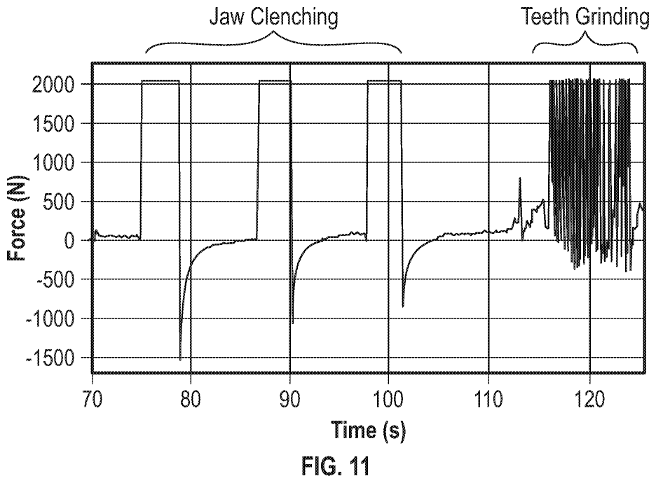

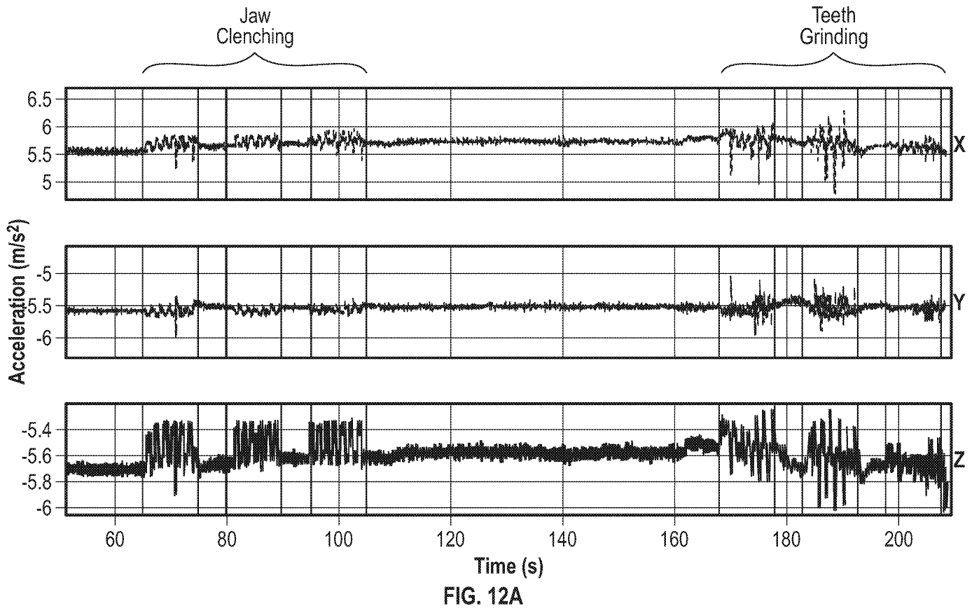

FIG. 11 is a graph illustrating force data measured by a force sensor of an intraoral monitoring device during simulated bruxism events, in accordance with embodiments of the present technology. As shown in FIG. 11, intraoral tests demonstrate that the force sensor responded sensitively to simulated bruxism events and was capable of distinguishing between jaw clenching (which produced lower frequency oscillations in force with a longer period) versus teeth grinding (which produced higher frequency oscillations with a shorter period).

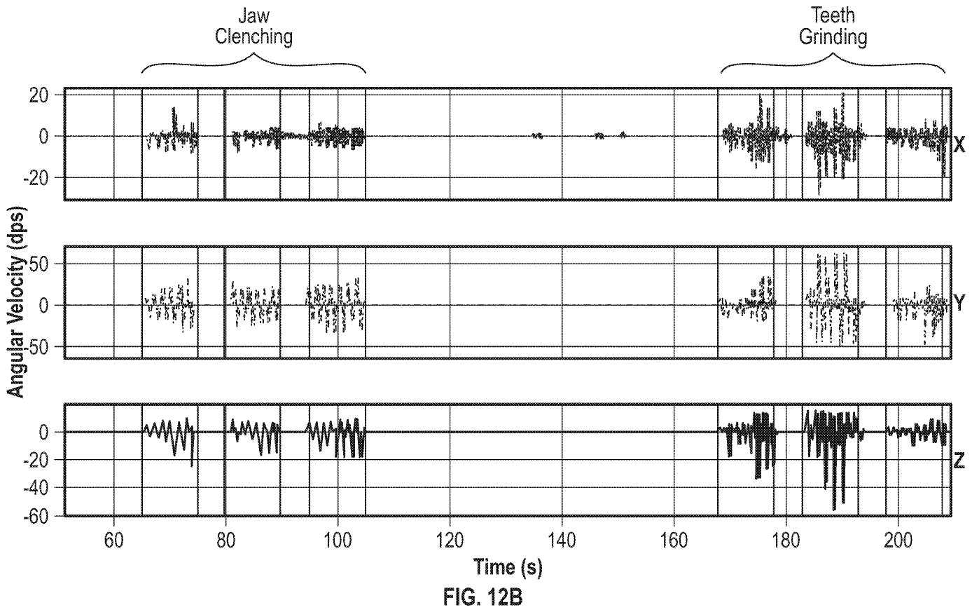

FIGS. 12A and 12B are graphs illustrating acceleration data (FIG. 12A) and angular velocity data (FIG. 12B) measured by an IMU of an intraoral monitoring device during simulated bruxism events, in accordance with embodiments of the present technology. As shown in FIGS. 12A and 12B, both jaw clenching and teeth grinding motions were clearly captured across all IMU axes. These bruxism events generated less periodic but higher-intensity acceleration and rotational signals, thus indicating the ability of the device to differentiate between physiological events based on signal characteristics. Optionally, combining the IMU with other sensors, such as force sensors for bite force measurement, may provide a more comprehensive assessment of sleep-related activities, including sleep apnea, bruxism severity, and/or other nocturnal behaviors. These results highlight the versatility of the intraoral monitoring devices described herein for multimodal physiological monitoring, thus making it a promising tool for advanced health and sleep analysis.

In some embodiments, the intraoral monitoring devices described herein can monitor and/or detect bruxism based on orofacial activity, as will be described further in Section I.D below. For example, orofacial movements such as movements of the tongue, lips, mouth, jaws, cheeks, etc., may be detected using various types of sensors, such as force sensors, pressure sensors, motion sensors (e.g., IMU), contact sensors, proximity sensors, mechanical sensors (e.g., strain gauges), optical sensors, capacitive sensors, etc. The orofacial movements can then be analyzed to detect patterns, trends, changes, etc., which in turn can be used to identify bruxism events.

### C. Cardiac Activity Monitoring

In some embodiments, the intraoral monitoring devices and systems described herein (e.g., the systems and devices described above in connection with FIGS. 1-3C) can be used to monitor a subject's cardiac activity. Cardiac function can be an important indicator of a subject's health, fitness, and/or activity levels. Examples of cardiac parameters that can be determined using the present technology include blood flow time, stroke volume, stroke volume variation, volume index, cardiac output, heart rate, heart rate variability, systolic pressure, diastolic pressure, mean arterial pressure, and/or pulse pressure. Based on the cardiac parameters, a disease or condition of the subject can be determined, such as one or more of high blood pressure, low blood pressure, arrhythmia, atrial fibrillation, tachycardia, heart failure, heart attack, stroke, congenital heart defect, or coronary artery disease. Alternatively or in combination, the cardiac parameters can be used to monitor a subject's activity level, e.g., whether the subject is resting, or has a low, moderate, or high activity level.

In some embodiments, the systems and devices herein monitor a subject's cardiac activity using ballistocardiography (BCG) techniques, such as head BCG. For example, as the heart circulates blood throughout the body, corresponding periodic movements can be observed in one or more blood vessels and surrounding tissues with each heartbeat. Blood flowing from the heart via the abdominal aorta and through the carotid arteries and other blood vessels in the head can cause periodic head movements, which may be measured by an intraoral monitoring device using a motion sensor (e.g., an accelerometer, gyroscope, and/or IMU). Although BCG can be measured from other parts of the body, BCG signals measured from the head may provide greater stability, e.g., the head may exhibit reduced movement during sleep and/or other activities compared to other body parts. Moreover, the intraoral monitoring devices and systems can be customized for the subject's oral cavity (e.g., the subject's teeth), which can ensure consistent signal acquisition from the substantially the same location over time. This consistency may allow for the accurate quantification of BCG signals for monitoring of cardiac parameters for many uses cases.

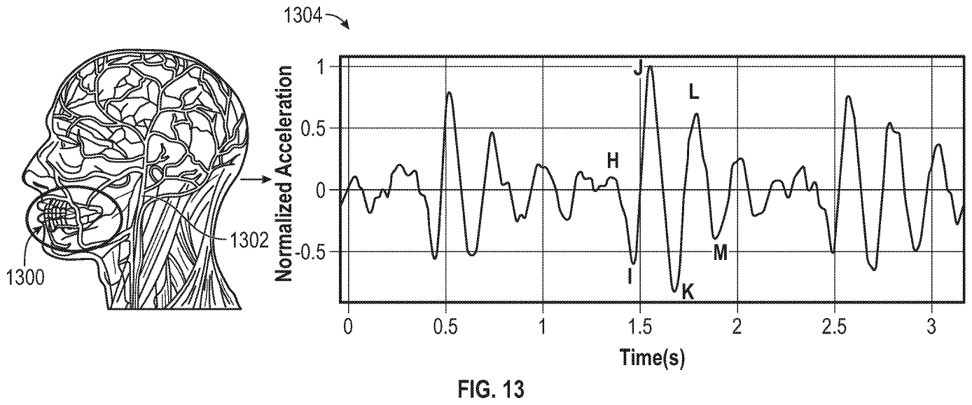

FIG. 13 illustrates monitoring of a subject's cardiac activity via head BCG, in accordance with embodiments of the present technology. In some embodiments, an intraoral monitoring device (not shown) including a motion sensor is positioned in a subject's oral cavity 1300. The intraoral monitoring device can be any of the intraoral monitoring devices described herein, such as the intraoral monitoring device 300c of FIG. 3. For instance, the intraoral monitoring device may include a motion sensor, such as a piezoelectric sensor (e.g., a polyvinylidene fluoride (PVDF) sensor), electromechanical film (EMFI) sensor, and/or IMU. The motion sensor can be configured to detect motion data of the head associated with blood flow through one or more vessels 1302 of the subject's head. In some embodiments, the motion data includes acceleration data, and the acceleration data may include one or more of vertical, sagittal, or frontal components (e.g., as measured with respect to the vertical, sagittal, and frontal axes of the subject). The vertical component of the acceleration data may correlate to the subject's cardiac cycle (e.g., to movement of blood from the heart through the carotid arteries to the head).

The workflow can further include determining a cardiac parameter based on the motion data. For instance, the motion data can include a pattern (e.g., waveform) that correlates to different stages of the cardiac cycle. As shown in graph 1304, for example, the waveform of the acceleration data may have one or more identifiable recurring peaks/troughs (labeled H-M in FIG. 13), each marker representing a different stage of the cardiac cycle. By using the pattern as a proxy for the subject's cardiac cycle, one or more cardiac parameters can be determined. For instance, head movements may begin when the left ventricle ejects blood, and the duration of these head movements may correspond with left ventricular ejection time (LVET). As shown in the graph 1304, the waveform between markers I and J (“I-J wave”) can correspond to the subject's left ventricular ejection, with the peak of the I-J wave occurring during LVET, and the duration of the I-J wave can correspond to the duration of the subject's left ventricular ejection. For instance, if the head movement acceleration reaches approximately 10 milligravity and the LVET is estimated to be around 300 ms, the corresponding displacement of the head is approximately 0.5 mm. In some embodiments, the subject's heart rate can be determined by dividing the total number of I-J wave instances in the motion data by time elapsed. Furthermore, the amplitude of the J marker can have a linear relationship with the subject's stroke volume. Based on the J-wave amplitude, cardiac output may also be predicted, since cardiac output is the product of stroke volume and heart rate.

Many other cardiac parameters may be determined based on the BCG data, e.g., blood flow time, stroke volume variation, volume index, cardiac output, heart rate variability, systolic pressure, diastolic pressure, mean arterial pressure, and/or pulse pressure. These cardiac parameters may be compared to clinical standards and/or subject-specific thresholds, and thus may be useful for managing conditions such as heart failure and myocardial infarction, among others.

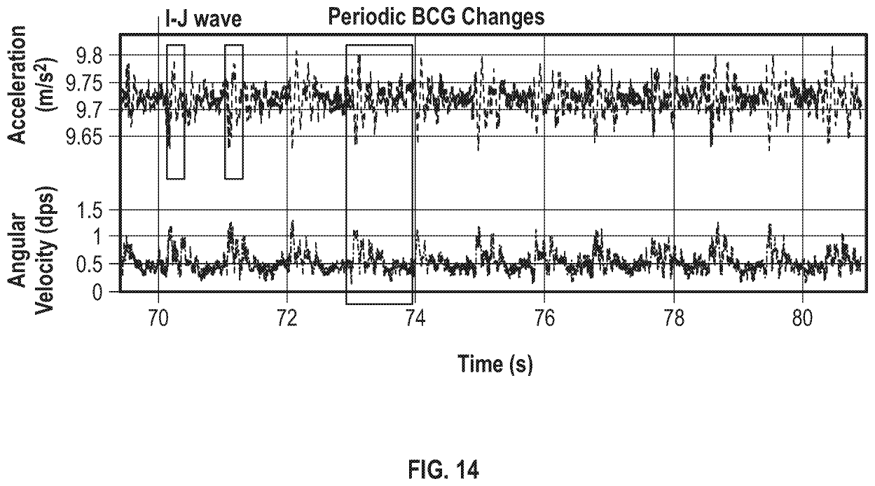

FIG. 14 is a graph illustrating example head BCG data measured by a motion sensor of an intraoral monitoring device, in accordance with embodiments of the present technology. In some embodiments, the intraoral monitoring device is a thermoformed dental appliance with a monitoring unit having an IMU. The IMU may be positioned between a palatal and lingual area of the subject, and the IMU may be configured to detect motion data associated with blood flow in the subject's head. In the illustrated example, head BCG data was captured while the subject was lying down with minimal movement to simulate a typical sleep environment. The subject simultaneously used an FDA-approved sleep monitoring system to record a two-lead arm electrocardiogram (ECG) and thoracic respiratory effort signals, for validation of the BCG-based approach. In the graph, normalized accelerometer (top) and gyroscope (bottom) data are shown. The normalized accelerometer signal captured the I-J wave, as well as further residual oscillations (“periodic BCG changes”) associated with cardiac-induced head movement. During LVET, the acceleration of the subject's head was measured to be 10-15 milligravity. Further, the gyroscope also captured periodic BCG changes associated with cardiac-induced head rotation.

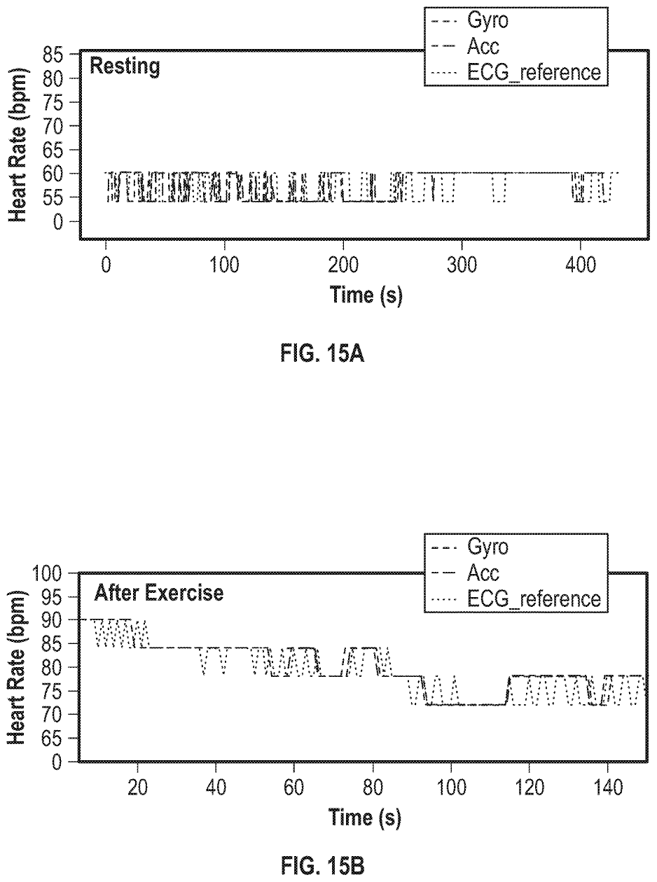

FIGS. 15A and 15B are graphs illustrating heart rate data estimated from head BCG determined by an intraoral monitoring device during rest (FIG. 15A) and after exercise (FIG. 15B), in accordance with embodiments of the present technology. The heart rate data may be determined based on motion data collected by an intraoral monitoring device, e.g., as described above. In some embodiments, the heart rate data (e.g., pulse waveforms, estimated heart rate) is determined from accelerometer and/or gyroscope data using a data processing algorithm. As shown in FIG. 15A, the heart rate derived from BCG (e.g., the accelerometer (“Acc”) and gyroscope (“Gyro”) data) is strongly correlated with a reference heart rate calculated from ECG signals, thereby confirming the reliability of the BCG-based approach. Moreover, FIG. 15B shows that elevated heart rate data taken after physical exercise was also accurately and clearly captured using the BCG-based approach, and thus can be used to determine a subject's activity level. These results demonstrate the ability of the intraoral monitoring device to accurately detect head BCG and extract cardiac parameters such as heart rate, heart rate variability, etc. The findings underscore the potential of the intraoral monitoring device for continuous health and sleep monitoring applications, including sleep staging and the detection of cardiac irregularities and sleep-related disorders.

### D. Orofacial Activity Monitoring

In some embodiments, the intraoral monitoring devices and systems described herein (e.g., the systems and devices described above in connection with FIGS. 1-3C) can be used to monitor one or more orofacial activities of a subject. For example, the orofacial activities can include swallowing and/or saliva-related behaviors, such as swallowing frequency, swallowing type (e.g., dry swallowing versus swallowing of fluids/food), swallowing pattern (e.g., isolated swallowing versus repeated swallowing, frequent micro-swallows), saliva pooling, drooling episodes, oral dryness cycles, lip sucking, and/or expectorating. As another example, the orofacial activities can include tongue behaviors, such as tongue-to-palate contact, tongue-to-teeth contact, tongue thrusts, tongue posture, and/or tongue movements (e.g., lateral movements, anterior-posterior movements). In a further example, the orofacial activities can include jaw behaviors, such as bruxism, jaw position, jaw opening/closing cycles, jaw movements (e.g., lateral movements, anterior-posterior movements, mandibular micro-movements, rhythmic movements), and/or jaw clenching (e.g., with or without grinding). In yet another example, the orofacial activities can include lip and/or cheek-related behaviors, such as lip posture (e.g., whether the lips are sealed or opened), cheek pressure (e.g., during certain sleep positions), and/or interactions of the lips and/or cheeks with the dental appliance. As a further example, the orofacial activities can include coughing, throat clearing, yawning, snoring, speaking, breathing, breath holding, and/or gasping.

Orofacial activities may be monitored using one or more sensors that detect orofacial movements, such as movements of the tongue, lips, mouth, jaws, cheeks, etc. Examples of sensors that may be used to detect orofacial movements include force sensors, pressure sensors, temperature sensors, motion sensors, humidity sensors, proximity sensors, optical sensors, contact sensors, and suitable combinations thereof.

For instance, force and/or pressure sensors may be used to monitor swallowing and/or saliva-related behaviors. In some embodiments, force and/or pressure data is analyzed to detect waveforms, patterns, etc., that are associated with swallowing events. This information may also be used to distinguish between different types of swallowing events, e.g., dry swallowing versus swallowing of saliva versus swallowing of fluids/food. Force and/or pressure sensors may also be used to monitor the location and/or posture of the tongue, e.g., changes in pressure may correlate to whether the tongue is in contact with the palate or not, whether the tongue is obstructing the airway (e.g., during obstructive sleep apnea) or not, etc. Force and/or pressure sensors may also be used to detect motions (e.g., vibrations) that occur due to snoring, coughing, bruxism, clenching, etc.

As another example, temperature and/or humidity sensors may be used to monitor swallowing and/or saliva-related behaviors. In some embodiments, changes in the temperature and/or humidity of the oral cavity (e.g., temporary cooling) may be correlated to swallowing events. For example, a temperature sensor may detect a drop in measured local temperature due to swallowing and/or a humidity sensor may detect a change in humidity due to swallowing.

Detected patterns and/or shifts correlated to saliva-related behaviors (e.g., detected by temperature, humidity, force, and/or pressure sensors) over time may provide insights into saliva production, oral dryness cycles, and/or drooling episodes. By tracking these sensor signals (e.g., for an extended period of time), the system can offer a detailed assessment of swallowing frequency, types of swallowing, and the overall hydration status of the oral cavity, which may be valuable for monitoring hydration, salivary gland function, or diagnosing conditions like xerostomia or hypersalivation. This approach allows for non-invasive, continuous monitoring of oral behaviors that are relevant to both oral and general health.

In yet another example, motion sensors may be used to detect orofacial movements indicative of various orofacial activities, such as swallowing, tongue movements, vibrations due to snoring, vocalizations (e.g., speaking, grunting, coughing), breathing (e.g., mouth breathing versus nasal breathing), etc.

In some embodiments, different sensor types may be used to monitor a particular orofacial activity. For instance, tongue location may be monitored using optical sensors, contact sensors, and/or proximity sensors. Mouth breathing may be monitoring using mechanical sensors (e.g., a strain gauge, a structure that deflects in response to movement), optical sensors (e.g., LEDs), and/or capacitive sensors. Saliva levels may be detected using optical sensors (e.g., based on reflection of light toward the cheek), capacitive sensors, impedance sensors, and/or force and/or pressure sensors (e.g., based on material swelling of a dental appliance).

In some embodiments, a plurality of sensors are used to monitor a particular orofacial activity. For example, jaw articulation may be monitored using a sensor located on the upper jaw and a sensor located on the lower jaw, where the sensors are configured to measure the distance between jaws (e.g., optical sensors, magnetic sensors such as Hall effect sensors).

The location of the sensors may be selected based on the type of orofacial activity to be monitored. For instance, temperature sensors may be located near the anterior portion of the oral cavity to facilitate detection of temperature changes due to breathing, jaw opening, etc. Force and/or pressure sensors may be located at regions of the oral cavity where force and/or pressure is expected to be applied, such as the buccal, lingual, occlusal, and/or palatal areas. Motion sensors may be located near structures of the oral cavity that are expected to exhibit movement, e.g., proximate to the tongue, lips, and/or jaws.

In some embodiments, the intraoral monitoring devices and systems herein are configured to distinguish between different types of orofacial activities. Certain types of orofacial activities may be associated with certain signal characteristics in the sensor data (e.g., frequency, amplitude, period, waveforms, patterns). For instance, snoring may be characterized by a higher motion frequency (e.g., 20-80 Hz) and a periodic motion pattern. Swallowing and coughing may be characterized by a short motion duration with a relatively large motion amplitude. Thus, the sensor data can be analyzed to detect the presence of various signal characteristics and the signal characteristics can be correlated to different orofacial activities, thereby allowing the incidence and timing of orofacial activities to be detected. Optionally, signal characteristics of multiple sensor types can be compared to each other to determine the type of detected orofacial activity, e.g., the motion patterns produced by snoring may correlate to the changes in temperature, airflow, pressure, etc., associated with the respiration cycle (e.g., inhalation and exhalation).

The associations between signal characteristics and particular types of orofacial activities can be determined in various ways. For instance, sensor data can be obtained from a representative patient population while those patients are performing certain orofacial activities. The sensor data can then be analyzed using suitable data analysis techniques (e.g., statistical analysis, Fourier analysis, principal component analysis) to identify signal characteristics that are indicative of various orofacial activities. Alternatively or in addition, machine learning algorithms can be trained to detect the incidence of orofacial activities from sensor data.

In some embodiments, the intraoral monitoring devices and systems herein are configured to determine a physiological parameter and/or health condition based on the detected orofacial activities. For instance, changes in the frequency, timing, and/or pattern of swallowing events may be correlated to sleep parameters, such as whether the subject is asleep or awake, the current sleep stage, etc., and thus may be used to identify sleep quality and/or sleep-related disorders (e.g., sleep disruptions, sleep apnea). Saliva levels may be inferred from the frequency swallowing as well as the type of swallowing (e.g. dry versus wet swallowing), and thus may be used to detect saliva-related conditions (e.g., dry mouth, hypersalivation). As another example, movement patterns and/or applied forces may be used to detect the frequency and location of bruxism.

In some embodiments, the intraoral monitoring devices and systems herein are configured to determine one or more root causes for an orofacial activity that is associated with a particular physiological parameter and/or health condition. The determination of the root cause can be based on the timing of one or more detected orofacial activities, such as the time point(s) when the orofacial activity occurs and/or the duration of the orofacial activity. In some embodiments, the timing of a detected orofacial activity is correlated to the timing of other events (e.g., other orofacial activities occurring at substantially the same time), and the correlation is used to determine the root cause. The determined root cause may be used to diagnose the subject with a disease or condition.

In some embodiments, the intraoral monitoring devices and systems herein can generate treatment recommendations based on the determined physiological parameters, health conditions, and/or root causes. The treatment recommendations can be configured to prevent and/or treat health risks that are associated with the detected orofacial activities and/or the underlying root causes. For example, lower saliva levels may be correlated with an increased risk of caries; thus, the treatment recommendations may include increasing fluid consumption (e.g., the subject may be reminded to drink water more frequently and/or to drink water at specific times, such as before bed).

Examples of root causes that may be associated with orofacial activities include: neurological and/or sleep-related disorders, such as parasomnia, movement disorders, sleep deprivation, and obstructive sleep apnea; psychological and/or emotional factors such as stress and anxiety; dental and/or oral conditions such as malocclusion, temporomandibular joint dysfunction, and oral muscle tension; respiratory issues such as airway obstruction, nasal obstruction, and postnasal drip; gastrointestinal and/or internal conditions such as acid/gastroesophageal reflux; and dermatological and/or immune-related conditions such as nocturnal pruritic and fever.

Examples of orofacial activities that may be monitored and correlated to root causes include: dental and/or oral motor behaviors such as bruxism, chewing, tooth tapping, tongue clicking, swallowing, and lip sucking; sleep-related vocalizations such as sleep talking and grunting; head and/or facial movements such as head motion, head rubbing, and head scratching; and respiratory behaviors such as mouth breathing.

Examples of health risks that may be associated with orofacial activities and the correlated root causes include: dental and/or oral issues such as dental damage, dental misalignment, dry mouth, bad breath, cavities, and speech difficulties; jaw and/or musculoskeletal issues such as temporomandibular joint disorders and jaw pain; sleep quality and/or arousal effects such as sleep disruption, poor sleep quality, sleep fragmentation, reduced sleep duration, prolonged arousal, and daytime fatigue; dermatological and/or soft tissue effects such as skin lesions and throat inflammation; and gastrointestinal issues such as esophageal irritation.

Table 1 below provides representative examples of correlations between root causes and orofacial activities. Table 2 below provides representative examples of correlations between orofacial activities and health risks.

TABLE 1
Correlations Between Root Causes and Orofacial Activities

| Root Cause | Orofacial Activities |
|---|---|
| Obstructive sleep apnea | Bruxism, chewing, tooth tapping, swallowing |
| Parasomnia | Head movement |
| Movement disorder | Head movement |
| Sleep deprivation | Sleep talking |
| Stress, anxiety | Bruxism, chewing, tooth tapping, lip sucking, |
| | sleep talking, grunting, head rubbing, head |
| | scratching |
| Malocclusion | Bruxism, chewing, tooth tapping, lip sucking |
| Temporomandibular joint dysfunction | Tongue clicking |
| Oral muscle tension | Tongue clicking |
| Airway obstruction | Swallowing, mouth breathing |
| Nasal obstruction | Mouth breathing |
| Postnasal drip | Grunting |
| Acid/gastroesophageal reflux | Bruxism, chewing, tooth tapping, swallowing |
| Nocturnal pruritis | Sleep talking, head rubbing, head scratching |
| Fever | Sleep talking |

TABLE 2
Correlations Between Orofacial Activities and Health Risks

| Orofacial Activities | Health Risks |
|---|---|
| Bruxism, chewing, | Dental damage, temporomandibular joint |
| tooth tapping | disorders, jaw pain, sleep disruption, poor |
| | sleep quality |
| Tongue clicking | Dental damage, speech difficulties, jaw pain |
| Swallowing | Prolonged arousal, esophageal irritation |
| Lip sucking | Dental misalignment, jaw pain |
| Sleep talking | Daytime fatigue |
| Grunting | Sleep fragmentation, reduced sleep duration, |
| | throat inflammation |
| Head movement | Sleep fragmentation, reduced sleep duration |
| Head rubbing, | Sleep disruption, poor sleep quality, skin |
| head scratching | lesions |
| Mouth breathing | Dry mouth, bad breath, cavities, daytime |
| | fatigue |

### E. Associated Methods

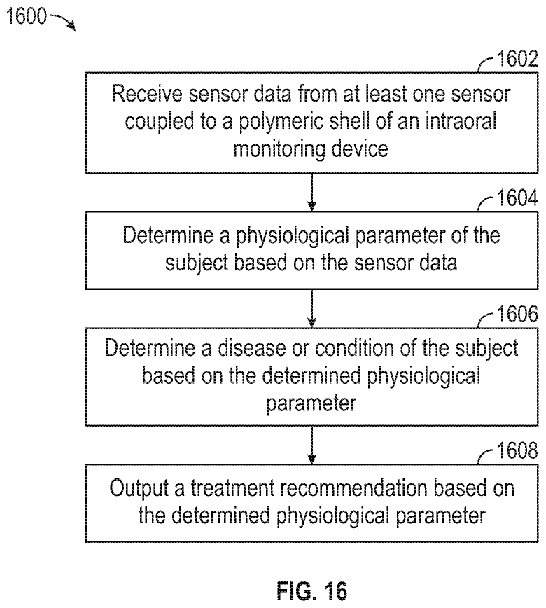

FIG. 16 is a flow diagram illustrating a method 1600 for intraoral monitoring, in accordance with embodiments of the present technology. The method 1600 may be used for monitoring of any of the physiological parameters and/or orofacial activities described herein, e.g., in Sections I.A-I.D above. In some embodiments, some or all of the processes described with respect to the method 1600 are implemented as computer-readable instructions (e.g., program code) that are configured to be executed by one or more processors of a computing device (e.g., an electronics assembly of a intraoral monitoring device, a mobile device, laptop, personal computer, workstation, remote server, smart appliance case). The computing device may be part of a virtual dental care system as described in, e.g., U.S. Patent Application Publication No. 2022/0023003. The method 1600 can be performed using any of the systems and devices described herein.

The method 1600 can begin at block 1602 with receiving sensor data from at least one sensor coupled to a polymeric shell of an intraoral monitoring device. The intraoral monitoring device can be any of the systems or devices described herein, such as the systems and devices described in connection with FIGS. 1-3C. For example, the intraoral monitoring device can include a sensor assembly with the at least one sensor and an electronics assembly coupled to the sensor assembly. In some embodiments, the at least one sensor is a force sensor (e.g., a load cell, strain gauge, piezoelectric sensor), a pressure sensor (e.g., a MEMS-based sensor, strain gauge, piezoelectric sensor, capacitive sensor), a temperature sensor (e.g., a thermistor, a thermocouple), a motion sensor (e.g., an accelerometer, a gyroscope, an IMU), a humidity sensor, a proximity sensor, an optical sensor, a contact sensor, or any other suitable sensor. The sensor data can include force data, pressure data, temperature data, motion data, etc. For example, a force sensor can be used to generate force data associated with a deformation of the polymeric shell, where the deformation corresponds to respiration as discussed in Section I.A above. As another example, a motion sensor can be used to measure motion data associated with blood flow in the subject's head, as discussed in Section I.C above. The sensor data may be measured substantially continuously (e.g., once every 5 seconds, 10 seconds, 15 seconds, 30 seconds, 1 minute, 2 minutes, 5 minutes, 10 minutes, 15 minutes, 30 minutes, etc.) or for prescribed periods of time, such as while the subject is sleeping, engaging in physical activity, etc.

The method 1600 can continue at block 1604 with determining a physiological parameter of the subject based on the sensor data. The physiological parameter can be any parameter characterizing a physiological process that occurs in and/or around the oral cavity, such as respiration, cardiac activity, bruxism, etc. For example, the physiological parameter can include a respiratory parameter, such as respiration rate, respiration rhythm, respiration depth, apnea events, hypopnea events, snoring, mouth breathing, and/or nasal breathing. As another example, the physiological parameter can include a cardiac parameter, such as flow time, stroke volume, stroke volume variation, stroke volume index, cardiac output, heart rate, heart rate variability, systolic pressure, diastolic pressure, mean arterial pressure, or pulse pressure. Other physiological parameters that may be detected include sleep parameters (e.g., sleep duration, sleep depth, sleep consistency), activity parameters (e.g., activity level, minutes of exercise, pace, speed, distance traveled), etc.

The physiological parameter can be determined from the sensor data in various ways, such as by comparing the sensor data to predetermined thresholds, by evaluating waveform characteristics (e.g., amplitude, period), etc. For example, respiration rate can be determined by evaluating the period between two neighboring peaks in a force waveform. As another example, the amplitude of a force waveform may be correlated to respiration depth. In a further example, the overall pattern of the force waveform may be correlated to a respiration pattern. As another example, mouth breathing versus nasal breathing can be determined by comparing a force amplitude to a force threshold value, where the subject is determined to be mouth breathing if the force amplitude exceeds the force threshold value, and the subject is determined to be nasal breathing if the force amplitude is below the force threshold value. In another example, heart rate can be determined by analyzing periodic patterns in a BCG waveform. Further, sensor data collected from multiple sensors may be used to determine the physiological parameter, e.g., to improve accuracy and/or sensitivity. For instance, pressure data, temperature data, motion data, and/or force data may be combined to evaluate respiratory parameters.

In some embodiments, the physiological parameter includes or is determined based on one or more orofacial activities that are monitored via the at least one sensor, e.g., as described in Section I.D above. For example, the orofacial activities can include swallowing and/or saliva-related behaviors, such as swallowing frequency, swallowing type (e.g., dry swallowing versus swallowing of fluids/food), swallowing pattern (e.g., isolated swallowing versus repeated swallowing, frequent micro-swallows), saliva pooling, drooling episodes, oral dryness cycles, lip sucking, and/or expectorating. As another example, the orofacial activities can include tongue behaviors, such as tongue-to-palate contact, tongue-to-teeth contact, tongue thrusts, tongue posture, tongue movements (e.g., lateral movements, anterior-posterior movements). In a further example, the orofacial activities can include jaw behaviors, such as bruxism, jaw position, jaw opening/closing cycles, jaw movements (e.g., lateral movements, anterior-posterior movements, mandibular micro-movements, rhythmic movements), and/or jaw clenching (e.g., with or without grinding). In yet another example, the orofacial activities can include lip and/or cheek-related behaviors, such as lip posture (e.g., whether the lips are sealed or opened), cheek pressure (e.g., during certain sleep positions), and/or interactions of the lips and/or cheeks with the dental appliance. As a further example, the orofacial activities can include coughing, throat clearing, yawning, snoring, speaking, breathing, breath holding, and/or gasping.

Optionally, the method 1600 can include outputting an indication of the determined physiological parameter. The indication can be output to a user (e.g., the subject, a clinician) via a user interface of a display of a computing device (e.g., a monitor or touch screen of a mobile device (such as a smartphone), tablet, laptop, workstation, etc.). The indication of the physiological parameter can be presented in many different formats, such as numerically (e.g., values, probabilities), textually (e.g., categorizations, descriptive assessments, notifications), and/or graphically (e.g., similar to any of the graphs described herein). For instance, the indication can be a graph of the physiological parameter over time. The physiological parameter may optionally be displayed graphically alongside sensor data. Further, the indication may be associated with a confidence level, which may be based on the amount of noise in the sensor data, the sensitivity of the sensor, etc.

In embodiments where the physiological parameter includes or is based on one or more orofacial activities, the method 1600 can include outputting an indication of the timing of the orofacial activities. For example, the user interface can display a timeline (or similar graphical element) that indicates the time points and/or periods at which certain orofacial activities were detected, and/or the duration of the orofacial activities. In embodiments where multiple types of orofacial activities are monitored, the timing information for multiple orofacial activities may be displayed on the same timeline (e.g., overlaid with each other), on separate timelines (e.g., a side-by-side view), or suitable combinations thereof. This approach can allow a user (e.g., a clinician) to identify correlations between the timing of different orofacial activities, which may be helpful for identifying a root cause of the orofacial activities, e.g., as described in Section I.D above.

The method 1600 can continue at block 1606 with determining a disease or condition of the subject based on the determined physiological parameter. For example, a respiratory parameter can be used to detect one or more of asthma, pneumonia, hyperventilation, dyspnea, bradypnea, tachypnea, sleep apnea, insomnia, hypersomnia, parasomnia, narcolepsy, sleep paralysis, or snoring. As another example, a cardiac parameter can be used to detect one or more of high blood pressure, low blood pressure, arrhythmia, atrial fibrillation, tachycardia, heart failure, heart attack, stroke, congenital heart defect, or coronary artery disease. Alternatively or additionally, the determined physiological parameter can be used to detect a subject's activity level (e.g., whether the subject is resting, or has a low, moderate, or high activity level) and/or state (e.g., whether the subject is moving, eating, sleeping, etc.).

In embodiments where the physiological parameter includes or is based on one or more orofacial activities, the process of block 1606 can include identifying a root cause for the orofacial activities, and then determining the disease or condition based on the root cause, e.g., as described in Section I.D above. In some embodiments, the root cause is identified based on the timing of one or more detected orofacial activities, such as the time point(s) and/or period(s) when the orofacial activity occurs and/or the duration of the orofacial activity. In some embodiments, the timing of a detected orofacial activity is correlated to the timing of other events (e.g., other orofacial activities occurring at substantially the same time), and the correlation is used to determine the root cause. The root cause itself may be a disease or condition, or the root cause may be used to determine a disease or condition.

In some embodiments, the disease or condition determination is determined automatically. For instance, a software algorithm may be used to determine whether the determined physiological parameter corresponds to a disease or condition, or whether the subject is at risk of developing a disease or condition. In some embodiments, the software algorithm is coupled to a repository storing historical data of the subject and/or a plurality of subjects. The physiological parameter may be compared to the historical data to determine the subject's disease, condition, and/or state.

Alternatively or in addition, the physiological parameter can be sent to a clinician. The clinician may assess the physiological parameter to determine whether the physiological parameter corresponds to a disease or condition, or whether the subject is at risk of developing a disease or condition. Optionally, the clinician may determine that the physiological parameter does not sufficiently capture the subject's current state and/or condition, and the intraoral monitoring device may be instructed to collect further sensor data.

Optionally, the method 1600 can include outputting an indication of the determined disease or condition. The indication can be output to a user (e.g., the subject, a clinician) via a user interface of a display of a computing device (e.g., a monitor or touch screen of a mobile device (such as a smartphone), tablet, laptop, workstation, etc.). The indication of the determined disease or condition can be presented in many different formats, such as numerically (e.g., scores, probabilities), textually (e.g., categorizations, descriptive assessments, notifications), and/or graphically. For instance, the indication can state the type of determined disease or condition, and, optionally, a confidence level associated with the determination. Alternatively, the indication may simply state that the subject is at risk for a particular disease or condition (and, optionally, the subject's risk level for that disease or condition).

The method 1600 can continue at block 1608 with outputting a treatment recommendation based on the determined physiological parameter. In some embodiments, the treatment recommendation may include suggested changes in the subject's activities, lifestyle, diet, nutrition, etc. For instance, the treatment recommendation may include increasing aerobic exercise for improved cardiac function. Alternatively or in combination, the treatment recommendation may include suggestions for using a treatment and/or support device, such as a mouth guard for teeth grinding, a continuous positive airway pressure machine for sleep apnea, etc. Optionally, the treatment recommendation may include instructions to schedule an in-person appointment for further diagnostics, a recommendation to seek emergency care and/or therapeutic interventions (e.g., medications, surgery), etc.

In some embodiments, the treatment recommendation is output on a display. The display can be associated with a computing device (e.g., a mobile device, personal computer, laptop, tablet, workstation). The computing device may be part of or operably coupled to the intraoral monitoring device. Alternatively or in combination, the computing device can be part of a computing system (e.g., a virtual care system) that includes one or more local client devices (e.g., subject devices and/or clinician devices) communicably coupled to a remote server (e.g., of a treatment monitoring service provider) via a communications network. In some embodiments, the computing device used to display the treatment recommendation is the same as the computing device used to perform the other processes of the method 1600, e.g., all of the processes of the method 1600 are performed by a local client device. In other embodiments, the computing device used to display the treatment recommendation is different than the computing device used to perform the other processes of the method 1600, e.g., the treatment recommendation is displayed by a local client device (e.g., mobile phone) and the other processes are performed by a remote server.

The method 1600 illustrated in FIG. 16 can be modified in many different ways. For example, the ordering of the processes shown in FIG. 16 can be varied, some of the processes of the method 1600 can be omitted, and/or the method 1600 can include additional processes not shown in FIG. 16. For instance, the processes of blocks 1606 and/or 1608 may be omitted from the method 1600. Further, the method 1600 may include performing one or more signal processing operations on the sensor data, such as denoising, cleaning, normalizing, thresholding, filtering, downsampling, etc.

### II. Methods for Sensor Encapsulation

In some embodiments, it may be advantageous to at least partially or completely encapsulate one or more components of the intraoral monitoring systems and devices herein. For instance, encapsulation can protect a sensor from unwanted fluids from entering and destroying sensor elements, and/or from humid environments interfering with signal acquisition. Encapsulation may also improve biocompatibility, e.g., by isolating the components from direct exposure to oral tissues. Any of the intraoral monitoring systems and devices described herein (e.g., in Section I above) may include one or more encapsulated components, such as monitoring units, sensor assemblies, electronic assemblies, individual sensors, etc.

Two representative techniques are described below for encapsulating a monitoring unit of an intraoral monitoring device. The first technique includes a double-layer thermoforming technique to encapsulate the monitoring unit in a sealed air cavity. The second technique includes the direct encapsulation (e.g., potting) of the monitoring unit using a biocompatible material (e.g., a biocompatible epoxy). Each of these techniques may affect how the sensors of the monitoring unit detect physiological changes in the oral cavity. These techniques can be used in thermoforming (e.g., using a thermoplastic material such as EX40) and direct fabrication processes (e.g., additive manufacturing), providing strong potential in large scale manufacturing. Further, these techniques can be customized for different subjects to enhance sensor performance and user experience. As described elsewhere herein, the intraoral monitoring devices formed using these techniques can be durable, rechargeable, and/or reusable. Further, these intraoral monitoring devices may have a relatively small form factor, e.g., compared to conventional sleep monitoring or intraoral sensing wearable devices, thereby avoiding discomfort and interference with sleep.

### A. Double-Layer Thermoforming

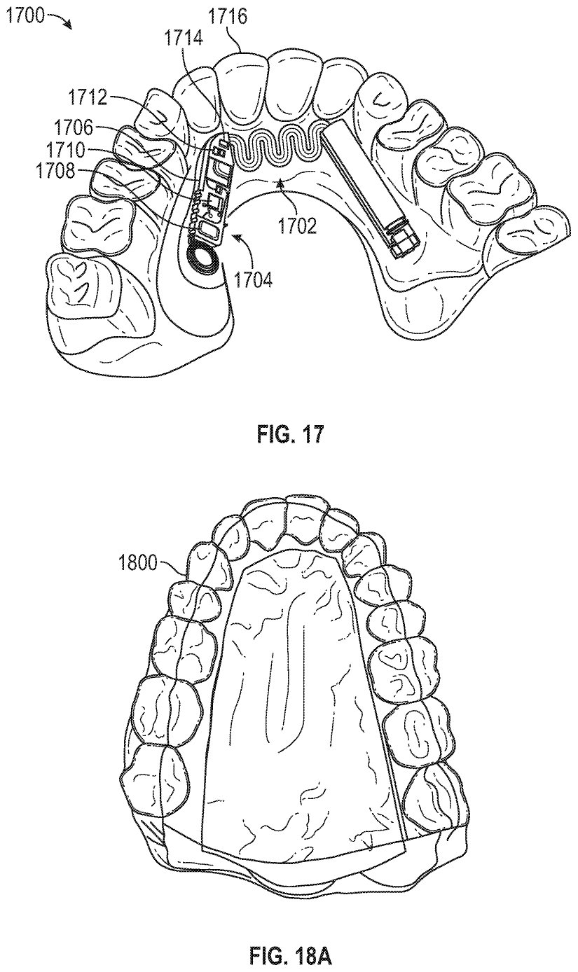

FIG. 17 illustrates an example intraoral monitoring device 1700 including a monitoring unit 1702 encapsulated using double-layer thermoforming, in accordance with embodiments of the present technology. The intraoral monitoring device 1700 and monitoring unit 1702 may be generally similar to the intraoral monitoring device 200 and monitoring unit 202 of FIGS. 2A-2E. For example, the monitoring unit 1702 can include a sensor assembly 1704 mounted on a substrate 1706. The sensor assembly 1704 can include a pressure sensor 1708, a force sensor 1710, a temperature sensor 1712, and a motion sensor 1714. Optionally, an additional pressure sensor (not shown) can be located on the back side of the substrate 1706.

The monitoring unit 1702 can be encapsulated within a sealed, water resistant (e.g., waterproof) cavity, such as a preformed pocket. The deformability of this encapsulated cavity can allow the pressure sensor 1708 to detect changes in air pressure and/or applied forces in the intraoral environment. The cavity may behave like a membrane and deform under applied force and/or pressure, thereby allowing for force and/or pressure measurements from the encapsulated sensor assembly 1704.

The force sensor 1710 can be configured to detect applied force (e.g., due to bruxism) and/or breathing (e.g., due to thermal expansion/contraction of at least a portion of the intraoral device 1700). For instance, the force sensor 1710 can detect force through the bending of the substrate 1706 on which the force sensor 1710 is mounted. In some embodiments, the force sensor 1710 is placed at or near the center of the segment of the substrate 1706 to which the force sensor 1710 is mounted to effectively capture the largest bending applied to the intraoral device 1700. In some embodiments, for the force sensor 1710 to effectively capture the bending within the oral cavity in both directions (e.g., upward and downward), it may be placed in direct contact with a shell 1716 of the intraoral device 1700 from both sides. To achieve this contact, a thick double-sided tape can be used to adhere the force sensor 1710 to the shell 1716, and the tape may be further compressed during the thermoforming process.

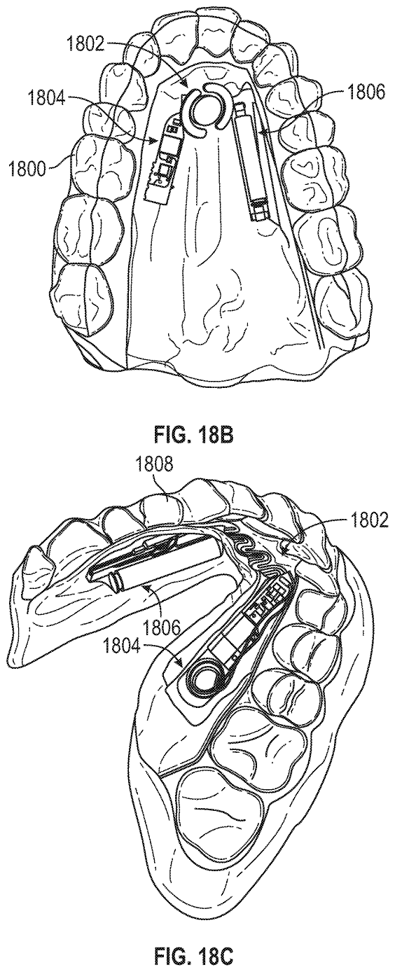

FIGS. 18A-18C illustrate an example encapsulation process for forming an intraoral monitoring device (e.g., the device 1700 of FIG. 17), in accordance with embodiments of the present technology. The encapsulation process can include a double-layer thermoforming technique. Referring first to FIG. 18A, a base layer 1800 of an intraoral monitoring device is thermoformed (e.g., over a mold—not shown). The base layer 1800 can be configured to at least partially cover a subject's palatal area and include a narrow margin on the subject's teeth area. In some embodiments, the base layer 1800 is formed from a thermoplastic material (e.g., EX15).

Referring next to FIG. 18B, a monitoring unit 1802 including a sensor assembly 1804 and an electronics assembly 1806 is positioned on the base layer 1800 of FIG. 18A. Optionally, a thick double-sided tape can be placed onto a force sensor of the sensor assembly 1804 to enhance direct contact of the force sensor with the base layer 1800, e.g., to enhance sensitivity of the force sensor.

Next, as shown in FIG. 18C, a second layer 1808 is thermoformed over the base layer 1800 (hidden in FIG. 18C) and the monitoring unit 1802. By thermoforming the second layer 1808, a sealed high-pressure cavity may be created. In some embodiments, the second layer 1808 can include a thermoplastic material (e.g., ST30). The materials for the base layer 1800 and the second layer 1808 can be selected to enhance bonding between these two layers through thermoforming.

Optionally, the double-layer thermoforming technique can include releasing pressure within the cavity to atmospheric pressure, after both layers have been thermoformed. Excessively high pressure in the cavity may be out of the detection range of the pressure sensor and/or may lead to sensing errors. Accordingly, a hole can be formed into the cavity (e.g., with a needle) to release the pressure within the cavity. The cavity may be resealed after the pressure has been released, such as with a biocompatible glue. The biocompatible glue can be a light-curable adhesive (e.g., Dymax), with the curing process including a nitrogen purge (e.g., for 50 seconds) before UV exposure to cool down the intraoral device. This cooling process may prevent overheating of the device electronics during the UV curing phase.

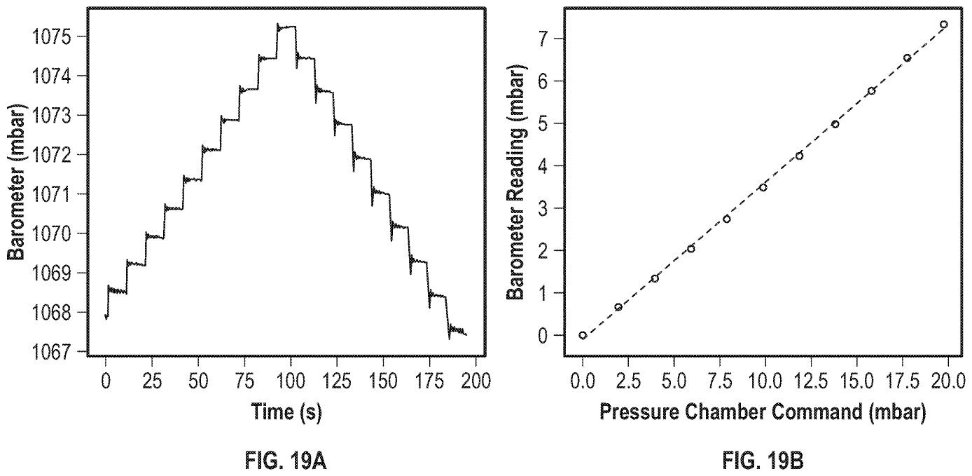

FIGS. 19A and 19B are graphs illustrating example pressure sensitivity measurements for an encapsulated pressure sensor, in accordance with embodiments of the present technology. In the illustrated example, an intraoral monitoring device with an encapsulated pressure sensor was formed according to the process of FIGS. 18A-18C. As shown in FIGS. 19A and 19B, the sensitivity of the pressure sensor was measured in a benchtop setting using a pressure chamber. The pressure change reading from the pressure sensor was compared to the actual pressure change of the pressure chamber (across different subjects, the sensitivity fell within a range from 30% to 40%).

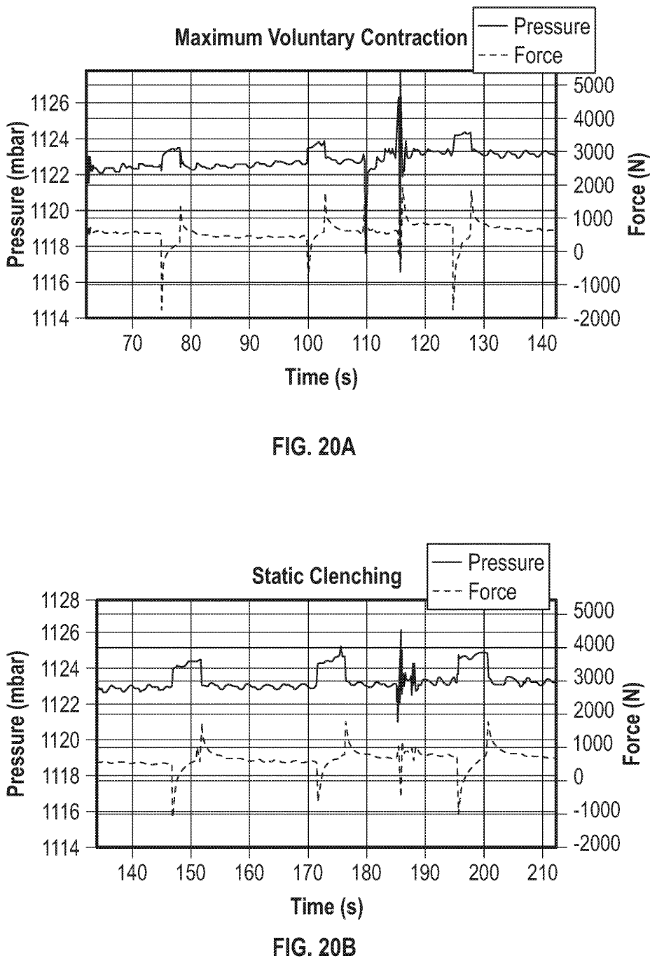

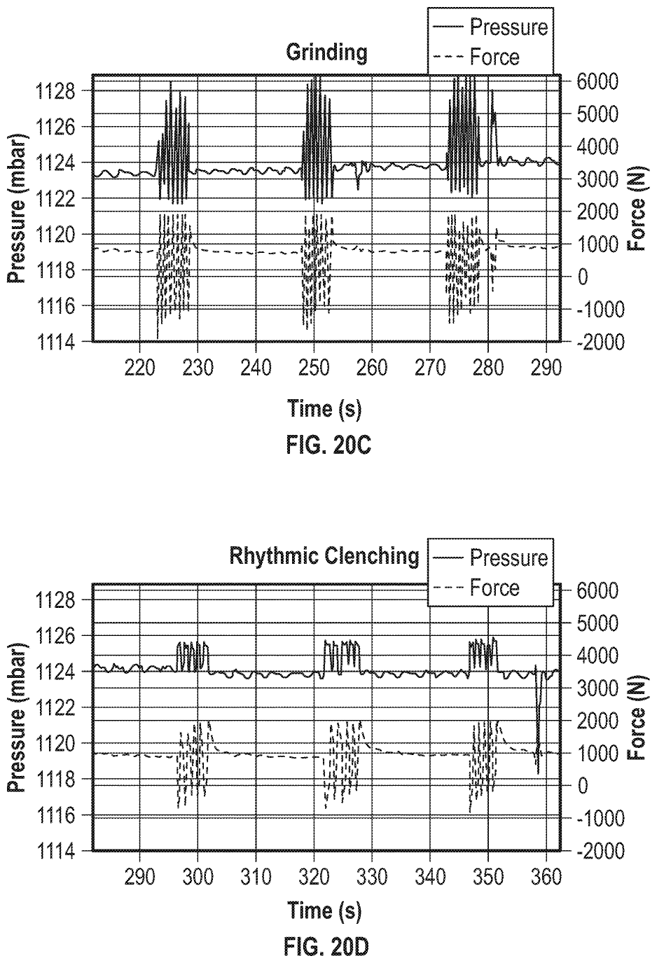

FIGS. 20A-20D are graphs illustrating example pressure and force data obtained using encapsulated pressure and force sensors, in accordance with embodiments of the present technology. In the illustrated example, an intraoral monitoring device with encapsulated pressure and force sensors was formed according to the process of FIGS. 18A-18C. The intraoral monitoring device was worn by a subject, and the subject was asked to simulate events to mimic bruxism during sleep while the intraoral monitoring device recorded pressure and force data. The simulated events include maximum voluntary contraction (MVC) (FIG. 20A), static clenching (FIG. 20B), grinding (FIG. 20C), and rhythmic clenching (FIG. 20D). As shown in FIGS. 20A-20D, each event exhibited specific pressure and force signal patterns that can be used to identify and/or predict these events. These results demonstrate that an intraoral monitoring device formed via double-layer thermoforming successfully detected MVC, clenching, and grinding.

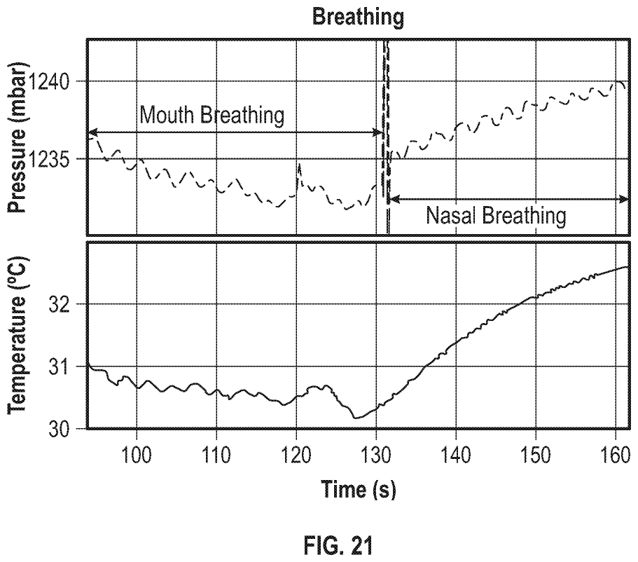

FIG. 21 shows graphs illustrating pressure data (top) and temperature data (bottom) obtained using encapsulated pressure and temperature sensors, in accordance with embodiments of the present technology. In the illustrated example, an intraoral monitoring device with encapsulated pressure and temperature sensors was formed according to the process of FIGS. 18A-18C. The intraoral monitoring device was worn by a subject, and the subject alternated between breathing through their mouth and nose. As shown in FIG. 21, during mouth breathing, temperature changes were recorded due to airflow passing through the intraoral monitoring device. During nasal breathing, the temperature rose to reach the equilibrium temperature of the oral cavity. The temperature changes induced thermal expansion/contraction of the intraoral monitoring device, deforming the air pocket trapped between the two thermoformed layers. The resulting pressure change was recorded by the pressure sensor, demonstrating a specific pattern for mouth breathing and nasal breathing.

These results indicate that the sensors encapsulated in a double-layer thermoformed air cavity can detect physiological changes that happen in the oral cavity. The force sensor within the cavity was sensitive enough to detect bruxism (e.g., clenching and grinding), and the pressure sensor in the cavity was sensitive enough to detect breathing and bruxism events. Accordingly, the double-layer thermoforming technique may provide at least the following advantages: (i) the sensors are encapsulated in a sealed air cavity, allowing the pressure sensor to detect pressure changes (e.g., deformation) occurring within the cavity due to thermal expansion/contraction (e.g., from breathing) and/or applied force (e.g., from clenching and grinding); (ii) the temperature sensor is sensitive enough to capture subtle temperature changes during breathing; and (iii) the force sensor is sensitive enough for detecting clenching and grinding.

### B. Direct Encapsulation

In some embodiments, a direct encapsulation technique includes directly potting a monitoring unit of an intraoral monitoring device in a preformed pocket using a biocompatible material (e.g., a biocompatible epoxy). In this technique, the preformed pocket is created during the fabrication of the intraoral monitoring device, and the monitoring unit is then placed into the pocket and potted with the biocompatible material. The preformed pocket can be disposed at any suitable location, such as along a lingual, buccal, and/or occlusal side of a shell of the intraoral monitoring device, e.g., on the left and/or right sides of the shell. From both bench-top and intraoral testing, sensors encapsulated within the preformed pocket using this technique are capable of sensing bruxism events (e.g., clenching and/or grinding) and breathing events (e.g., respiration patterns), among physiological parameters.

Intraoral monitoring devices with a preformed pocket can be manufactured by either a thermoforming approach or a direct fabrication (e.g., additive manufacturing) approach. For both approaches, the pocket can be designed according to the dimensions of the monitoring unit to be housed in the pocket. For instance, in embodiments where the monitoring unit 202 of FIGS. 2A-2D is used, the pocket can have a similar U-shaped geometry, e.g., the pocket can include two side portions to house the first and second end segments 214, 216, and a curved portion connecting the two side portions to house the flexible segment 218. The design of the curved portion can be adjusted based on different arches, e.g., different subjects may have different arch geometries (e.g., arch widths and/or curvatures). The customized pocket can be integrated into the intraoral monitoring device design (e.g., integrally formed with a shell of the intraoral monitoring device), e.g., placed near the lingual and palatal areas of the shell.

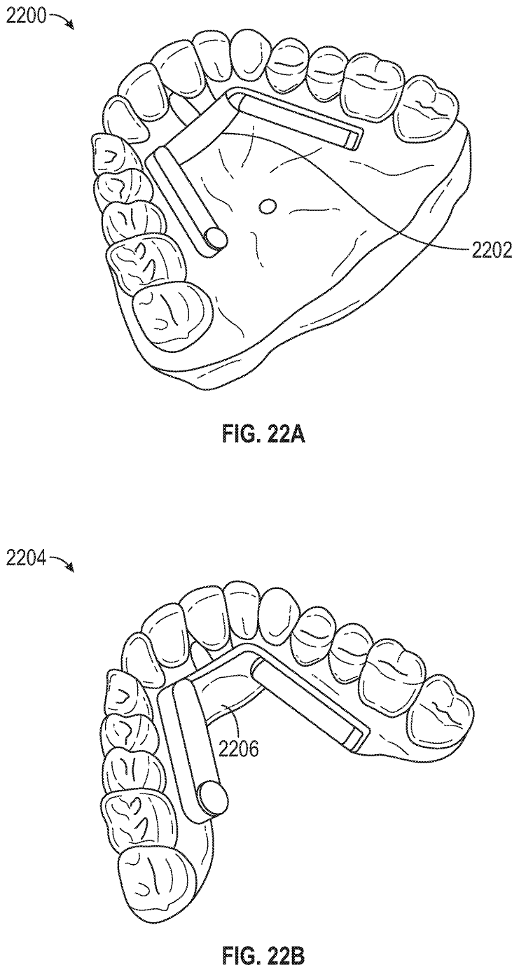

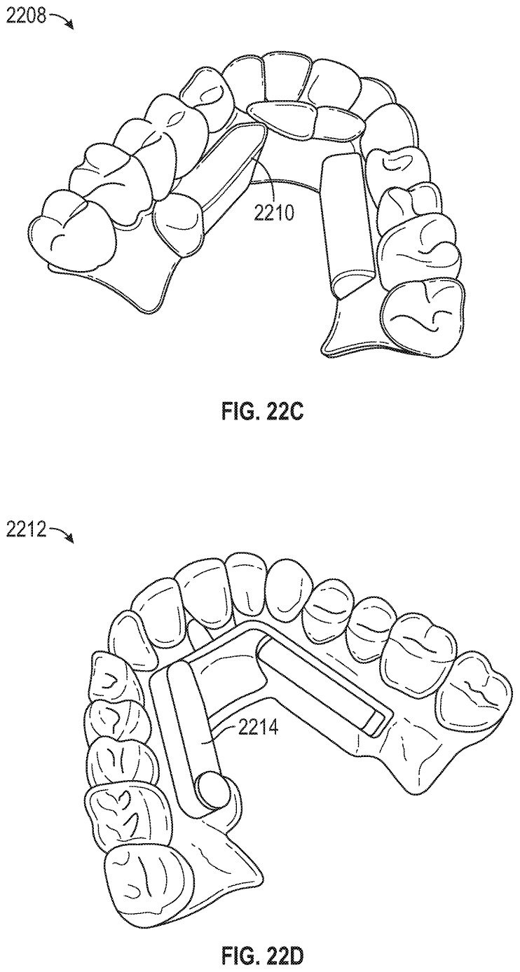

FIGS. 22A and 22B illustrate a process for forming an intraoral monitoring device with a preformed pocket via thermoforming, in accordance with embodiments of the present technology. Referring first to FIG. 22A, a mold 2200 for the intraoral monitoring device can include a portion 2202 for the pocket at the palatal area of the arch mold. Referring next to FIG. 22B, upon thermoforming over the mold 2200, the resulting intraoral monitoring device 2204 will include a preformed pocket 2206.

FIGS. 22C and 22D illustrate a process for forming an intraoral monitoring device with a preformed pocket via direct fabrication, in accordance with embodiments of the present technology. Referring first to FIG. 22C, the intraoral monitoring device can be directly fabricated (e.g., additively manufactured) based on a 3D digital model 2208, where the 3D digital model 2208 includes a preformed pocket 2210 that is directly integrated into the intraoral monitoring device design. Thus, as shown in FIG. 22D, the directly fabricated intraoral monitoring device 2212 based on the 3D digital model 2208 includes an integrally preformed pocket 2214 that is concurrently fabricated with the rest of the device 2212.

Once the customized intraoral monitoring device (e.g., a polymeric shell thereof) is fabricated, a monitoring unit including a sensor assembly and/or electronics assembly can then be placed into the preformed pocket. For instance, in embodiments where the monitoring unit 202 of FIGS. 2A-2D is used, the second end segment 216 carrying the electronics assembly 208 (e.g., including the power source 230) can be disposed in one side of the preformed pocket, and the first end segment 214 carrying the sensor assembly 206 (e.g., including the force sensor 220, pressure sensor 222, temperature sensor 224 and/or motion sensor 226) can be disposed in another side of the preformed pocket. The flexible segment 218 can be designed to sit in the curved portion of the preformed pocket and can be slightly stretched to fit into the preformed pocket. For biocompatibility and/or waterproofing of the entire intraoral monitoring device, the pocket can then be sealed. In some embodiments, the pocket can be sealed by disposing a biocompatible material (e.g., biocompatible epoxy potting) into the pocket to at least partially or fully encapsulate the sensor assembly 206 and/or the electronics assembly 208. When the pressure sensor 222 is included in the sensor assembly 206, sealing the pocket may further include building in an air pocket around at least a portion of the pressure sensor. The air pocket may deform in response to pressure changes, as described elsewhere herein.

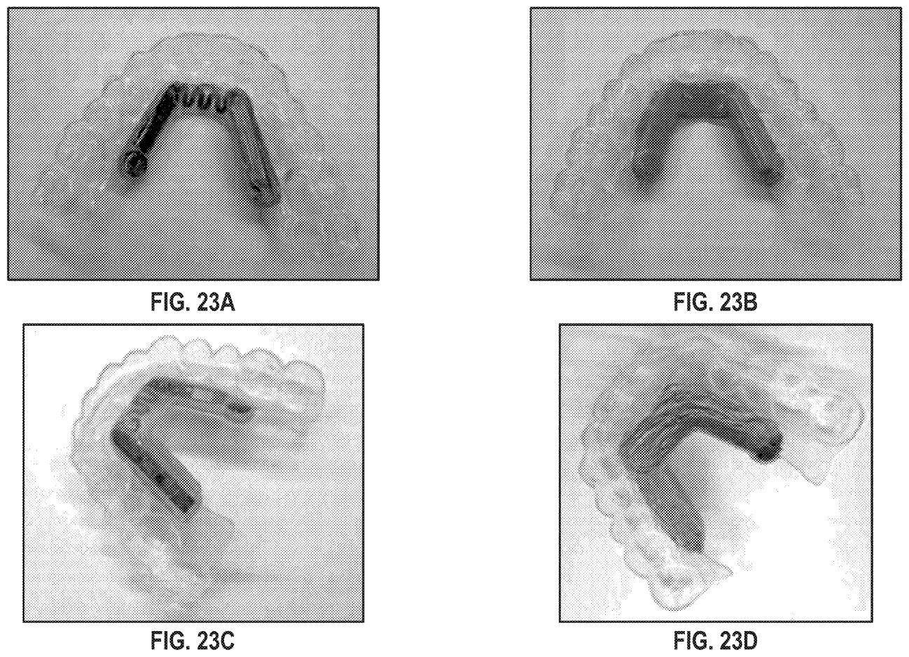

The final finish of the encapsulated pocket may be transparent (e.g., clear), translucent, opaque, and/or colored, based on the biocompatible material used. For instance, FIGS. 23A and 23B illustrate a thermoformed intraoral monitoring device with a transparent epoxy (FIG. 23A) and a colored epoxy (FIG. 23B), in accordance with embodiments of the present technology. FIGS. 23C and 23D illustrate a directly fabricated intraoral monitoring device with a transparent epoxy (FIG. 23C) and a colored epoxy (FIG. 23D), in accordance with embodiments of the present technology.

As described above, in some embodiments, the monitoring unit 1802 can be encapsulated in an appliance (e.g., an aligner, a palatal expander, a retainer) using a 3D printing method or other direct fabrication method. This approach may involve creating a digital model of the aligner with a preformed pocket designed specifically to accommodate the monitoring unit. During the 3D printing process, the aligner and the pocket may be fabricated concurrently, ensuring precise alignment and integration. Once the aligner is printed, the monitoring unit, which includes the sensor assembly and electronics, may be placed into the preformed pocket. The printing may be continued over the monitoring unit (e.g., using the same polymer material, a different polymer material, a different non-polymer material, or any combination thereof), thus encapsulating the monitoring unit therein. In some embodiments, the pocket is sealed using a separate biocompatible material (e.g., epoxy), and the printing may be continued over the sealed pocket. A direct fabrication method may allow for increased customization and more complex geometries based on individual dental anatomies, enhancing the fit and performance of the intraoral monitoring device.

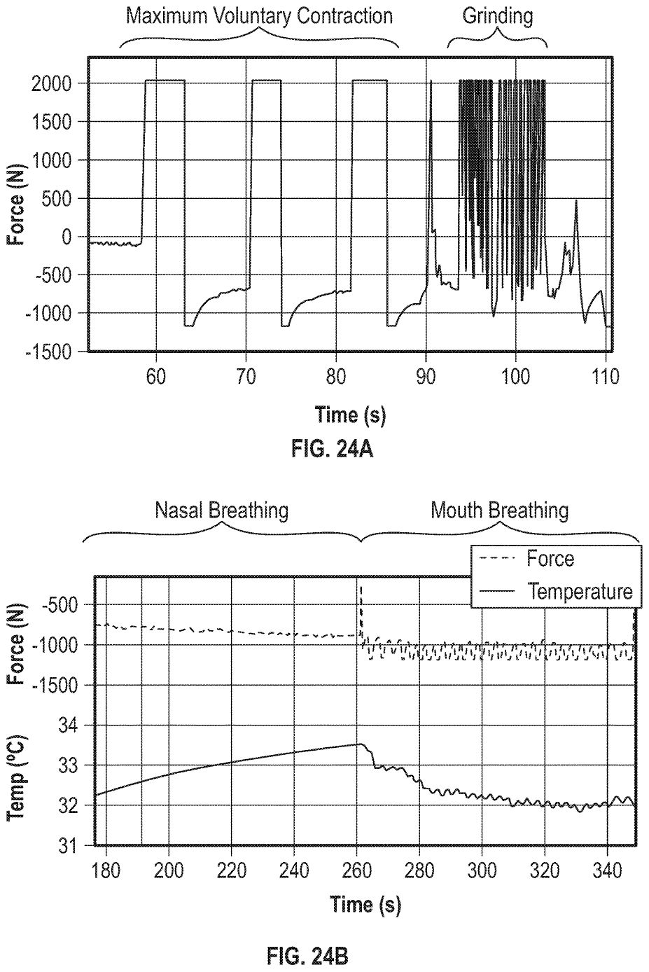

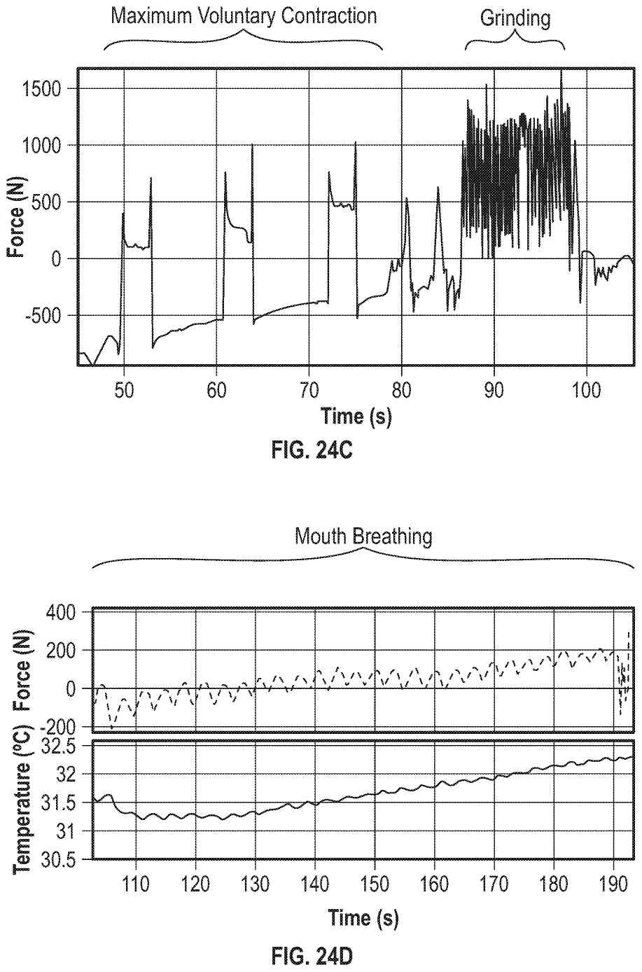

FIGS. 24A-24D are graphs illustrating example intraoral test results for an intraoral monitoring device formed using direct encapsulation processes, in accordance with embodiments of the present technology. Specifically, FIGS. 24A and 24C illustrate simulated bruxism events detected by a force sensor encapsulated in a thermoformed intraoral monitoring device (FIG. 24A) and a directly fabricated intraoral monitoring device (FIG. 24C), and FIGS. 24B and 24D illustrate breathing signals detected by a force sensor and a temperature sensor encapsulated in a thermoformed intraoral device (FIG. 24B) and a directly fabricated intraoral device (FIG. 24D). As shown in FIG. 24A, the encapsulated force sensor in the thermoformed monitoring device showed distinct responses upon simulated bruxism events (e.g., maximum voluntary clenching and grinding). As shown in FIG. 24B, the encapsulated force and temperature sensors also detected force and temperature changes during mouth breathing versus nasal breathing. Similar results were obtained using the encapsulated force and temperature sensors of the directly fabricated intraoral monitoring device (FIGS. 24C and 24D).

Alternatively or in combination, the monitoring unit can be pre-encapsulated before being positioned in the preformed pocket. For instance, the monitoring unit can be pre-encapsulated with a biocompatible material, e.g., pre-encapsulated with a biocompatible epoxy in a polydimethylsiloxane (PDMS)-based mold. After the pre-encapsulation is finished, the monitoring unit can then be directly inserted into the pocket or further bonded with the pocket using an adhesive (e.g., epoxy). This fabrication technique may provide greater feasibility for large-scale production.

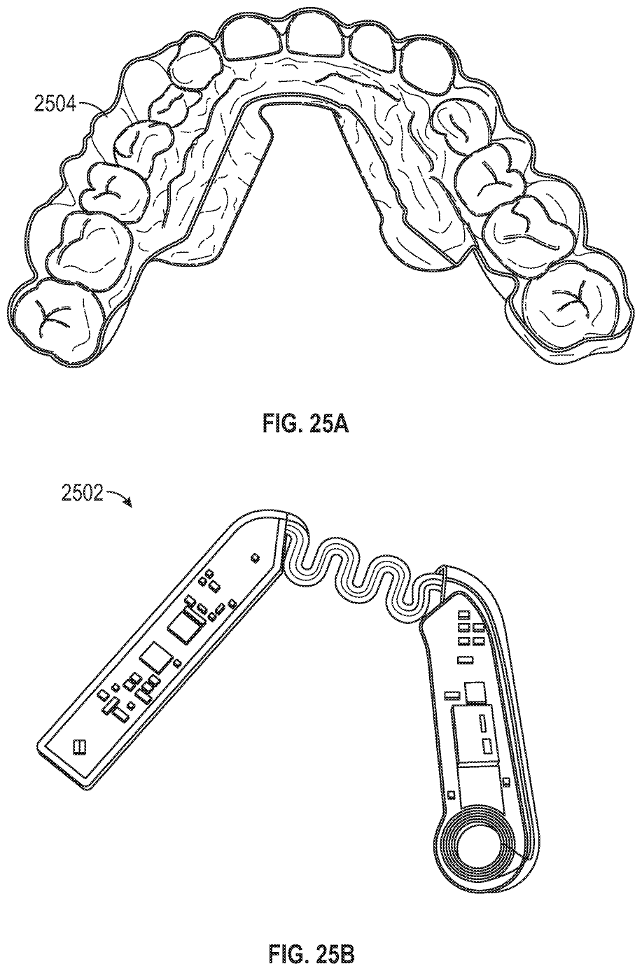

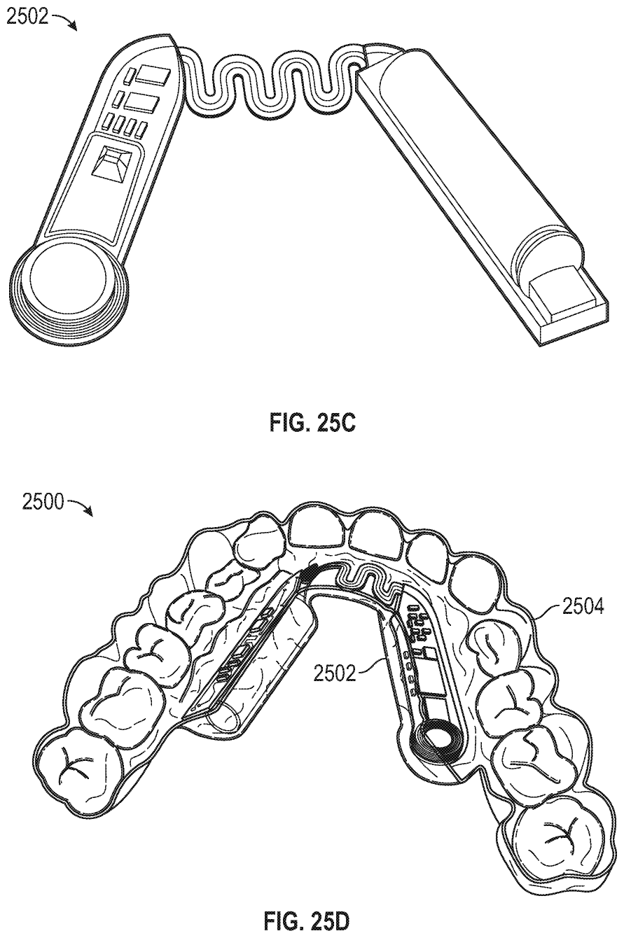

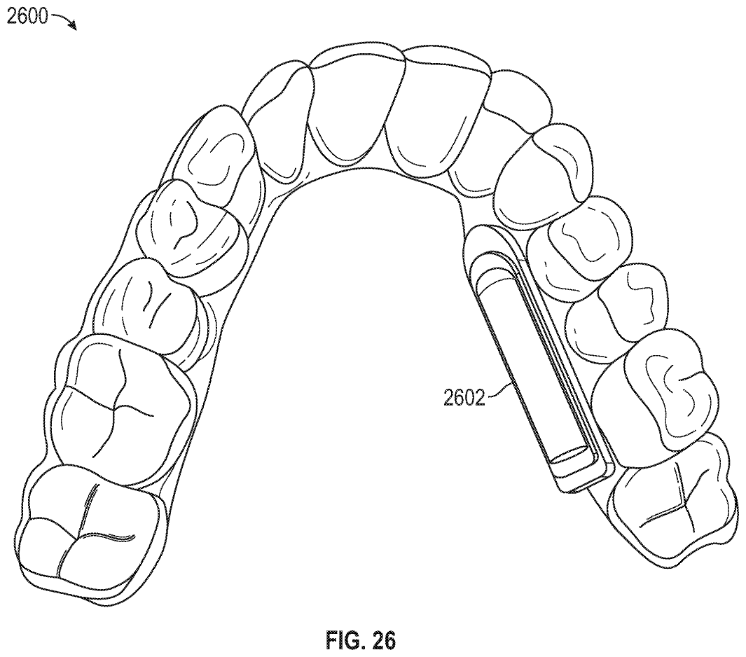

FIGS. 25A-25D illustrate an intraoral monitoring device 2500 with a pre-encapsulated monitoring unit 2502, in accordance with embodiments of the present technology. Specifically, FIG. 25A illustrates a shell 2504 of the intraoral monitoring device 2500 with a preformed pocket, FIGS. 25B and 25C are top and bottom views, respectively, of the pre-encapsulated monitoring unit 2502, and FIG. 25D illustrates the intraoral monitoring device 2500 with the pre-encapsulated monitoring unit 2502 placed in the preformed pocket.

Other configurations for an intraoral monitoring device with a directly encapsulated monitoring unit are also possible. For instance, alternative form factors for the monitoring unit can be provided, e.g., the monitoring unit may have a stacked configuration (FIG. 2E) instead of a U-shaped configuration. In such embodiments, the stacked monitoring unit may be integrated into a preformed pocket on one side of the arch of the intraoral monitoring device. FIG. 26 illustrates an intraoral monitoring device 2600 having an encapsulated monitoring unit 2602 with a stacked configuration, in accordance with embodiments of the present technology. This stacked configuration may reduce the form factor of the intraoral monitoring device 2600 and make the intraoral monitoring device 2600 more comfortable to wear during sleep. Preliminary tests showed that the intraoral monitoring device 2600 was able to detect bruxism and breathing signals.

As another example, an air pocket may be included above the pressure sensor. For instance, in some embodiments, employing epoxy potting in the encapsulation process may reduce or remove the deformable air cavity within the preformed pocket, which may make it more difficult to detect breathing patterns using a pressure sensor. To address this issue, an air pocket can be provided above the pressure sensor, e.g., using a shell formed from a soft thermoplastic material (e.g., ST30) and/or other soft encapsulation materials (e.g., silicone) that are deformable and responsive to pressure changes, thereby improving the detection of breathing patterns by the pressure sensor.

### III. Charging of Intraoral Monitoring Devices

In some embodiments, the present technology provides charging devices that may be used with any of the intraoral monitoring systems and devices described herein (e.g., any of the embodiments described in Sections I and II above). The charging devices may be suitable for use with intraoral monitoring devices having varying geometries, e.g., to accommodate subjects with different arch geometries. In some embodiments, the charging device may use wireless charging techniques. For example, the intraoral monitoring device may include charging circuitry (e.g., a first charging coil, also referred to herein as a “receiving coil”) that wirelessly receives power from the charging device (e.g., a second charging coil of the charging device, also referred to herein as a “transmitting coil”) via induction. However, it may be challenging to ensure adequate alignment of the receiving coil and transmitting coil for efficient power transfer, particularly if the geometry of the intraoral monitoring device may vary significantly from subject to subject.

The present technology can address these and other challenges by providing (1) charging devices that can properly align an intraoral monitoring device designed for any subject with the charging device (referred to herein as “designs with alignment”), or (2) charging devices that do not require alignment for efficient power transfer (referred to herein as “alignment-free designs”). The charging devices described herein can successfully overcome the alignment challenge involved in charging intraoral monitoring devices for different subjects, given the varying arch geometries of each device. For instance, for designs with alignment, the dimension of the substrate (e.g., PCB) of the monitoring unit may be used as the reference dimension for alignment, rather than the dimensions of the device shell. For the alignment-free designs, efficient charging performance may be achieved without requiring any specific alignment between the intraoral monitoring device and the transmitting coil of the charging device.

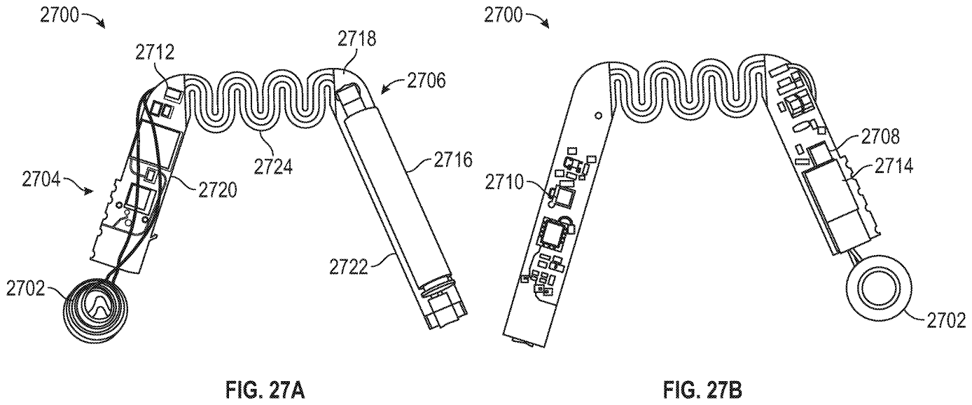

FIGS. 27A and 27B illustrate top and bottom views, respectively, of an example monitoring unit 2700 of an intraoral monitoring device having a charging coil 2702, in accordance with embodiments of the present technology. The monitoring unit 2700 may be identical or generally similar to the monitoring unit of FIGS. 2A-2D. For example, the monitoring unit 2700 can include a sensor assembly 2704 and an electronics assembly 2706. The sensor assembly 2704 can include a pressure sensor 2708 (FIG. 27B), a force sensor 2710 (FIG. 27B), and a temperature sensor 2712 (FIG. 27A). The electronics assembly 2706 can include a microprocessor 2714 (FIG. 27B) and a power source 2716 (FIG. 27A). In some embodiments, the sensor assembly 2704 and the electronics assembly 2706 are mounted on a substrate 2718. For instance, the substrate 2718 can include a first end segment 2720, a second end segment 2722, and a flexible segment 2724 between the first end segment 2720 and the second end segment 2722. The monitoring unit 2700 also includes a charging coil 2702, which may be coupled to the sensor assembly 2704 and/or electronics assembly 2706 of the monitoring unit 2700. The charging coil 2702 may be located at an end of the first end segment 2720 of the substrate 2718 (as shown), at an end of the second end segment 2722 of the substrate 2718, or at any other suitable location.

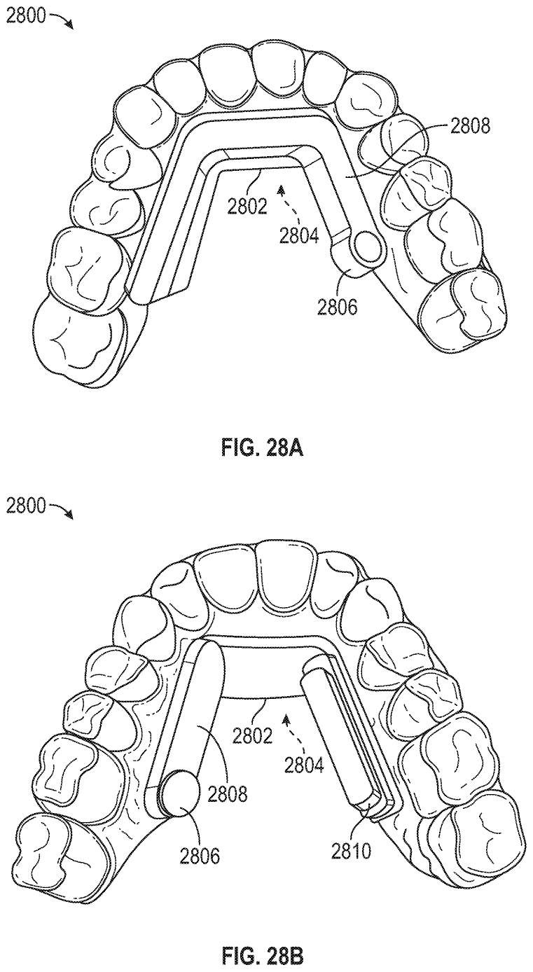

FIGS. 28A and 28B illustrate top and bottom views, respectively, of an intraoral monitoring device 2800 including a preformed pocket 2802 housing a monitoring unit 2804, in accordance with embodiments of the present technology. For example, the monitoring unit 2804 may be identical or generally similar to the monitoring unit 2700 of FIGS. 27A and 27B. The monitoring unit 2804 may be encapsulated in a biocompatible material, as previously described with respect to Section II. The preformed pocket 2802 may include portions corresponding to components of the monitoring unit 2804. For instance, the preformed pocket 2802 can include a first pocket portion 2806 corresponding to a charging coil of the monitoring unit 2804, e.g., at an end of a first end segment of the monitoring unit 2804. The preformed pocket 2802 can also include a second portion 2808 corresponding to the first end segment of the monitoring unit 2804. The preformed pocket 2802 can also include a third pocket portion 2810 corresponding to a power source of the monitoring unit 2804, e.g., at a second end segment of the monitoring unit 2804.

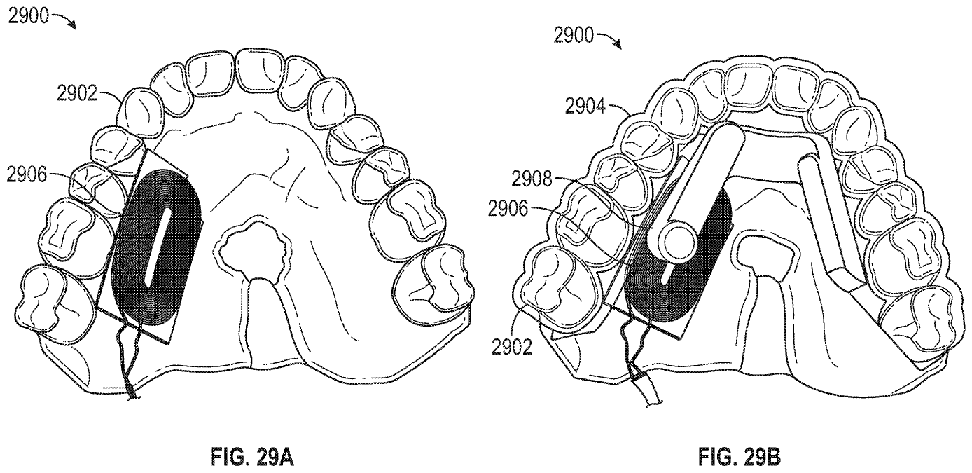

FIGS. 29A and 29B illustrate a charging device 2900 including an integrated arch mold 2902 for aligning and charging an intraoral monitoring device 2904, in accordance with embodiments of the present technology. Referring first to FIG. 29A, the charging device 2900 includes a mold 2902 of a subject's dental arch. For example, the charging device 2900 can be configured as a case (not shown) for the intraoral monitoring device 2904, and the mold 2902 can be integrated into the case. The mold 2902 can include a transmitting coil 2906 at a fixed location corresponding to the location of a receiving coil 2908 of the intraoral monitoring device 2904. Thus, as shown in FIG. 29B, when the intraoral monitoring device 2904 for the subject is placed on the mold 2902, the receiving coil 2908 of the intraoral monitoring device can be aligned with the transmitting coil 2906 coupled to the mold 2902.

Alternatively, a charging device can be designed based on dimensions of the monitoring unit of the intraoral monitoring device, such as dimensions of a substrate (e.g., PCB) of the monitoring unit. In some embodiments, a monitoring unit encapsulated in a shell (e.g., similar to the monitoring unit 202 of the intraoral monitoring device 200 of FIGS. 2A-2E), is blocked from two sides by the shell (e.g., portions of the monitoring unit 202 that abut the shell), leaving two sides exposed (e.g., portions of the monitoring unit 202 that do not abut the shell). Since the dimensions of the monitoring unit may remain fixed for all subjects, a transmitting coil of a charging device can be aligned with the receiving coil of the monitoring unit from the two exposed sides.

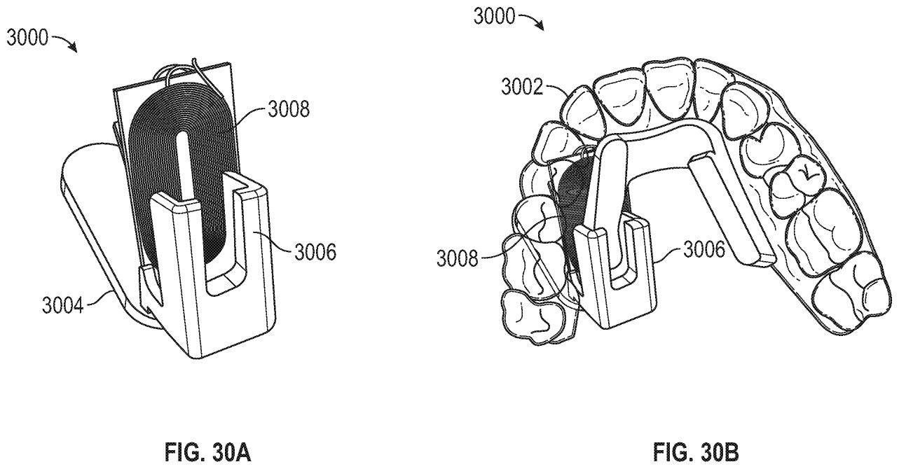

FIGS. 30A and 30B illustrate a charging device 3000 with a slide-in design for aligning and charging an intraoral monitoring device 3002, in accordance with embodiments of the present technology. The charging device 3000 can be configured as a stand including a base 3004 and a coupling mechanism 3006 for aligning the intraoral monitoring device 3002. For instance, the coupling mechanism 3006 can include a channel or similar structure having a first side that receives the monitoring unit of the intraoral monitoring device 3002, and a second side having a surface for mounting a transmitting coil 3008. The subject can slide the intraoral monitoring device 3002 into the coupling mechanism 3006, thereby ensuring alignment between the receiving coil of the intraoral monitoring device 3002 and the transmitting coil 3008 of the charging device 3000. This alignment approach can work universally for all users, regardless of the geometry of the shell of the intraoral monitoring device 3002.

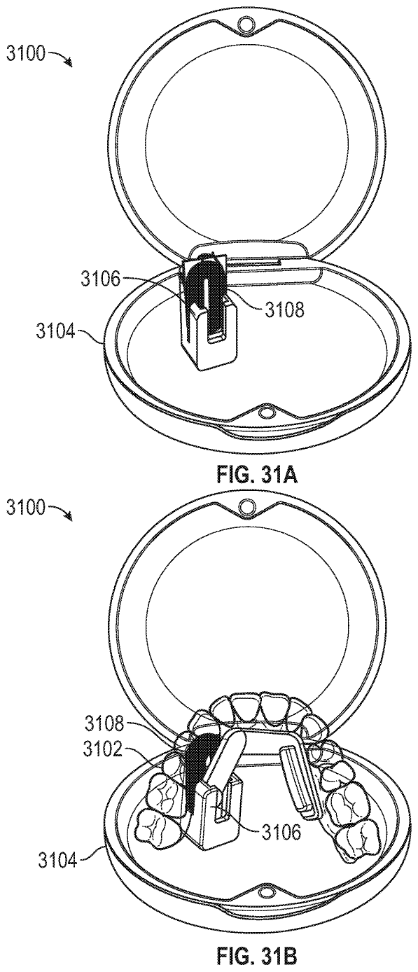

FIGS. 31A and 31B illustrate another charging device 3100 with a slide-in design for aligning and charging an intraoral monitoring device 3102, in accordance with embodiments of the present technology. The charging device 3100 can be generally similar to the charging device 3000 of FIGS. 30A and 30B, except that the charging device 3100 is configured as a case for the intraoral monitoring device 3102. For instance, the case can include a housing 3104 and a coupling mechanism 3106 coupled to the housing 3104, where the coupling mechanism 3106 includes a channel having a first side that receives the monitoring unit of the intraoral monitoring device 3102, and a second side having a surface for mounting a transmitting coil 3108. The subject can slide the intraoral monitoring device 3102 into the coupling mechanism 3106, thereby ensuring alignment between the receiving coil of the intraoral monitoring device 1002 and the transmitting coil 3108 of the charging device 3100.

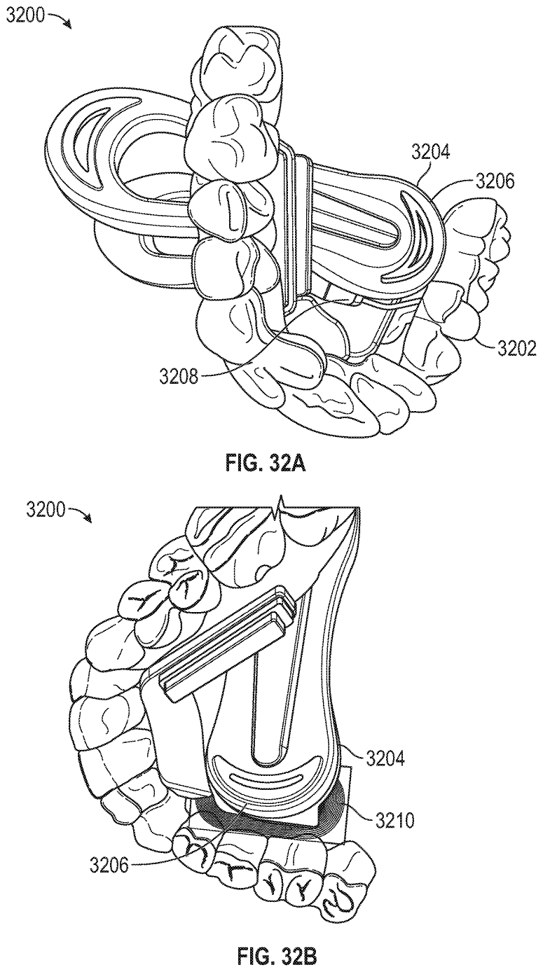

FIGS. 32A and 32B illustrate a charging device 3200 with a clipping design for aligning and charging an intraoral monitoring device 3202, in accordance with embodiments of the present technology. Similar to the slide-in design, this approach can be based on the dimensions of the monitoring unit of the intraoral monitoring device 3202, such as the dimensions of a substrate of the monitoring unit. The charging device 3200 can include a coupling mechanism 3204 for aligning the intraoral monitoring device 3202. In some embodiments, the coupling mechanism 3204 is configured as a clip for securing at least a portion of the intraoral monitoring device 3202. The clip can include a first end 3206 having a pocket that accommodates the monitoring unit of the intraoral monitoring device 3202, and a second end 3208 that carries a transmitting coil 3210. Thus, by clipping the coupling mechanism 3204 over the monitoring unit, alignment of the receiving coil of the intraoral monitoring device 3202 with the transmitting coil 3210 can be achieved. The charging device 3200 can be configured as a standalone charging device (as shown) or can be configured as a case for the intraoral monitoring device 3202 (e.g., similar to the charging device 3100 of FIGS. 31A and 31B).

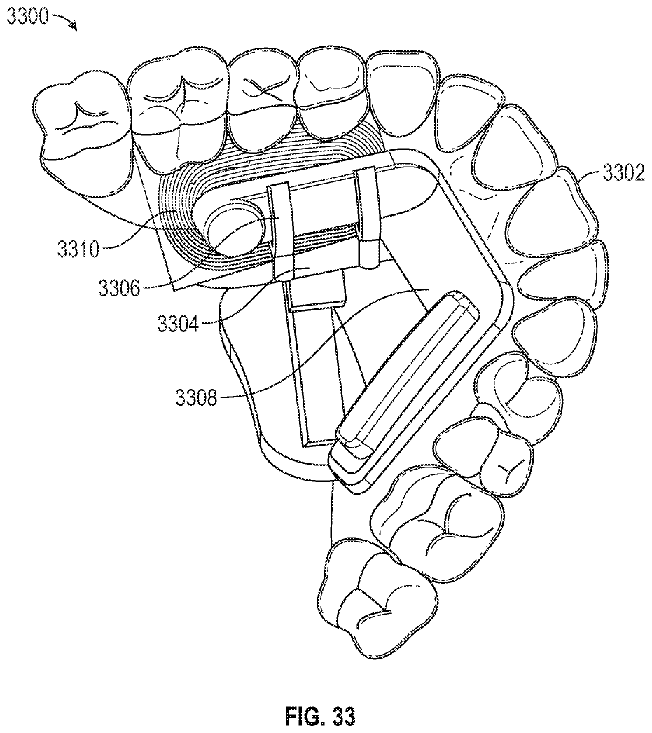

FIG. 33 illustrates a charging device 3300 with a hook design for aligning and charging an intraoral monitoring device 3302, in accordance with embodiments of the present technology. The charging device 3300 can include a coupling mechanism 3304. In some embodiments, the coupling mechanism 3304 is configured as one or more hooks 3306 for supporting at least a portion of the intraoral monitoring device 3302. For example, the coupling mechanism 3304 can include two hooks 3306 that hold a monitoring unit 3308 of the intraoral monitoring device 3302. The dimension of each hook 3306 and/or the distance between the hooks 3306 can be configured based on the geometry of the monitoring unit, so that the hooks 3306 can hold the intraoral monitoring device 3302 in place while aligning the receiving coil of the intraoral monitoring device 3302 with a transmitting coil 3310 for efficient charging. In some embodiments, the hook design shown in FIG. 33 may be easier to clean than other designs, since the hooks 3306 are open structures that may be less susceptible to pathogen and/or dirt accumulation, and/or can be cleaned more easily.

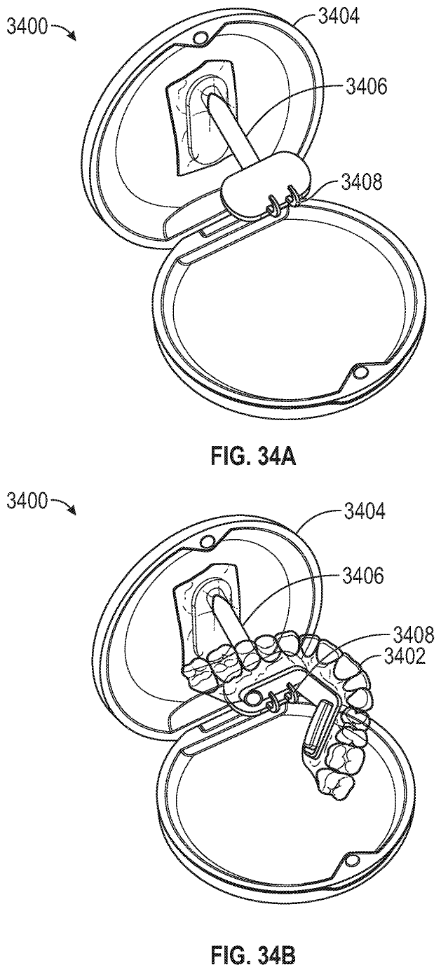

FIGS. 34A and 34B illustrate another charging device 3400 with a hook design for aligning and charging an intraoral monitoring device 3402, in accordance with embodiments of the present technology. The charging device 3400 can be generally similar to the charging device 3300 of FIG. 33, except that the charging device 3400 is configured as a case for the intraoral monitoring device 3402. For instance, the case can include a housing 3404 and a coupling mechanism 3406 coupled to the housing 3404, where the coupling mechanism 3406 includes one or more hooks 3408 for supporting at least a portion of the intraoral monitoring device 3402, and for aligning the transmitting coil of the charging device 3400 to the receiving coil of the intraoral monitoring device 3402. Optionally, the coupling mechanism 3406 can be foldable, e.g., the coupling mechanism 3406 can be folded into the case to keep it more compact for storage when not in use, and can be unfolded out to support the intraoral monitoring device 3402 for charging while the case is open.

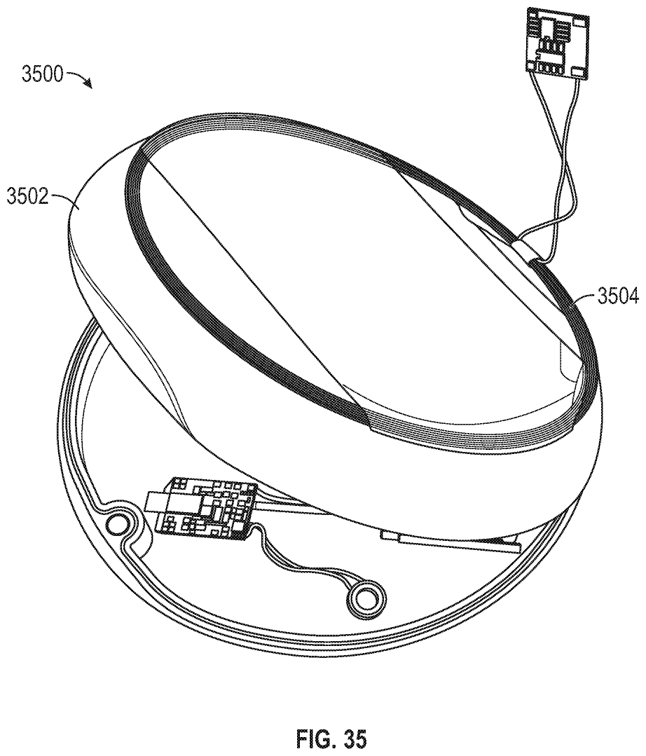

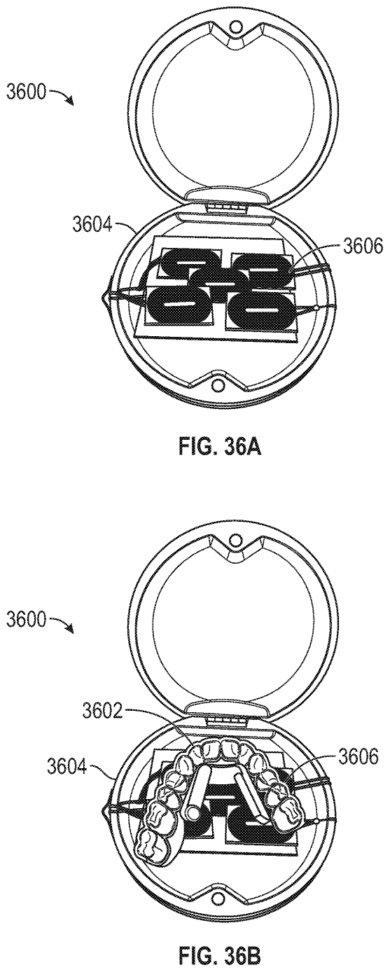

FIG. 35 illustrates a charging device 3500 with an alignment-free design for charging an intraoral monitoring device, in accordance with embodiments of the present technology. The charging device 3500 can be configured as a case for the intraoral monitoring device. The case can include a housing 3502 with a single large transmitting coil 3504 to make the entire interior of the case an active charging region. Accordingly, the intraoral monitoring device can be charged when placed anywhere within the case.

FIGS. 36A and 36B illustrate another charging device 3600 with an alignment-free design for charging an intraoral monitoring device 3602, in accordance with embodiments of the present technology. The charging device 3600 can be configured as a case for the intraoral monitoring device. The case includes a housing 3604 with a plurality of smaller transmitting coils 3606, as opposed to a single large transmitting coil. Optionally, the transmitting coils 3606 may be selectively activated. For instance, feedback from the transmitting coils 3606 may be detected and used to identify which transmitting coil(s) 3606 have the closest proximity and/or proper orientation for coupling with the receiving coil of the intraoral monitoring device 3602. Then, the identified transmitting coils 3606 may be selectively activated, thereby achieving proper charging without excess power transmission.

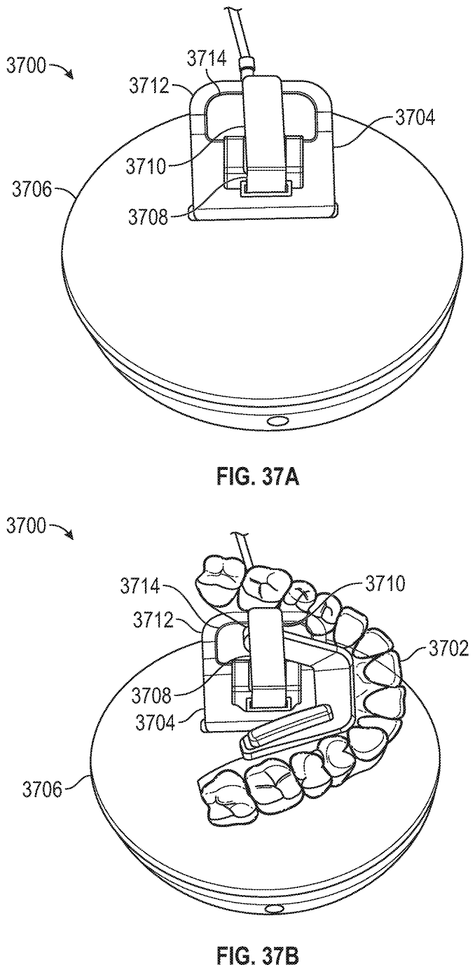

FIGS. 37A and 37B illustrate another charging device 3700 with a clipping design for aligning and charging an intraoral monitoring device 3702, in accordance with embodiments of the present technology. The charging device 3700 can be generally similar to the charging device 3200 of FIGS. 32A and 32B, except that the charging device 3700 is configured as a stand 3704 mounted on an open platform 3706. The stand 3704 can include a coupling mechanism 3708 for aligning the intraoral monitoring device 3702. For example, the coupling mechanism 3708 can include a clip 3710 for securing at least a portion of the intraoral monitoring device 3702 to the stand 3704, where the clip includes a pocket that accommodates the monitoring unit of the intraoral monitoring device. The stand 3704 can include a plate 3712 opposing the clip 3710, with a transmitting coil 3714 being located within the plate 3712. Accordingly, when the intraoral monitoring device 3702 is clipped against the plate 3712, the receiving coil of the intraoral monitoring device 3702 can be aligned with the transmitting coil 3714.

### IV. Dental Appliances and Associated Methods

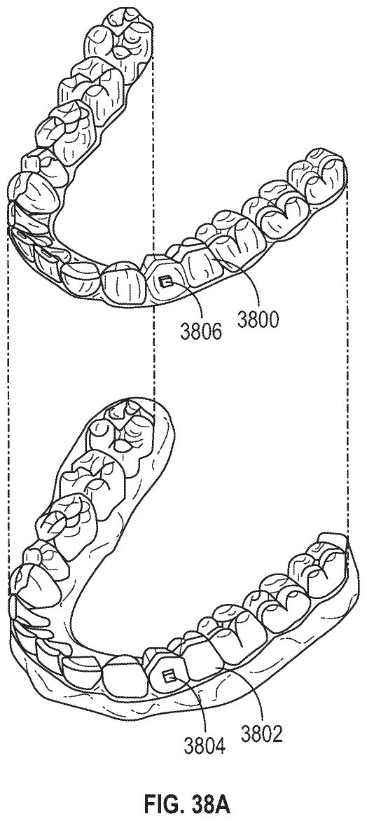

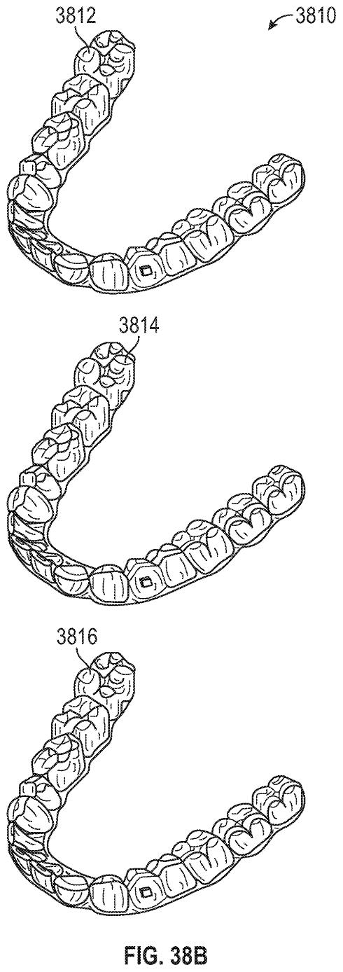

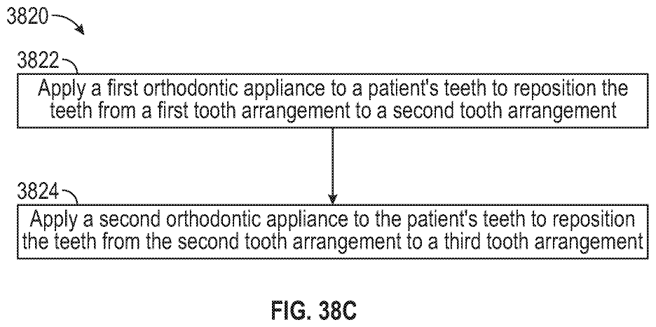

FIG. 38A illustrates a representative example of a tooth repositioning appliance 3800 configured in accordance with embodiments of the present technology. The appliance 3800 can be used with any of the systems, methods, and devices described herein (e.g., in Sections I-III above). The appliance 3800 (also referred to herein as an “aligner”) can be worn by a patient in order to achieve an incremental repositioning of individual teeth 3802 in the jaw. The appliance 3800 can include a shell (e.g., a continuous polymeric shell or a segmented shell) having teeth-receiving cavities that receive and resiliently reposition the teeth. The appliance 3800 or portion(s) thereof may be indirectly fabricated using a physical model of teeth. For example, an appliance (e.g., polymeric appliance) can be formed using a physical model of teeth and a sheet of suitable layers of polymeric material. In some embodiments, a physical appliance is directly fabricated, e.g., using additive manufacturing techniques, from a digital model of an appliance.

The appliance 3800 can fit over all teeth present in an upper or lower jaw, or less than all of the teeth. The appliance 3800 can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance 3800 can be a generic appliance configured to receive the teeth, but not necessarily shaped to match the topography of the patient's teeth. In some cases, only certain teeth received by the appliance 3800 are repositioned by the appliance 3800 while other teeth can provide a base or anchor region for holding the appliance 3800 in place as it applies force against the tooth or teeth targeted for repositioning. In some cases, some, most, or even all of the teeth can be repositioned at some point during treatment. Teeth that are moved can also serve as a base or anchor for holding the appliance as it is worn by the patient. In preferred embodiments, no wires or other means are provided for holding the appliance 3800 in place over the teeth. In some cases, however, it may be desirable or necessary to provide individual attachments 3804 or other anchoring elements on teeth 3802 with corresponding receptacles 3806 or apertures in the appliance 3800 so that the appliance 3800 can apply a selected force on the tooth. Representative examples of appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the url “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

FIG. 38B illustrates a tooth repositioning system 3810 including a plurality of appliances 3812, 3814, 3816, in accordance with embodiments of the present technology. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 3810 can include a first appliance 3812 corresponding to an initial tooth arrangement, one or more intermediate appliances 3814 corresponding to one or more intermediate arrangements, and a final appliance 3816 corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

FIG. 38C illustrates a method 3820 of orthodontic treatment using a plurality of appliances, in accordance with embodiments of the present technology. The method 3820 can be practiced using any of the appliances or appliance sets described herein. In block 3822, a first orthodontic appliance is applied to a patient's teeth in order to reposition the teeth from a first tooth arrangement to a second tooth arrangement. In block 3824, a second orthodontic appliance is applied to the patient's teeth in order to reposition the teeth from the second tooth arrangement to a third tooth arrangement. The method 3820 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

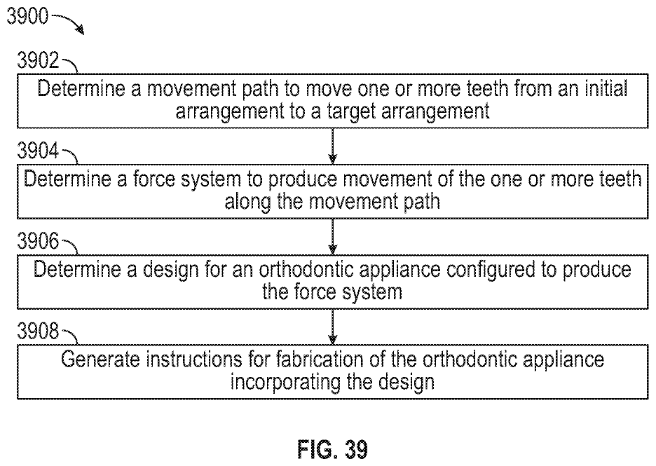

FIG. 39 illustrates a method 3900 for designing an orthodontic appliance, in accordance with embodiments of the present technology. The method 3900 can be applied to any embodiment of the orthodontic appliances described herein. Some or all of the steps of the method 3900 can be performed by any suitable data processing system or device, e.g., one or more processors configured with suitable instructions.

In block 3902, a movement path to move one or more teeth from an initial arrangement to a target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. From the obtained data, a digital data set can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, including measured or extrapolated hidden surfaces and root structures, as well as surrounding bone and soft tissue.

The target arrangement of the teeth (e.g., a desired and intended end result of orthodontic treatment) can be received from a clinician in the form of a prescription, can be calculated from basic orthodontic principles, and/or can be extrapolated computationally from a clinical prescription. With a specification of the desired final positions of the teeth and a digital representation of the teeth themselves, the final position and surface geometry of each tooth can be specified to form a complete model of the tooth arrangement at the desired end of treatment.

Having both an initial position and a target position for each tooth, a movement path can be defined for the motion of each tooth. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In block 3904, a force system to produce movement of the one or more teeth along the movement path is determined. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

Determination of the force system can be performed in a variety of ways. For example, in some embodiments, the force system is determined on a patient-by-patient basis, e.g., using patient-specific data. Alternatively or in combination, the force system can be determined based on a generalized model of tooth movement (e.g., based on experimentation, modeling, clinical data, etc.), such that patient-specific data is not necessarily used. In some embodiments, determination of a force system involves calculating specific force values to be applied to one or more teeth to produce a particular movement. Alternatively, determination of a force system can be performed at a high level without calculating specific force values for the teeth. For instance, block 3904 can involve determining a particular type of force to be applied (e.g., extrusive force, intrusive force, translational force, rotational force, tipping force, torquing force, etc.) without calculating the specific magnitude and/or direction of the force.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully formed suture. Thus, in juvenile patients and others without fully closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as X-ray data or 3D optical scanning data, for example, can be used to determine parameters of the skeletal and muscular system of the patient's mouth, so as to determine forces sufficient to provide a desired expansion of the palate and/or arch. In some embodiments, the thickness and/or density of the mid-palatal suture may be measured, or input by a treating professional. In other embodiments, the treating professional can select an appropriate treatment based on physiological characteristics of the patient. For example, the properties of the palate may also be estimated based on factors such as the patient's age—for example, young juvenile patients can require lower forces to expand the suture than older patients, as the suture has not yet fully formed.

In block 3906, a design for an orthodontic appliance configured to produce the force system is determined. The design can include the appliance geometry, material composition and/or material properties, and can be determined in various ways, such as using a treatment or force application simulation environment. A simulation environment can include, e.g., computer modeling systems, biomechanical systems or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from a number of vendors can be used, including finite element analysis packages from ANSYS, Inc., of Canonsburg, PA, and SIMULIA (Abaqus) software products from Dassault Systèmes of Waltham, MA.

Optionally, one or more designs can be selected for testing or force modeling. As noted above, a desired tooth movement, as well as a force system required or desired for eliciting the desired tooth movement, can be identified. Using the simulation environment, a candidate design can be analyzed or modeled for determination of an actual force system resulting from use of the candidate appliance. One or more modifications can optionally be made to a candidate appliance, and force modeling can be further analyzed as described, e.g., in order to iteratively determine an appliance design that produces the desired force system.

In block 3908, instructions for fabrication of the orthodontic appliance incorporating the design are generated. The instructions can be configured to control a fabrication system or device in order to produce the orthodontic appliance with the specified design. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by thermoforming.

Although the above steps show a method 3900 of designing an orthodontic appliance in accordance with some embodiments, a person of ordinary skill in the art will recognize some variations based on the teaching described herein. Some of the steps may comprise sub-steps. Some of the steps may be repeated as often as desired. One or more steps of the method 3900 may be performed with any suitable fabrication system or device, such as the embodiments described herein. Some of the steps may be optional, e.g., the process of block 3904 can be omitted, such that the orthodontic appliance is designed based on the desired tooth movements and/or determined tooth movement path, rather than based on a force system. Moreover, the order of the steps can be varied as desired.

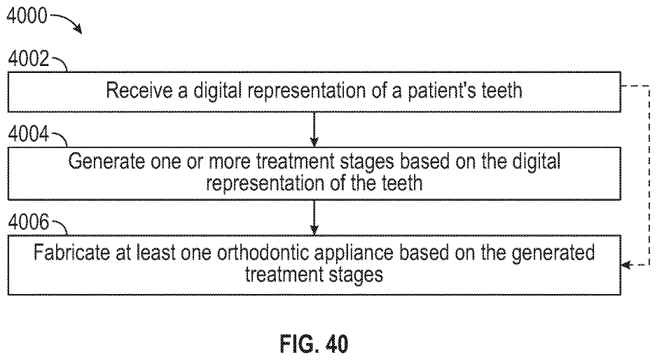

FIG. 40 illustrates a method 4000 for digitally planning an orthodontic treatment and/or design or fabrication of an appliance, in accordance with embodiments. The method 4000 can be applied to any of the treatment procedures described herein and can be performed by any suitable data processing system.

In block 4002, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In block 4004, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In block 4006, at least one orthodontic appliance is fabricated based on the generated treatment stages. For example, a set of appliances can be fabricated, each shaped according to a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired.

In some instances, staging of various arrangements or treatment stages may not be necessary for design and/or fabrication of an appliance. As illustrated by the dashed line in FIG. 40, design and/or fabrication of an orthodontic appliance, and perhaps a particular orthodontic treatment, may include use of a representation of the patient's teeth (e.g., including receiving a digital representation of the patient's teeth (block 4002)), followed by design and/or fabrication of an orthodontic appliance based on a representation of the patient's teeth in the arrangement represented by the received representation.

As noted herein, the techniques described herein can be used with directly fabricated dental appliances, such as aligners and/or a series of aligners with tooth-receiving cavities configured to move a person's teeth from an initial arrangement toward a target arrangement in accordance with a treatment plan. Aligners can include mandibular repositioning elements, such as those described in U.S. Pat. No. 10,912,629, entitled “Dental Appliances with Repositioning Jaw Elements,” filed Nov. 30, 2015; U.S. Pat. No. 10,537,406, entitled “Dental Appliances with Repositioning Jaw Elements,” filed Sep. 19, 2014; and U.S. Pat. No. 9,844,424, entitled “Dental Appliances with Repositioning Jaw Elements,” filed Feb. 21, 2014; all of which are incorporated by reference herein in their entirety.

The techniques described herein can also be used with attachment placement devices, e.g., appliances used to position prefabricated attachments on a person's teeth in accordance with one or more aspects of a treatment plan. Examples of attachment placement devices (also known as “attachment placement templates” or “attachment fabrication templates”) can be found at least in: U.S. application Ser. No. 17/249,218, entitled “Flexible 3D Printed Orthodontic Device,” filed Feb. 24, 2021; U.S. application Ser. No. 16/366,686, entitled “Dental Attachment Placement Structure,” filed Mar. 27, 2019; U.S. application Ser. No. 15/674,662, entitled “Devices and Systems for Creation of Attachments,” filed Aug. 11, 2017; U.S. Pat. No. 11,103,330, entitled “Dental Attachment Placement Structure,” filed Jun. 14, 2017; U.S. application Ser. No. 14/963,527, entitled “Dental Attachment Placement Structure,” filed Dec. 9, 2015; U.S. application Ser. No. 14/939,246, entitled “Dental Attachment Placement Structure,” filed Nov. 12, 2015; U.S. application Ser. No. 14/939,252, entitled “Dental Attachment Formation Structures,” filed Nov. 12, 2015; and U.S. Pat. No. 9,700,385, entitled “Attachment Structure,” filed Aug. 22, 2014; all of which are incorporated by reference herein in their entirety.

The techniques described herein can be used with incremental palatal expanders and/or a series of incremental palatal expanders used to expand a person's palate from an initial position toward a target position in accordance with one or more aspects of a treatment plan. Examples of incremental palatal expanders can be found at least in: U.S. application Ser. No. 16/380,801, entitled “Releasable Palatal Expanders,” filed Apr. 10, 2019; U.S. application Ser. No. 16/022,552, entitled “Devices, Systems, and Methods for Dental Arch Expansion,” filed Jun. 28, 2018; U.S. Pat. No. 11,045,283, entitled “Palatal Expander with Skeletal Anchorage Devices,” filed Jun. 8, 2018; U.S. application Ser. No. 15/831,159, entitled “Palatal Expanders and Methods of Expanding a Palate,” filed Dec. 4, 2017; U.S. Pat. No. 10,993,783, entitled “Methods and Apparatuses for Customizing a Rapid Palatal Expander,” filed Dec. 4, 2017; and U.S. Pat. No. 7,192,273, entitled “System and Method for Palatal Expansion,” filed Aug. 7, 2003; all of which are incorporated by reference herein in their entirety.

### V. Overview of Additive Manufacturing Technology

The systems, methods, and devices described herein (e.g., in Sections I-IV above) may be fabricated using a wide variety of additive manufacturing techniques. Examples of additive manufacturing techniques include, but are not limited to, the following: (1) vat photopolymerization, in which an object is constructed from a vat or other bulk source of liquid photopolymer resin, including techniques such as stereolithography (SLA), digital light processing (DLP), continuous liquid interface production (CLIP), two-photon induced photopolymerization (TPIP), and volumetric additive manufacturing; (2) material jetting, in which material is jetted onto a build platform, such as using either a continuous or drop on demand (DOD) approach; (3) binder jetting, in which alternating layers of a build material (e.g., a powder-based material) and a binding material (e.g., a liquid binder) are deposited by a print head; (4) material extrusion, in which material is drawn though a nozzle, heated, and deposited layer-by-layer, such as fused deposition modeling (FDM) and direct ink writing (DIW); (5) powder bed fusion, including techniques such as direct metal laser sintering (DMLS), electron beam melting (EBM), selective heat sintering (SHS), selective laser melting (SLM), and selective laser sintering (SLS); (6) sheet lamination, including techniques such as laminated object manufacturing (LOM) and ultrasonic additive manufacturing (UAM); and (7) directed energy deposition, including techniques such as laser engineering net shaping, directed light fabrication, direct metal deposition, and 3D laser cladding. Optionally, an additive manufacturing process can use a combination of two or more additive manufacturing techniques.

For example, the additively manufactured object can be fabricated using a vat photopolymerization process in which light is used to selectively cure a vat or other bulk source of a curable material (e.g., a polymeric resin). Each layer of curable material can be selectively exposed to light in a single exposure (e.g., DLP) or by scanning a beam of light across the layer (e.g., SLA). Vat polymerization can be performed in a “top-down” or “bottom-up” approach, depending on the relative locations of the material source, light source, and build platform.

As another example, the additively manufactured object can be fabricated using high temperature lithography (also known as “hot lithography”). High temperature lithography can include any photopolymerization process that involves heating a photopolymerizable material (e.g., a polymeric resin). For example, high temperature lithography can involve heating the material to a temperature of at least 30° C., 40° C., 50° C., 60° C., 70° C., 80° C., 90° C., 100° C., 110° C., or 120° C. In some embodiments, the material is heated to a temperature within a range from 50° C. to 120° C., from 90° C. to 120° C., from 100° C. to 120° C., from 105° C. to 115° C., or from 105° C. to 110° C. The heating can lower the viscosity of the photopolymerizable material before and/or during curing, and/or increase reactivity of the photopolymerizable material. Accordingly, high temperature lithography can be used to fabricate objects from highly viscous and/or poorly flowable materials, which, when cured, may exhibit improved mechanical properties (e.g., stiffness, strength, stability) compared to other types of materials. For example, high temperature lithography can be used to fabricate objects from a material having a viscosity of at least 5 Pa-s, 10 Pa-s, 15 Pa-s, 20 Pa-s, 30 Pa-s, 40 Pa-s, or 50 Pa-s at 20° C. Representative examples of high-temperature lithography processes that may be incorporated in the methods herein are described in International Publication Nos. WO 2015/075094, WO 2016/078838, WO 2018/032022, WO 2020/070639, WO 2021/130657, and WO 2021/130661, the disclosures of each of which are incorporated herein by reference in their entirety.

In some embodiments, the additively manufactured object is fabricated using continuous liquid interphase production (also known as “continuous liquid interphase printing”) in which the object is continuously built up from a reservoir of photopolymerizable resin by forming a gradient of partially cured resin between the building surface of the object and a polymerization-inhibited “dead zone.” In some embodiments, a semi-permeable membrane is used to control transport of a photopolymerization inhibitor (e.g., oxygen) into the dead zone in order to form the polymerization gradient. Representative examples of continuous liquid interphase production processes that may be incorporated in the methods herein are described in U.S. Patent Publication Nos. 2015/0097315, 2015/0097316, and 2015/0102532, the disclosures of each of which are incorporated herein by reference in their entirety.

As another example, a continuous additive manufacturing method can achieve continuous build-up of an object geometry by continuous movement of the build platform (e.g., along the vertical or Z-direction) during the irradiation phase, such that the hardening depth of the irradiated photopolymer is controlled by the movement speed. Accordingly, continuous polymerization of material on the build surface can be achieved. Such methods are described in U.S. Pat. No. 7,892,474, the disclosure of which is incorporated herein by reference in its entirety. In another example, a continuous additive manufacturing method can involve extruding a composite material composed of a curable liquid material surrounding a solid strand. The composite material can be extruded along a continuous three-dimensional path in order to form the object. Such methods are described in U.S. Pat. No. 10,162,264 and U.S. Patent Publication No. 2014/0061974, the disclosures of which are incorporated herein by reference in their entirety. In yet another example, a continuous additive manufacturing method can utilize a “heliolithography” approach in which the liquid photopolymer is cured with focused radiation while the build platform is continuously rotated and raised. Accordingly, the object geometry can be continuously built up along a spiral build path. Such methods are described in U.S. Patent Publication No. 2014/0265034, the disclosure of which is incorporated herein by reference in its entirety.

In a further example, the additively manufactured object can be fabricated using a volumetric additive manufacturing (VAM) process in which an entire object is produced from a 3D volume of resin in a single print step, without requiring layer-by-layer build up. During a VAM process, the entire build volume is irradiated with energy, but the projection patterns are configured such that only certain voxels will accumulate a sufficient energy dosage to be cured. Representative examples of VAM processes that may be incorporated into the present technology include tomographic volumetric printing, holographic volumetric printing, multiphoton volumetric printing, and xolography. For instance, a tomographic VAM process can be performed by projecting 2D optical patterns into a rotating volume of photosensitive material at perpendicular and/or angular incidences to produce a cured 3D structure. A holographic VAM process can be performed by projecting holographic light patterns into a stationary reservoir of photosensitive material. A xolography process can use photoswitchable photoinitiators to induce local polymerization inside a volume of photosensitive material upon linear excitation by intersecting light beams of different wavelengths. Additional details of VAM processes suitable for use with the present technology are described in U.S. Pat. No. 11,370,173, U.S. Patent Publication No. 2021/0146619, U.S. Patent Publication No. 2022/0227051, International Publication No. WO 2017/115076, International Publication No. WO 2020/245456, International Publication No. WO 2022/011456, and U.S. Provisional Patent Application No. 63/181,645, the disclosures of each of which are incorporated herein by reference in their entirety.

In yet another example, the additively manufactured object can be fabricated using a powder bed fusion process (e.g., selective laser sintering) involving using a laser beam to selectively fuse a layer of powdered material according to a desired cross-sectional shape in order to build up the object geometry. As another example, the additively manufactured object can be fabricated using a material extrusion process (e.g., fused deposition modeling) involving selectively depositing a thin filament of material (e.g., thermoplastic polymer) in a layer-by-layer manner in order to form an object. In yet another example, the additively manufactured object can be fabricated using a material jetting process involving jetting or extruding one or more materials onto a build surface in order to form successive layers of the object geometry (e.g., inkjet printing).

The additively manufactured object can be made of any suitable material or combination of materials. As discussed above, in some embodiments, the additively manufactured object is made partially or entirely out of a polymeric material, such as a curable polymeric resin. The resin can be composed of one or more monomer components that are initially in a liquid state. The resin can be in the liquid state at room temperature (e.g., 20° C.) or at an elevated temperature (e.g., a temperature within a range from 50° C. to 120° C.). When exposed to energy (e.g., light), the monomer components can undergo a polymerization reaction such that the resin solidifies into the desired object geometry. Representative examples of curable polymeric resins and other materials suitable for use with the additive manufacturing techniques herein are described in International Publication Nos. WO 2019/006409, WO 2020/070639, and WO 2021/087061, the disclosures of each of which are incorporated herein by reference in their entirety.

Optionally, the additively manufactured object can be fabricated from a plurality of different materials (e.g., at least two, three, four, five, or more different materials). The materials can differ from each other with respect to composition, curing conditions (e.g., curing energy wavelength), material properties before curing (e.g., viscosity), material properties after curing (e.g., stiffness, strength, transparency), and so on. In some embodiments, the additively manufactured object is formed from multiple materials in a single manufacturing step. For instance, a multi-tip extrusion apparatus can be used to selectively dispense multiple types of materials from distinct material supply sources in order to fabricate an object from a plurality of different materials. Examples of such methods are described in U.S. Pat. Nos. 6,749,414 and 11,318,667, the disclosures of which are incorporated herein by reference in their entirety. Alternatively or in combination, the additively manufactured object can be formed from multiple materials in a plurality of sequential manufacturing steps. For instance, a first portion of the object can be formed from a first material in accordance with any of the fabrication methods herein, then a second portion of the object can be formed from a second material in accordance with any of the fabrication methods herein, and so on, until the entirety of the object has been formed.

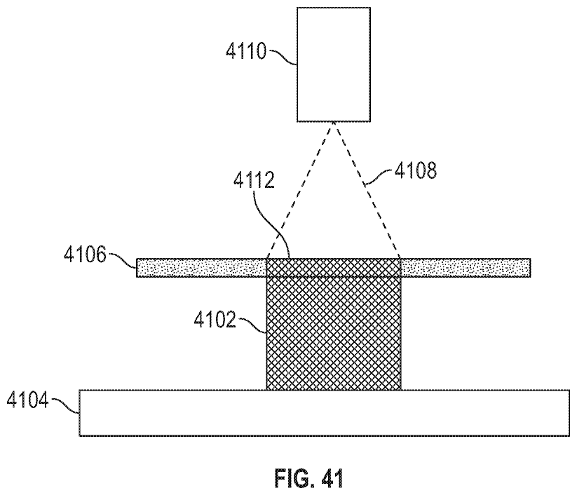

FIG. 41 is a partially schematic diagram providing a general overview of an additive manufacturing process, in accordance with embodiments of the present technology. Additive manufacturing (also referred to herein as “3D printing”) includes a variety of technologies which fabricate 3D objects directly from digital models through an additive process. For example, additive manufacturing can be used to directly fabricate orthodontic appliances (e.g., aligners, palatal expanders, retainers, attachment placement devices, attachments), restorative objects (e.g., crowns, veneers, implants), and/or other dental appliances (e.g., oral sleep apnea appliances, mouth guards). Additional examples of dental appliances and associated methods that are applicable to the present technology are described in Sections I-IV above.

In some embodiments, additive manufacturing includes depositing a precursor material (e.g., a polymeric resin) onto a build platform. The precursor material can be cured, polymerized, melted, sintered, fused, and/or otherwise solidified to form a portion of the object and/or to combine the portion with previously formed portions of the object. In some embodiments, the additive manufacturing techniques provided herein build up the object geometry in a layer-by-layer fashion, with successive layers being formed in discrete build steps. Alternatively or in combination, the additive manufacturing techniques described herein can allow for continuous build-up of an object geometry.

For example, in the embodiment of FIG. 41, an object 4102 is fabricated on a build platform 4104 from a series of cured material layers, with each layer having a geometry corresponding to a respective cross-section of the object 4102. To fabricate an individual object layer, a layer of curable material 4106 (e.g., polymerizable resin) is brought into contact with the build platform 4104 (when fabricating the first layer of the object 4102) or with the previously formed portion of the object 4102 on the build platform 4104 (when fabricating subsequent layers of the object 4102). In some embodiments, the curable material 4106 is formed on and supported by a substrate (not shown), such as a film. Energy 4108 (e.g., light) from an energy source 4110 (e.g., a laser, projector, or light engine) is then applied to the curable material 4106 to form a cured material layer 4112 on the build platform 4104 or on the object 4102. The remaining curable material 4106 can then be moved away from the build platform 4104 (e.g., by lowering the build platform 4104, by moving the build platform 4104 laterally, by raising the curable material 4106, and/or by moving the curable material 4106 laterally), thus leaving the cured material layer 4112 in place on the build platform 4104 and/or object 4102. The fabrication process can then be repeated with a fresh layer of curable material 4106 to build up the next layer of the object 4102.

The illustrated embodiment shows a “top down” configuration in which the energy source 4110 is positioned above and directs the energy 4108 down toward the build platform 4104, such that the object 4102 is formed on the upper surface of the build platform 4104. Accordingly, the build platform 4104 can be incrementally lowered relative to the energy source 4110 as successive layers of the object 4102 are formed. In other embodiments, however, the additive manufacturing process of FIG. 41 can be performed using a “bottom up” configuration in which the energy source 4110 is positioned below and directs the energy 4108 up toward the build platform 4104, such that the object 4102 is formed on the lower surface of the build platform 4104. Accordingly, the build platform 4104 can be incrementally raised relative to the energy source 4110 as successive layers of the object 4102 are formed.

Although FIG. 41 illustrates a representative example of an additive manufacturing process, this is not intended to be limiting, and the embodiments described herein can be adapted to other types of additive manufacturing systems (e.g., vat-based systems) and/or other types of additive manufacturing processes (e.g., material jetting, binder jetting, material extrusion, powder bed fusion, sheet lamination, directed energy deposition).

### Examples

The following examples are included to further describe some aspects of the present technology, and should not be used to limit the scope of the technology.

Example 1. A device for monitoring a subject's respiration, the device comprising:
a polymeric shell comprising a plurality of tooth-receiving cavities, wherein the polymeric shell is configured to be worn on the subject's teeth;
a force sensor coupled to the polymeric shell, wherein the force sensor is configured to measure force data associated with a deformation of the polymeric shell, and wherein the deformation corresponds to respiration of the subject;
one or more processors; and
a memory comprising instructions that, when executed by the one or more processors, cause the one or more processors to perform operations comprising:
   receiving the force data, and
   determining a respiratory parameter of the respiration based on the force data.

Example 2. The device of Example 1, wherein the respiratory parameter comprises one or more of the following: respiration rate, respiration rhythm, respiration depth, apnea events, hypopnea events, snoring, mouth breathing, or nasal breathing.

Example 3. The device of Example 1 or 2, wherein the deformation comprises one or more of thermal expansion or thermal contraction of the polymeric shell induced at least in part by the respiration.

Example 4. The device of any one of Examples 1 to 3, wherein determining the respiratory parameter comprises differentiating between mouth breathing and nasal breathing.

Example 5. The device of Example 4, wherein the differentiation is based on an amplitude of the force data.

Example 6. The device of Example 5, wherein the subject is determined to be mouth breathing if the amplitude exceeds a force threshold value, and wherein the subject is determined to be nasal breathing if the amplitude is below a force threshold value.

Example 7. The device of Example 6, wherein the force threshold value is determined based on historical respiratory data of the subject.

Example 8. The device of Example 7, wherein the historical respiratory data comprises first force data for a first period of time corresponding to mouth breathing and second force data for a second period of time corresponding to nasal breathing.

Example 9. The device of any one of Examples 1 to 8, wherein the polymeric shell further comprises a pocket, wherein the force sensor is disposed within the pocket, and wherein the force sensor is at least partially encapsulated by a thermosensitive material within the pocket.

Example 10. The device of Example 9, wherein the thermosensitive material is configured to expand or contract based at least in part on the respiration.

Example 11. The device of any one of Examples 1 to 10, further comprising a temperature sensor coupled to the polymeric shell, wherein the temperature sensor is configured to measure temperature data, and wherein the determining of the respiratory parameter is further based on the temperature data.

Example 12. The device of Example 11, wherein determining the respiratory parameter further comprises differentiating between mouth breathing and nasal breathing based on an average temperature of the temperature data, wherein the subject is determined to be nasal breathing if the average temperature exceeds a temperature threshold value, and wherein the subject is determined to be mouth breathing if the average temperature is below a temperature threshold value.

Example 13. The device of any one of Examples 1 to 12, further comprising a pressure sensor coupled to the polymeric shell, wherein the pressure sensor is configured to measure pressure data, and wherein the determining of the respiratory parameter is further based on the pressure data.

Example 14. The device of Example 13, wherein determining the respiratory parameter further comprises monitoring nasal breathing of the subject based on the pressure data.

Example 15. The device of Example 13 or 14, wherein the pressure sensor is encapsulated within a biocompatible material.

Example 16. The device of Example 15, wherein the biocompatible material is configured to inhibit fluids from entering the pressure sensor.

Example 17. The device of any one of Examples 1 to 16, wherein the operations further comprise outputting an indication of the determined respiratory parameter on a display.

Example 18. The device of any one of Examples 1 to 17, wherein the operations further comprise determining a disease or condition of the subject based on the determined respiratory parameter.

Example 19. The device of Example 18, wherein the disease or condition comprises one or more of asthma, pneumonia, hyperventilation, dyspnea, bradypnea, tachypnea, sleep apnea, insomnia, hypersomnia, parasomnia, narcolepsy, sleep paralysis, or snoring.

Example 20. The device of any one of Examples 1 to 19, wherein the operations further comprise outputting a treatment recommendation based on the determined respiratory parameter.

Example 21. The device of any one of Examples 1 to 20, wherein the operations further comprise detecting a bruxism event.

Example 22. The device of any one of Examples 1 to 21, wherein the operations further comprise detecting an activity level of the subject.

Example 23. The device of any one of Examples 1 to 22, wherein the one or more processors and the memory are coupled to the polymeric shell.

Example 24. The device of any one of Examples 1 to 22, wherein the one or more processors and the memory are part of an external device separate from the polymeric shell.

Example 25. The device of any one of Examples 1 to 24, wherein the polymeric shell is configured to retain the subject's teeth in a current arrangement.

Example 26. The device of any one of Examples 1 to 24, wherein the polymeric shell is configured to apply repositioning forces to one or more of the subject's teeth.

Example 27. The device of any one of Examples 1 to 26, wherein the polymeric shell is fabricated using an additive manufacturing process.

Example 28. The device of any one of Examples 1 to 26, wherein the polymeric shell is fabricated via thermoforming.

Example 29. The device of any one of Examples 1 to 28, wherein the device further comprises charging circuitry configured to be in electrical communication with an external charging device.

Example 30. The device of Example 29, wherein the charging circuitry is configured to wirelessly receive power from the external charging device.

Example 31. A system comprising the device of Example 29 or 30, and the external charging device.

Example 32. The system of Example 31, wherein the external charging device comprises a coupling mechanism that is configured to releasably couple to the polymeric shell.

Example 33. A computer-implemented method for monitoring a subject's respiration, the computer-implemented method comprising, by one or more processors:
receiving force data from a force sensor coupled to a polymeric shell worn on the subject's teeth, wherein the force data is associated with a deformation of the polymeric shell, and wherein the deformation corresponds to respiration of the subject; and
determining a respiratory parameter of the respiration based on the force data.

Example 34. The computer-implemented method of Example 33, wherein the respiratory parameter comprises one or more of the following: respiration rate, respiration rhythm, respiration depth, apnea events, hypopnea events, snoring, mouth breathing, or nasal breathing.

Example 35. The computer-implemented method of Example 33 or 34, wherein the deformation comprises one or more of thermal expansion or thermal contraction of the polymeric shell induced at least in part by the respiration.

Example 36. The computer-implemented method of any one of Examples 33 to 35, wherein determining the respiratory parameter comprises differentiating between mouth breathing and nasal breathing.

Example 37. The computer-implemented method of Example 36, wherein the differentiation is based on an amplitude of the force data.

Example 38. The computer-implemented method of Example 37, wherein the subject is determined to be mouth breathing if the amplitude exceeds a force threshold value, and wherein the subject is determined to be nasal breathing if the amplitude is below a force threshold value.

Example 39. The computer-implemented method of Example 38, wherein the force threshold value is determined based on historical respiratory data of the subject.

Example 40. The computer-implemented method of Example 39, wherein the historical respiratory data comprises first force data for a first period of time corresponding to mouth breathing and second force data for a second period of time corresponding to nasal breathing.

Example 41. The computer-implemented method of any one of Examples 33 to 40, wherein the polymeric shell further comprises a pocket, wherein the force sensor is disposed within the pocket, and wherein the force sensor is at least partially encapsulated by a thermosensitive material within the pocket.

Example 42. The computer-implemented method of Example 41, wherein the thermosensitive material is configured to expand or contract based at least in part on the respiration.

Example 43. The computer-implemented method of any one of Examples 33 to 42, further comprising:
receiving temperature data from a temperature sensor coupled to the polymeric shell, and
determining the respiratory parameter based on the temperature data.

Example 44. The computer-implemented method of Example 43, wherein the subject is determined to be nasal breathing if the temperature data includes a temperature that exceeds a temperature threshold value, and wherein the subject is determined to be mouth breathing if the temperature data includes a temperature that is below a temperature threshold value.

Example 45. The computer-implemented method of any one of Examples 33 to 44, further comprising:
receiving pressure data from a pressure sensor coupled to the polymeric shell, and
determining the respiratory parameter based on the pressure data.

Example 46. The computer-implemented method of Example 45, wherein the subject is determined to be mouth breathing if the pressure data includes a pressure that exceeds a threshold pressure value, and wherein the subject is determined to be nasal breathing if the pressure data includes a pressure that is below a threshold pressure value.

Example 47. The computer-implemented method of Example 45 or 46, wherein the pressure sensor is encapsulated within a biocompatible material.

Example 48. The computer-implemented method of Example 47, wherein the biocompatible material is configured to inhibit fluids from entering the pressure sensor.

Example 49. The computer-implemented method of any one of Examples 33 to 48, further comprising outputting an indication of the determined respiratory parameter on a display.

Example 50. The computer-implemented method of any one of Examples 33 to 49, further comprising determining a disease or condition of the subject based on the determined respiratory parameter.

Example 51. The computer-implemented method of Example 50, wherein the disease or condition comprises one or more of asthma, pneumonia, hyperventilation, dyspnea, bradypnea, tachypnea, sleep apnea, insomnia, hypersomnia, parasomnia, narcolepsy, sleep paralysis, or snoring.

Example 52. The computer-implemented method of any one of Examples 33 to 51, further comprising outputting a treatment recommendation based on the determined respiratory parameter.

Example 53. The computer-implemented method of any one of Examples 33 to 52, further comprising detecting a bruxism event.

Example 54. The computer-implemented method of any one of Examples 33 to 53, further comprising detecting an activity level of the subject.

Example 55. The computer-implemented method of any one of Examples 33 to 54, wherein the polymeric shell is configured to retain the subject's teeth in a current arrangement.

Example 56. The computer-implemented method of any one of Examples 33 to 54, wherein the polymeric shell is configured to apply repositioning forces to one or more of the subject's teeth.

Example 57. A device for monitoring a subject's cardiac activity, the device comprising:
a polymeric shell comprising a plurality of tooth-receiving cavities, wherein the polymeric shell is configured to be worn on the subject's teeth;
a motion sensor coupled to the polymeric shell, the motion sensor configured to measure motion data associated with blood flow in the subject's head;
one or more processors; and
a memory comprising instructions that, when executed by the one or more processors, cause the one or more processors to perform operations comprising:
   receiving the motion data, and
   determining a cardiac parameter based on the motion data.

Example 58. The device of Example 57, wherein the motion data comprises changes in head position caused by the blood flow in the subject's head.

Example 59. The device of Example 58, wherein the blood flow comprises blood flow in a carotid artery of the subject.

Example 60. The device of Example 58 or 59, wherein the motion data comprises head ballistocardiography (BCG) data.

Example 61. The device of any one of Examples 57 to 60, wherein the motion sensor comprises one or more of an inertial measurement unit, a piezoelectric sensor, or an electromechanical film.

Example 62. The device of any one of Examples 57 to 61, wherein the polymeric shell further comprises a pocket, wherein the motion sensor is disposed within the pocket, and wherein the motion sensor is at least partially encapsulated by a biocompatible material within the pocket.

Example 63. The device of any one of Examples 57 to 62, further comprising at least one additional sensor coupled to the polymeric shell, wherein the at least one sensor is configured to generate additional sensor data, and wherein the determining of the cardiac parameter is further based on the additional sensor data.

Example 64. The device of Example 63, wherein the at least one additional sensor comprises one or more of a temperature sensor, force sensor, or pressure sensor.

Example 65. The device of any one of Examples 57 to 64, wherein the cardiac parameter comprises one or more of flow time, stroke volume, stroke volume variation, stroke volume index, cardiac output, heart rate, heart rate variability, systolic pressure, diastolic pressure, mean arterial pressure, or pulse pressure.

Example 66. The device of Example 65, wherein the cardiac parameter comprises heart rate, and wherein the heart rate is determined based on a vertical component of the motion data.

Example 67. The device of any one of Examples 57 to 66, wherein the operations further comprise outputting an indication of the determined cardiac parameter on a display.

Example 68. The device of any one of Examples 57 to 67, wherein the operations further comprise determining a disease or condition of the subject based on the cardiac parameter.

Example 69. The device of Example 68, wherein the disease or condition comprises one or more of high blood pressure, low blood pressure, arrhythmia, atrial fibrillation, tachycardia, heart failure, heart attack, stroke, congenital heart defect, or coronary artery disease.

Example 70. The device of any one of Examples 57 to 69, further comprising outputting a treatment recommendation based on the cardiac parameter.

Example 71. The device of any one of Examples 57 to 70, wherein the operations further comprise determining a respiratory parameter of the subject's respiration based on the motion data.

Example 72. The device of Example 71, wherein the respiratory parameter is determined based on a low frequency oscillatory component of the motion data.

Example 73. The device of any one of Examples 57 to 72, wherein the operations further comprise detecting a bruxism event based on the motion data.

Example 74. The device of any one of Examples 57 to 73, wherein the operations further comprise diagnosing a sleep disorder or condition based on the motion data.

Example 75. The device of Example 74, wherein the sleep disorder or condition comprises one or more of sleep apnea, insomnia, hypersomnia, parasomnia, narcolepsy, sleep paralysis, or snoring.

Example 76. The device of any one of Examples 57 to 75, wherein the operations further comprise detecting an activity level of the subject.

Example 77. The device of any one of Examples 57 to 76, wherein the one or more processors and the memory are coupled to the polymeric shell.

Example 78. The device of any one of Examples 57 to 76, wherein the one or more processors and the memory are part of an external device separate from the polymeric shell.

Example 79. The device of any one of Examples 57 to 78, wherein the polymeric shell is configured to retain the subject's teeth in a current arrangement.

Example 80. The device of any one of Examples 57 to 78, wherein the polymeric shell is configured to apply repositioning forces to one or more of the subject's teeth.

Example 81. The device of any one of Examples 57 to 80, wherein the polymeric shell is fabricated using an additive manufacturing process.

Example 82. The device of any one of Example 57 to 80, wherein the polymeric shell is fabricated via thermoforming.

Example 83. The device of any one of Examples 57 to 82, wherein the device further comprises charging circuitry configured to be in electrical communication with an external charging device.

Example 84. The device of Example 83, wherein the charging circuitry is configured to wirelessly receive power from the external charging device.

Example 85. A system comprising the device of Example 83 or 84, and the external charging device.

Example 86. The system of Example 85, wherein the external charging device comprises a coupling mechanism that is configured to releasably couple to the polymeric shell.

Example 87. A computer-implemented method for monitoring a subject's cardiac activity, the computer-implemented method comprising, by one or more processors:
receiving motion data from a motion sensor coupled to a polymeric shell worn on the subject's teeth, wherein the motion data is associated with blood flow in the subject's head; and
determining a cardiac parameter based on the motion data.

Example 88. The computer-implemented method of Example 87, wherein the motion data comprises changes in head position caused by the blood flow in the subject's head.

Example 89. The computer-implemented method of Example 88, wherein the blood flow comprises blood flow in a carotid artery of the subject.

Example 90. The computer-implemented method of Example 88 or 89, wherein the motion data comprises head ballistocardiography (BCG) data.

Example 91. The computer-implemented method of Example 87, wherein the motion sensor comprises one or more of an inertial measurement unit, a piezoelectric sensor, or an electromechanical film.

Example 92. The computer-implemented method of any one of Examples 87 to 91, wherein the polymeric shell further comprises a pocket, wherein the motion sensor is disposed within the pocket, and wherein the motion sensor is at least partially encapsulated by a biocompatible material within the pocket.

Example 93. The computer-implemented method of any one of Examples 87 to 92, further comprising:
receiving additional sensor data from at least one additional sensor coupled to the polymeric shell, and
determining the cardiac parameter based on the additional sensor data.

Example 94. The computer-implemented method of Example 93, wherein the at least one additional sensor comprises one or more of a temperature sensor, force sensor, or pressure sensor.

Example 95. The computer-implemented method of any one of Examples 87 to 94, wherein the cardiac parameter comprises one or more of flow time, stroke volume, stroke volume variation, stroke volume index, cardiac output, heart rate, heart rate variability, systolic pressure, diastolic pressure, mean arterial pressure, or pulse pressure.

Example 96. The computer-implemented method of Example 95, wherein the cardiac parameter comprises heart rate, and wherein the heart rate is determined based on motion data comprising blood flow in a substantially vertical direction.

Example 97. The computer-implemented method of any one of Examples 87 to 96, further comprising outputting an indication of the determined cardiac parameter on a display.

Example 98. The computer-implemented method of any one of Examples 87 to 97, further comprising determining a disease or condition of the subject based on the cardiac parameter.

Example 99. The computer-implemented method of Example 98, wherein the disease or condition comprises one or more of high blood pressure, low blood pressure, arrhythmia, atrial fibrillation, tachycardia, heart failure, heart attack, stroke, congenital heart defect, or coronary artery disease.

Example 100. The computer-implemented method of any one of Examples 87 to 99, further comprising outputting a treatment recommendation based on the cardiac parameter.

Example 101. The computer-implemented method of any one of Examples 87 to 100, further comprising determining a respiratory parameter of the subject's respiration based on the motion data.

Example 102. The computer-implemented method of Example 101, wherein the respiratory parameter is determined based on a low frequency oscillatory component of the motion data.

Example 103. The computer-implemented method of any one of Examples 87 to 102, further comprising detecting a bruxism event based on the motion data.

Example 104. The computer-implemented method of any one of Examples 87 to 103, further comprising diagnosing a sleep disorder or condition based on the motion data.

Example 105. The computer-implemented method of Example 104, wherein the sleep disorder or condition comprises one or more of sleep apnea, insomnia, hypersomnia, parasomnia, narcolepsy, sleep paralysis, or snoring.

Example 106. The computer-implemented method of any one of Examples 87 to 105, further comprising detecting a bruxism event.

Example 107. The computer-implemented method of any one of Examples 87 to 106, further comprising detecting an activity level of the subject.

Example 108. The computer-implemented method of any one of Examples 87 to 107, wherein the polymeric shell is configured to retain the subject's teeth in a current arrangement.

Example 109. The computer-implemented method of any one of Examples 87 to 107, wherein the polymeric shell is configured to apply repositioning forces to one or more of the subject's teeth.

Example 110. A device for monitoring physiological parameters of a subject from within an intraoral cavity of the subject, wherein the device is made by a process comprising:
providing a polymeric shell comprising a plurality of tooth-receiving cavities and a pocket;
disposing a sensor assembly into the pocket, wherein the sensor assembly comprises one or more of a force sensor, a pressure sensor, a motion sensor, or a temperature sensor; and
sealing the pocket, wherein the sealing comprises disposing a biocompatible material into the pocket to at least partially encapsulate the sensor assembly.

Example 111. The device of Example 110, wherein the pocket is disposed along a lingual side of the polymeric shell.

Example 112. The device of Example 110 or 111, wherein the pocket is on one of a left side or a right side of the polymeric shell.

Example 113. The device of any one of Examples 110 to 112, wherein the pocket is disposed along a buccal side of the polymeric shell.

Example 114. The device of any one of Examples 110 to 113, wherein the sensor assembly comprises the pressure sensor, and wherein sealing the pocket further comprises building in an air pocket around at least a portion of the pressure sensor.

Example 115. The device of any one of Examples 110 to 114, wherein the sensor assembly is coupled to charging circuitry within the pocket, the charging circuitry configured to be in electrical communication with an external charging device.

Example 116. The device of Example 115, wherein the charging circuitry is configured to wirelessly receive power from the external charging device.

Example 117. A system comprising the device of Example 115 or 116, and the external charging device.

Example 118. The system of Example 117, wherein the external charging device is part of a case for the device.

Example 119. The system of Example 117 or 118, wherein the external charging device comprises a coupling mechanism that is configured to releasably couple to the device.

Example 120. The system of Example 119, wherein the charging circuitry comprises a first coil and the external charging device comprises a second coil, and wherein when the device is coupled to the coupling mechanism, the first coil is aligned with the second coil.

Example 121. A device for monitoring a subject, the device comprising:
a polymeric shell comprising a plurality of tooth-receiving cavities, wherein the polymeric shell is configured to be worn on the subject's teeth;
at least one sensor coupled to the polymeric shell;
one or more processors; and
a memory comprising instructions that, when executed by the one or more processors, cause the one or more processors to perform operations comprising:
   receiving sensor data from the at least one sensor, and
   determining a physiological parameter of the subject based on the sensor data.

Example 122. The device of Example 121, wherein the at least one sensor comprises one or more of the following: a force sensor, a pressure sensor, a temperature sensor, a motion sensor, a humidity sensor, a proximity sensor, an optical sensor, or a contact sensor.

Example 123. The device of Example 121 or 122, wherein the at least one sensor is configured to monitor at least one orofacial activity of the subject.

Example 124. The device of Example 123, wherein the at least one orofacial activity comprises one or more of the following: swallowing and/or saliva-related behaviors, tongue behaviors, jaw behaviors, lip and/or cheek-related behaviors, coughing, throat clearing, yawning, snoring, speaking, breathing, breath holding, or gasping.

Example 125. The device of Example 123 or 124, wherein the physiological parameter includes the at least one orofacial activity or is determined based on the at least one orofacial activity.

Example 126. The device of any one of Examples 123 to 125, wherein the operations further comprise:
determining a timing of the at least one orofacial activity, and
identifying a root cause of the at least one orofacial activity based on the determined timing.

Example 127. The device of Example 126, wherein the root cause is identified based on correlations in timing of a plurality of orofacial activities.

Example 128. The device of Example 126 or 127, wherein the operations further comprise outputting an indication of the determined timing on a display.

Example 129. The device of any one of Examples 121 to 128, wherein the operations further comprise determining a disease or condition of the subject based on the determined physiological parameters.

Example 130. The device of any one of Examples 121 to 129, wherein the operations further comprise outputting a treatment recommendation based on the determined physiological parameter.

Example 131. A computer-implemented method for monitoring a subject, the computer-implemented method comprising, by one or more processors:
receiving sensor data from at least one sensor coupled to a polymeric shell worn on the subject's teeth; and
determining a physiological parameter of the subject based on the sensor data.

Example 132. The computer-implemented method of Example 131, wherein the at least one sensor comprises one or more of the following: a force sensor, a pressure sensor, a temperature sensor, a motion sensor, a humidity sensor, a proximity sensor, an optical sensor, or a contact sensor.

Example 133. The computer-implemented method of Example 131 or 132, wherein the at least one sensor is configured to monitor at least one orofacial activity of the subject.

Example 134. The computer-implemented method of Example 133, wherein the at least one orofacial activity comprises one or more of the following: swallowing and/or saliva-related behaviors, tongue behaviors, jaw behaviors, lip and/or cheek-related behaviors, coughing, throat clearing, yawning, snoring, speaking, breathing, breath holding, or gasping.

Example 135. The computer-implemented method of Example 133 or 134, wherein the physiological parameter includes the at least one orofacial activity or is determined based on the at least one orofacial activity.

Example 136. The computer-implemented method of any one of Examples 133 to 135, further comprising:
determining a timing of the at least one orofacial activity, and
identifying a root cause of the at least one orofacial activity based on the determined timing.

Example 137. The computer-implemented method of Example 136, wherein the root cause is identified based on correlations in timing of a plurality of orofacial activities.

Example 138. The computer-implemented method of Example 136 or 137, further comprising outputting an indication of the determined timing on a display.

Example 139. The computer-implemented method of any one of Examples 131 to 138, further comprising determining a disease or condition of the subject based on the determined physiological parameters.

Example 140. The computer-implemented method of any one of Examples 131 to 139, further comprising outputting a treatment recommendation based on the determined physiological parameter.

### CONCLUSION

Although many of the embodiments are described above with respect to systems, devices, and methods for intraoral monitoring, the technology is applicable to other applications and/or other approaches, such as monitoring at other anatomical locations. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-41.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A device for monitoring a subject's respiration, the device comprising:
a polymeric shell comprising a plurality of tooth-receiving cavities, wherein the polymeric shell is configured to be worn on the subject's teeth;
a force sensor coupled to the polymeric shell, wherein the force sensor is configured to measure force data associated with a deformation of the polymeric shell, and wherein the deformation corresponds to respiration of the subject;
one or more processors; and
a memory comprising instructions that, when executed by the one or more processors, cause the one or more processors to perform operations comprising:
receiving the force data, and
determining a respiratory parameter of the respiration based on the force data.

2. The device of claim 1, wherein the respiratory parameter comprises one or more of the following: respiration rate, respiration rhythm, respiration depth, apnea events, hypopnea events, snoring, mouth breathing, or nasal breathing.

3. The device of claim 1, wherein the deformation comprises one or more of thermal expansion or thermal contraction of the polymeric shell induced at least in part by the respiration.

4. The device of claim 1, wherein determining the respiratory parameter comprises differentiating between mouth breathing and nasal breathing.

5. The device of claim 4, wherein the differentiation is based on an amplitude of the force data.

6. The device of claim 5, wherein the subject is determined to be mouth breathing if the amplitude exceeds a force threshold value, and wherein the subject is determined to be nasal breathing if the amplitude is below a force threshold value.

7. The device of claim 1, wherein the polymeric shell further comprises a pocket, wherein the force sensor is disposed within the pocket, and wherein the force sensor is at least partially encapsulated by a thermosensitive material within the pocket.

8. The device of claim 7, wherein the thermosensitive material is configured to expand or contract based at least in part on the respiration.

9. The device of claim 1, wherein the operations further comprise outputting an indication of the determined respiratory parameter on a display.

10. The device of claim 1, wherein the operations further comprise determining a disease or condition of the subject based on the determined respiratory parameter.

11. A computer-implemented method for monitoring a subject's respiration, the computer-implemented method comprising, by one or more processors:
receiving force data from a force sensor coupled to a polymeric shell worn on the subject's teeth, wherein the force data is associated with a deformation of the polymeric shell, and wherein the deformation corresponds to respiration of the subject; and
determining a respiratory parameter of the respiration based on the force data.

12. The computer-implemented method of claim 11, wherein the respiratory parameter comprises one or more of the following: respiration rate, respiration rhythm, respiration depth, apnea events, hypopnea events, snoring, mouth breathing, or nasal breathing.

13. The computer-implemented method of claim 11, wherein the deformation comprises one or more of thermal expansion or thermal contraction of the polymeric shell induced at least in part by the respiration.

14. The computer-implemented method of claim 11, wherein determining the respiratory parameter comprises differentiating between mouth breathing and nasal breathing.

15. The computer-implemented method of claim 14, wherein the differentiation is based on an amplitude of the force data.

16. The computer-implemented method of claim 15, wherein the subject is determined to be mouth breathing if the amplitude exceeds a force threshold value, and wherein the subject is determined to be nasal breathing if the amplitude is below a force threshold value.

17. The computer-implemented method of claim 11, further comprising outputting an indication of the determined respiratory parameter on a display.

18. The computer-implemented method of claim 11, further comprising determining a disease or condition of the subject based on the determined respiratory parameter.

19. The computer-implemented method of claim 18, wherein the disease or condition comprises one or more of asthma, pneumonia, hyperventilation, dyspnea, bradypnea, tachypnea, sleep apnea, insomnia, hypersomnia, parasomnia, narcolepsy, sleep paralysis, or snoring.

20. The computer-implemented method of claim 11, further comprising outputting a treatment recommendation based on the determined respiratory parameter.
